SEQUENCE LISTING (125 sequences)

SEQ ID NO: 1 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 2 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 3 - protein, 5 aa, synthetic construct
SYAMS

SEQ ID NO: 4 - protein, 17 aa, synthetic construct
AIIGSGASTYYADSVKG

SEQ ID NO: 5 - protein, 8 aa, synthetic construct
GWFGGFNY

SEQ ID NO: 6 - protein, 12 aa, synthetic construct
RASQSVTSSYLA

SEQ ID NO: 7 - protein, 7 aa, synthetic construct
VGSRRAT

SEQ ID NO: 8 - protein, 9 aa, synthetic construct
QQGIMLPPT

SEQ ID NO: 9 - protein, 117 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 10 - protein, 108 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 11 - protein, 5 aa, synthetic construct
SHAMS

SEQ ID NO: 12 - protein, 16 aa, synthetic construct
AIWASGEQYYADSVKG

SEQ ID NO: 13 - protein, 8 aa, synthetic construct
GWLGNFDY

SEQ ID NO: 14 - protein, 12 aa, synthetic construct
RASQSVSRSYLA

SEQ ID NO: 15 - protein, 7 aa, synthetic construct
GASTRAT

SEQ ID NO: 16 - protein, 9 aa, synthetic construct
QQGQVIPPT

SEQ ID NO: 17 - protein, 116 aa, synthetic construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 18 - protein, 108 aa, synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 19 - protein, 5 aa, synthetic construct
SYTMN

SEQ ID NO: 20 - protein, 17 aa, synthetic construct
GVAPSGDRTYYTDSVKG

SEQ ID NO: 21 - protein, 6 aa, synthetic construct
GFAFTN

SEQ ID NO: 22 - protein, 11 aa, synthetic construct
RASQGISSALA

SEQ ID NO: 23 - protein, 7 aa, synthetic construct
DASSLDS

SEQ ID NO: 24 - protein, 9 aa, synthetic construct
QQYNNYPLT

SEQ ID NO: 25 - protein, 115 aa, synthetic construct
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSS

SEQ ID NO: 26 - protein, 107 aa, synthetic construct
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMK

SEQ ID NO: 27 - protein, 5 aa, synthetic construct
TYAMN

SEQ ID NO: 28 - protein, 19 aa, synthetic construct
RIRSKYNNYATYYADSVKG

SEQ ID NO: 29 - protein, 14 aa, synthetic construct
HGNFGNSYVSWFAY

SEQ ID NO: 30 - protein, 14 aa, synthetic construct
GSSTGAVTTSNYAN

SEQ ID NO: 31 - protein, 7 aa, synthetic construct
GTNKRAP

SEQ ID NO: 32 - protein, 9 aa, synthetic construct
ALWYSNLWV

SEQ ID NO: 33 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 34 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 35 - protein, 5 aa, synthetic construct
GYGMH

SEQ ID NO: 36 - protein, 17 aa, synthetic construct
YITSSSINIKYADAVKG

SEQ ID NO: 37 - protein, 7 aa, synthetic construct
FDWDKNY

SEQ ID NO: 38 - protein, 11 aa, synthetic construct
QASQDISNYLN

SEQ ID NO: 39 - protein, 7 aa, synthetic construct
YTNKLAD

SEQ ID NO: 40 - protein, 9 aa, synthetic construct
QQYYNYPWT

SEQ ID NO: 41 - protein, 116 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 42 - protein, 107 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 43 - protein, 5 aa, synthetic construct
EFGMN

SEQ ID NO: 44 - protein, 17 aa, synthetic construct
WINTKTGEATYVEEFKG

SEQ ID NO: 45 - protein, 12 aa, synthetic construct
WDFAYYVEAMDY

SEQ ID NO: 46 - protein, 11 aa, synthetic construct
KASAAVGTYVA

SEQ ID NO: 47 - protein, 7 aa, synthetic construct
SASYRKR

SEQ ID NO: 48 - protein, 10 aa, synthetic construct
HQYYTYPLFT

SEQ ID NO: 49 - protein, 121 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 50 - protein, 108 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 51 - protein, 678 aa, synthetic construct
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 52 - protein, 222 aa, synthetic construct
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 438 aa, synthetic construct
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 54 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 681 aa, synthetic construct
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - protein, 214 aa, synthetic construct
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKRADAAPTVSIFPPSSRKLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 57 - protein, 438 aa, synthetic construct
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 58 - protein, 212 aa, synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 59 - protein, 215 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 214 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 693 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 450 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 63 - protein, 215 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 64 - protein, 211 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 65 - protein, 677 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSQVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 66 - protein, 444 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 67 - protein, 419 aa, synthetic construct
QTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKASGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 68 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 69 - DNA, 37 bp, synthetic construct
  source 1..37: mol_type = other DNA
aagcttgccaccatggagactgggctgcgctggcttc

SEQ ID NO: 70 - DNA, 40 bp, synthetic construct
  source 1..40: mol_type = other DNA
cttgggcccttggtacttgctgaggagacggtgaccaggg

SEQ ID NO: 71 - DNA, 40 bp, synthetic construct
  source 1..40: mol_type = other DNA
cttgggcccttggtacttgctgaggagacggtgaccgtgg

SEQ ID NO: 72 - DNA, 42 bp, synthetic construct
  source 1..42: mol_type = other DNA
acagaggaagcttgccgccaccatggacatgagggtccccgc

SEQ ID NO: 73 - DNA, 24 bp, synthetic construct
  source 1..24: mol_type = other DNA
tgaagacagatggtgcagccacag

SEQ ID NO: 74 - protein, 768 aa, synthetic construct
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSVDEQLYFQGGSGLNDIFEAQKIEWHEARAHHHHHH

SEQ ID NO: 75 - protein, 761 aa, synthetic construct
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 76 - protein, 770 aa, synthetic construct
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDVDEQLYFQGGSGLNDIFEAQKIEWHEARAHHHHHH

SEQ ID NO: 77 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 78 - protein, 201 aa, Homo sapiens
MQSGTHWRVLGLCLLSVGVWGQDGNEEMAYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 79 - protein, 198 aa, synthetic construct
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 80 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 81 - protein, 5 aa, synthetic construct
EPKSC

SEQ ID NO: 82 - protein, 24 aa, synthetic construct
DKTHTCPXCPWITHXBEINGSORP

SEQ ID NO: 83 - protein, 107 aa, synthetic construct
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 84 - protein, 105 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 85 - protein, 330 aa, Homo sapiens [sequence omitted]

SEQ ID NO: 86 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 88 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 89 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 253 aa, Homo sapiens
MWQLLLPTALLLLVSAGMRTDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 91 - protein, 415 aa, Mus musculus
MRLPRASSPCGLAWGPLLLGLSGLLVASQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWMRHPSLCRKLGTLLKRHPEGEESPPCPAPRADPHFPDLAEPLLPMSGDLSPSPAGPPTAPSLEEVVLQQQSPLVQARELEAEPGEHGQVAHGANGIHVTGGSVTVTGNIYIYNGPVLGGTRGPGDPPAPPEPPYPTPEEGAPGPSELSTPYQEDGKAWHLAETETLGCQDL

SEQ ID NO: 92 - protein, 5 aa, synthetic construct
GGGGS

SEQ ID NO: 93 - protein, 10 aa, synthetic construct
GGGGSGGGGS

SEQ ID NO: 94 - protein, 10 aa, synthetic construct
SGGGGSGGGG

SEQ ID NO: 95 - protein, 14 aa, synthetic construct
GGGGSGGGGSGGGG

SEQ ID NO: 96 - protein, 10 aa, synthetic construct
GSPGSSSSGS

SEQ ID NO: 97 - protein, 15 aa, synthetic construct
GGGGSGGGGSGGGGS

SEQ ID NO: 98 - protein, 20 aa, synthetic construct
GGGGSGGGGSGGGGSGGGGS

SEQ ID NO: 99 - protein, 8 aa, synthetic construct
GSGSGSGS

SEQ ID NO: 100 - protein, 8 aa, synthetic construct
GSGSGNGS

SEQ ID NO: 101 - protein, 8 aa, synthetic construct
GGSGSGSG

SEQ ID NO: 102 - protein, 6 aa, synthetic construct
GGSGSG

SEQ ID NO: 103 - protein, 4 aa, synthetic construct
GGSG

SEQ ID NO: 104 - protein, 8 aa, synthetic construct
GGSGNGSG

SEQ ID NO: 105 - protein, 8 aa, synthetic construct
GGNGSGSG

SEQ ID NO: 106 - protein, 6 aa, synthetic construct
GGNGSG

SEQ ID NO: 107 - protein, 167 aa, Homo sapiens
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 108 - protein, 5 aa, synthetic construct
SYAMN

SEQ ID NO: 109 - protein, 19 aa, synthetic construct
RIRSKYNNYATYYADSVKG

SEQ ID NO: 110 - protein, 14 aa, synthetic construct
ASNFPASYVSYFAY

SEQ ID NO: 111 - protein, 5 aa, synthetic construct
NAWMS

SEQ ID NO: 112 - protein, 19 aa, synthetic construct
RIKSKTDGGTTDYAAPVKG

SEQ ID NO: 113 - protein, 9 aa, synthetic construct
PWEWSWYDY

SEQ ID NO: 114 - protein, 14 aa, synthetic construct
GSSTGAVTTSNYAN

SEQ ID NO: 115 - protein, 7 aa, synthetic construct
GTNKRAP

SEQ ID NO: 116 - protein, 9 aa, synthetic construct
ALWYSNLWV

SEQ ID NO: 117 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 118 - protein, 120 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSS

SEQ ID NO: 119 - protein, 109 aa, synthetic construct; identical to SEQ ID NO: 34

SEQ ID NO: 120 - protein, 688 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 449 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 122 - protein, 215 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 123 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 124 - protein, 255 aa, Mus musculus
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 125 - protein, 257 aa, Macaca fascicularis
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

(19) United States
(12) Patent Application Publication
(10) Pub. No.: US 2026/0109767 A1
(43) Pub. Date: Apr. 23, 2026

(54) COMBINATION THERAPY WITH FAP-TARGETED LYMPHOTOXIN BETA RECEPTOR AGONISTS

(71) Applicant: Hoffmann-La Roche Inc., Little Falls, NJ (US)

(72) Inventors: Roberta BIANCHI, Zürich (CH); Ralf Joerg HOSSE, Mettmenstetten (CH); Leo Frederik KUNZ, Basel (CH); Pablo UMAÑA FERNÁNDEZ, Lachen (CH)

(73) Assignee: Hoffmann-La Roche Inc., Little Falls, NJ (US)

(21) Appl. No.: 19/424,027

(22) Filed: Dec. 17, 2025

Related U.S. Application Data
(63) Continuation of application No. PCT/EP2024/067018, filed on Jun. 19, 2024.

(30) Foreign Application Priority Data
Jun. 21, 2023 (EP) 23180654.8

(51) Int. Cl. C07K 16/28; A61K 39/00; A61P 35/00; C07K 16/30

(52) U.S. Cl. CPC C07K 16/2809; A61P 35/00; C07K 16/2878; C07K 16/3007; A61K 2039/507; C07K 2317/31; C07K 2317/52; C07K 2317/55; C07K 2317/565; C07K 2317/75

(57) ABSTRACT

The present invention relates to combination therapies employing tumor targeted anti-CD3 bispecific antibodies (T cell engager) and FAP-targeted LTBR antibodies, the use of these combination therapies for the treatment of cancer and methods of using the combination therapies.

### CROSS REFERENCE TO RELATED APPLICATIONS

This application is a continuation of International Patent Application No. PCT/EP2024/067018, filed Jun. 19, 2024, claiming priority to European Patent Application No. 23180654.8 filed Jun. 21, 2023, which are incorporated herein by reference in its entirety.

### SEQUENCE LISTING

This application contains a Sequence Listing which has been submitted electronically in XML format and is hereby incorporated by reference in its entirety. Said XML copy, created on Dec. 11, 2025, is named P38606-US-1 Sequence Listing.xml and is 127,065 bytes in size.

### FIELD OF THE INVENTION

The present invention relates to combination therapies employing tumor targeted anti-CD3 bispecific antibodies (T cell engager) and FAP-targeted LTBR antibodies, the use of these combination therapies for the treatment of cancer and methods of using the combination therapies.

### BACKGROUND

Despite advances in treatment options, such as the approval of cancer immunotherapies (i.e. checkpoint inhibitors, T-cell engagers and cell therapies) cancer remains one of the leading causes of death worldwide. Therefore, there is a high medical need for optimal therapy combinations to increase survival of cancer patients.

Cancer immunotherapies comprise checkpoint inhibitors (CPIs) designed to remove inhibitory signals of T-cell activation, unleashing endogenous tumor-reactive T cells to mount an effective antitumor response. These cancer immunotherapies can achieve durable responses in some cancer indications and are therefore emerging as a new standard of care. However, only a subset of patients (<30%) experience a durable benefit from these therapies and the majority of patients relapse due to primary or acquired resistance mechanisms. In addition, several common cancer indications (e.g. colorectal cancer and pancreatic cancer) are mostly refractory to these immunomodulators due to their poor immunogenicity and poorly infiltrated immune phenotype. Consequently, there is an urgent medical need in developing therapies to tackle resistance mechanisms and increase responses to checkpoint inhibitors.

An alternative approach to activate a patient's T cells to attack tumor cells is the use of bispecific T cell engagers. T cell engagers work by redirecting T cells to cancer cells by binding to CD3 on T cells and a tumor associated antigen on tumor cells. This T cell engagement leads to T cell activation resulting in tumor cell killing, which is independent of antigen presentation on the major histocompatibility complex. Bispecific T cell engagers have shown clinical activity in hematological malignancies, but successful clinical development in solid tumors has been so far challenging. Many of the mechanisms that limit the activity of other immunotherapies in solid tumors, such as the immunosuppressive tumor microenvironment and poor T cell availability, may also impact the activity of bispecific T cell-engagers.

Clinical data shows that patients who do not respond or respond poorly to CPIs display a non T-cell inflamed immune phenotype characterized either by the absence of cytotoxic T cells, or by their restricted localization to the tumor stroma (Galon and Bruni, Nat Rev Drug Discov. 2019, 18(3), 197-218; Hedge et al, Clin Cancer Res. 2016, 22(8), 1865-1874). Moreover, it has been shown preclinically that tumors with a high density of T cell infiltrate at baseline had a stronger response to T cell engagers as compared to those with a low density T cell infiltrate at baseline (Belmontes et al, Sci Transl Med. 2021, 13(608):eabd1524).

Therefore, there is a need for novel therapies that increase immune infiltration. These therapies would be of great utility in improving the response rate and expanding the clinical benefit to T cell engagers.

Activation of the lymphotoxin beta receptor (LTBR) is emerging as a strategy to improve immune infiltration by inducing the upregulation of adhesion molecules and chemoattractants and the differentiation of vessels to high endothelial venules (HEVs). HEVs are specialized vessels that facilitate the transmigration of lymphocytes in secondary lymphoid organs. HEVs are also found in human and murine tumors and their presence correlates with an increased immune infiltrate.

LTBR activation by means of an agonistic antibody or a targeted ligand, increased T cell infiltration in several mouse tumor models (Lukashev et al, Cancer Res. 2006, 66(19), 9617-9624; Allen et al, Sci Transl Med. 2017, 9(385), eaak9679; Johansson-Percival et al, Nat Immunol. 2017, 18(11), 1207-1217; Tang et al, Cancer Cell 2016, 29(3), 285-296).

LTBR is a widely expressed target. A phase I study assessed the safety and tolerability of an LTBR agonistic humanized antibody (hCBE11) in patients with advanced solid tumors (ClinicalTrials.gov, NCT00105170). The study was suspended and subsequently terminated before completing the patient enrollment. This suggests the potential for serious safety issues associated with widespread LTBR agonism in humans. Therefore it would be advantageous, to induce LTBR agonism exclusively at the tumor site. Local drug delivery can be achieved by intratumoral injection, however this route of administration can pose significant logistical challenges for the hospital and has a higher burden for the patient, especially when frequent administrations are needed and when the lesions are deep, visceral and only accessible upon imaging guidance and patient sedation. Due to this limitation, systemic administration of an LTBR agonist that only acts at the tumor site would be desired.

### SUMMARY OF THE INVENTION

We herein describe a novel combination therapy employing a tumor targeted LTBR agonist in combination with a T cell engager and show that the combination therapy is superior to either monotherapy in controlling tumor growth and improving survival.

The present invention relates to T-cell engaging anti-CD3 bispecific antibodies and their use in combination with FAP-targeted LTBR agonistic antibody, in particular to their use in a method for treating or delaying progression of cancer, more particularly for the treatment of advanced and/or metastatic solid tumors. It has been found that the combination therapy described herein is more effective in inhibiting tumor growth, eliminating tumor cells and improving survival than treatment with the T-cell engaging anti-CD3 bispecific antibodies (in particular anti-CEA/anti-CD3 bispecific antibodies or anti-FolR1/anti-CD3 bispecific antibodies) alone. Moreover, only the combination therapy surprisingly induces a more inflamed, less immunosuppressive and highly HEV (high endothelial venules) enriched tumor microenvironment.

In one aspect, the invention provides a T-cell engaging anti-CD3 bispecific antibody in combination with a FAP-targeted lymphotoxin beta receptor (LTBR) agonistic antibody for use in a combination therapy for the treatment of cancer in an individual. In one aspect, the T-cell engaging anti-CD3 bispecific antibody and the FAP-targeted LTBR agonistic antibody are administered together in a single composition or administered separately in two or more different compositions. Particularly, the T-cell engaging anti-CD3 bispecific antibody binds specifically to a tumor-associated antigen, for instance Carcinoembryonic Antigen (CEA). In a further aspect, the T-cell engaging anti-CD3 bispecific antibody is an anti-CEA/anti-CD3 bispecific antibody. Thus, the invention provides an anti-CEA/anti-CD3 bispecific antibody for use in a combination therapy for the treatment of cancer in an individual, wherein the anti-CEA/anti-CD3 bispecific antibody is used in combination with a FAP-targeted LTBR agonistic antibody. In another aspect, the T-cell engaging anti-CD3 bispecific antibody is an anti-FolR1/anti-CD3 bispecific antibody. Thus, the invention provides an anti-FolR1/anti-CD3 bispecific antibody for use in a combination therapy for the treatment of cancer in an individual, wherein the anti-FolR1/anti-CD3 bispecific antibody is used in combination with a a FAP-targeted LTBR agonistic antibody.

In another aspect, the T-cell engaging anti-CD3 bispecific antibody in combination with a FAP-targeted lymphotoxin beta receptor (LTBR) agonistic antibody is for use in a combination therapy for the treatment of cancer in an individual, wherein the T-cell engaging anti-CD3 bispecific antibody and the FAP-targeted LTBR agonistic antibody are administered together in a single composition or administered separately in two or more different compositions. In one aspect, the FAP-targeted LTBR agonistic antibody acts synergistically with the T-cell engaging anti-CD3 bispecific antibody.

In one aspect, the T-cell engaging anti-CD3 bispecific antibody in combination with a FAP-targeted lymphotoxin beta receptor (LTBR) agonistic antibody is for use in a combination therapy for the treatment of cancer in an individual, wherein the FAP-targeted LTBR agonistic antibody comprises at least one antigen binding domain capable of specific binding to LTBR comprising a heavy chain variable region (V_{H}LTBR) comprising (i) CDR-H1 comprising the amino acid sequence of SEQ ID NO:19, (ii) CDR-H2 comprising the amino acid sequence of SEQ ID NO:20, and (iii) CDR-H3 comprising the amino acid sequence of SEQ ID NO:21, and a light chain variable region (V_{L}LTBR) comprising (iv) CDR-L1 comprising the amino acid sequence of SEQ ID NO:22, (v) CDR-L2 comprising the amino acid sequence of SEQ ID NO:23, and (vi) CDR-L3 comprising the amino acid sequence of SEQ ID NO:24. In one aspect, the FAP-targeted LTBR agonistic antibody comprises at least one antigen binding domain capable of specific binding to LTBR comprising a heavy chain variable region (V_{H}LTBR) comprising an amino acid sequence of SEQ ID NO:25 and a light chain variable region (V_{L}LTBR) comprising an amino acid sequence of SEQ ID NO:26. In one particular aspect, the FAP-targeted LTBR agonistic antibody comprises two antigen binding domains capable of specific binding to LTBR each comprising a heavy chain variable region (V_{H}LTBR) comprising an amino acid sequence of SEQ ID NO:25 and a light chain variable region (V_{L}LTBR) comprising an amino acid sequence of SEQ ID NO:26. In one aspect, the FAP-targeted LTBR agonistic antibody comprises an antigen binding domain that specifically binds to FAP comprising
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13, and a light chain variable region (V_{L}FAP) comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16. In one particular aspect, the FAP-targeted LTBR agonistic antibody comprises an antigen binding domain that specifically binds to FAP comprising a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8. In one aspect, the FAP-targeted LTBR agonistic antibody comprises an antigen binding domain that specifically binds to FAP comprising a heavy chain variable region (V_{H}FAP) comprising the amino acid sequence of SEQ ID NO:9 and a light chain variable region (V_{L}FAP) comprising the amino acid sequence of SEQ ID NO:10 or a heavy chain variable region (V_{H}FAP) comprising the amino acid sequence of SEQ ID NO:17 and a light chain variable region (V_{L}FAP) comprising the amino acid sequence of SEQ ID NO:18.

In one aspect, the FAP-targeted LTBR agonistic antibody comprises an IgG Fc domain, specifically an IgG1 Fc domain or an IgG4 Fc domain. In one particular aspect, the FAP-targeted LTBR agonistic antibody comprises a Fc domain with one or more amino acid substitution that reduces binding to an Fc receptor and/or effector function. The crosslinking by a tumor associated antigen FAP makes it possible to avoid unspecific FcγR-mediated crosslinking and thus higher and more efficacious doses of the FAP-targeted LTBR agonistic antibody may be administered in comparison to common LTBR antibodies.

In one aspect, the FAP-targeted LTBR agonistic antibody comprises (a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (b) a second antigen binding domain that specifically binds to LTBR, comprising a heavy chain variable region (V_{H} LTBR) comprising an amino acid sequence of SEQ ID NO:25 and a light chain variable region (V_{L} LTBR) comprising an amino acid sequence of SEQ ID NO:26. In one aspect, the FAP-targeted LTBR agonistic antibody comprises
(a) a first Fab fragment that binds to FAP,
(b) a second and a third Fab fragment that bind to human LTBR, and
(c) a Fc domain of human IgG1 subclass comprising one or more amino acid substitution that reduces the binding affinity of the antigen binding molecule to an Fc receptor and/or effector function,
wherein the first Fab fragment that binds to FAP is fused at its N-terminus to the C-terminus of one of the Fc domain subunits and the second and a third Fab fragment that specifically bind to LTBR are each fused at its C-terminus to the N-terminus of one of the Fc domain subunits.

In one particular aspect, the FAP-targeted LTBR agonistic antibody comprises a first heavy chain comprising the amino acid sequence of SEQ ID NO:51, and a second heavy chain comprising the amino acid sequence of SEQ ID NO:53, two light chains, each comprising the amino acid sequence of SEQ ID NO:52, and one light chain comprising the amino acid sequence of SEQ ID NO:54.

In another aspect, the T-cell engaging anti-CD3 bispecific antibody in combination with a FAP-targeted lymphotoxin beta receptor (LTBR) agonistic antibody is for use in a combination therapy for the treatment of cancer in an individual, wherein the T-cell engaging anti-CD3 bispecific antibody specifically binds to a tumor-associated antigen. In one particular aspect, the tumor-associated antigen is CEA.

In a further aspect, the T-cell engaging anti-CD3 bispecific antibody comprises (i) a heavy chain variable region (V_{H}CD3) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L}CD3) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or (ii) a heavy chain variable region (V_{H}CD3) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L}CD3) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40. In one particular aspect, the T-cell engaging anti-CD3 bispecific antibody comprises a heavy chain variable region (V_{H}CD3) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L}CD3) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32.

In a further aspect, the T-cell engaging anti-CD3 bispecific antibody comprises a heavy chain variable region (V_{H}CD3) comprising the amino acid sequence of SEQ ID NO:33 and a light chain variable region (V_{L}CD3) comprising the amino acid sequence of SEQ ID NO:34 or a heavy chain variable region (V_{H}CD3) comprising the amino acid sequence of SEQ ID NO:41 and a light chain variable region (V_{L}CD3) comprising the amino acid sequence of SEQ ID NO:42. In one aspect, the T-cell engaging anti-CD3 bispecific antibody comprises a heavy chain variable region (V_{H}CD3) comprising the amino acid sequence of SEQ ID NO:33 and a light chain variable region (V_{L}CD3) comprising the amino acid sequence of SEQ ID NO:34.

In one aspect, the T-cell engaging anti-CD3 bispecific antibody is an anti-CEA/anti-CD3 bispecific antibody. In one aspect, the T-cell engaging anti-CD3 bispecific antibody comprises at least one antigen binding domains capable of specific binding to CEA comprising a heavy chain variable region (V_{H}CEA) comprising CDR-H1 sequence of SEQ ID NO:43, CDR-H2 sequence of SEQ ID NO:4, and CDR-H3 sequence of SEQ ID NO:45, and a light chain variable region (V_{L}CEA) comprising CDR-L1 sequence of SEQ ID NO:46, CDR-L2 sequence of SEQ ID NO:47, and CDR-L3 sequence of SEQ ID NO:48. In one aspect, the T-cell engaging anti-CD3 bispecific antibody comprises at least one antigen binding domains capable of specific binding to CEA comprising a heavy chain variable region (V_{H}CEA) comprising the amino acid sequence of SEQ ID NO:49 and a light chain variable region (V_{L}CEA) comprising the amino acid sequence of SEQ ID NO:50. In one particular aspect, the T-cell engaging anti-CD3 bispecific antibody comprises two antigen binding domains capable of specific binding to CEA comprising each a heavy chain variable region (V_{H}CEA) comprising the amino acid sequence of SEQ ID NO:49 and a light chain variable region (V_{L}CEA) comprising the amino acid sequence of SEQ ID NO:50. In one aspect, the the T-cell engaging anti-CD3 bispecific antibody comprises an IgG Fc domain, specifically an IgG1 Fc domain or an IgG4 Fc domain. In one aspect, the T-cell engaging anti-CD3 bispecific antibody comprises an Fc domain comprising one or more amino acid substitutions that reduce binding to an Fc receptor and/or effector function.

In one particular aspect, the T-cell engaging anti-CD3 bispecific antibody comprises a first heavy chain comprising the amino acid sequence of SEQ ID NO:61, and a second heavy chain comprising the amino acid sequence of SEQ ID NO:62, two light chains, each comprising the amino acid sequence of SEQ ID NO:59, and one light chain comprising the amino acid sequence of SEQ ID NO:60.$

In another aspect, the T-cell engaging anti-CD3 bispecific antibody in combination with a FAP-targeted lymphotoxin beta receptor (LTBR) agonistic antibody is for use in a combination therapy for the treatment of cancer in an individual, wherein the T-cell engaging anti-CD3 bispecific antibody specifically binds to the tumor-associated antigen FolR1.

In a particular aspect, the anti-FolR1/anti-CD3 bispecific antibody for use in the combination comprises a first antigen binding domain comprising a heavy chain variable region (V_{H}CD3) comprising the CDR-H1 sequence of SEQ ID NO:108, the CDR-H2 sequence of SEQ ID NO:109, and the CDR-H3 sequence of SEQ ID NO:110; and/or a light chain variable region (V_{L}CD3/FolR1) comprising the CDR-L1 sequence of SEQ ID NO:114, the CDR-L2 sequence of SEQ ID NO:115, and the CDR-L3 sequence of SEQ ID NO:116. More particularly, the anti-FolR1/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:117 and/or a light chain variable region (V_{L}CD3) that is at least 90%, 95%, 96%, 97%, 98%, or 99% identical to the amino acid sequence of SEQ ID NO:119. In a further aspect, the anti-FolR1/anti-CD3 bispecific antibody comprises a heavy chain variable region (V_{H}CD3) comprising the amino acid sequence of SEQ ID NO:117 and/or a light chain variable region (V_{L}CD3) comprising the amino acid sequence of SEQ ID NO:119.

In one aspect, the antibody that specifically binds to CD3 is a full-length antibody. In one aspect, the antibody that specifically binds to CD3 is an antibody of the human IgG class, particularly an antibody of the human IgG1 class. In one aspect, the antibody that specifically binds to CD3 is an antibody fragment, particularly a Fab molecule or a scFv molecule, more particularly a Fab molecule. In a particular aspect, the antibody that specifically binds to CD3 is a crossover Fab molecule wherein the variable domains or the constant domains of the Fab heavy and light chain are exchanged (i.e. replaced by each other). In one aspect, the antibody that specifically binds to CD3 is a humanized antibody.

In another aspect, the anti-FolR1/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. A particularly suitable peptide linker for fusing the Fab light chains of the first and the second Fab molecule to each other is (G₄S)₂. (SEQ ID NO:93). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-FolR1/anti-CD3 bispecific antibody comprises an Fc domain comprising one or more amino acid substitutions that reduce binding to an Fc receptor and/or effector function. In particular, the anti-FolR1/anti-CD3 bispecific antibody comprises an IgG1 Fc domain comprising the amino acid substitutions L234A, L235A and P329G.

In one aspect, the anti-FolR1/anti-CD3 bispecific antibody comprises a first antigen binding domain comprising a heavy chain variable region (V_{H}CD3), a second antigen binding domain comprising a heavy chain variable region (V_{H}FolR1), a third antigen binding domain comprising a heavy chain variable region (V_{H}FolR1) and three times a common light chain variable region.

In one aspect, the first antigen binding domain comprises a heavy chain variable region (V_{H}CD3) comprising the CDR-H1 sequence of SEQ ID NO:108, the CDR-H2 sequence of SEQ ID NO:109, and the CDR-H3 sequence of SEQ ID NO:110; the second antigen binding domain comprises a heavy chain variable region (V_{H}FolR1) comprising the CDR-H1 sequence of SEQ ID NO:111, the CDR-H2 sequence of SEQ ID NO:112, and the CDR-H3 sequence of SEQ ID NO:113; the third antigen binding domain comprises a heavy chain variable region (V_{H}FolR1) comprising the CDR-H1 sequence of SEQ ID NO:111, the CDR-H2 sequence of SEQ ID NO:112, and the CDR-H3 sequence of SEQ ID NO:113; and the common light chains comprise the CDR-L1 sequence of SEQ ID NO:114, the CDR-L2 sequence of SEQ ID NO: 115, and CDR-L3 sequence of SEQ ID NO:116. In another aspect, the first antigen binding domain comprises a heavy chain variable region (V_{H}CD3) comprising the sequence of SEQ ID NO:117; the second antigen binding domain comprises a heavy chain variable region (V_{H}FolR1) comprising the sequence of SEQ ID NO: 118; the third antigen binding domain comprises a heavy chain variable region (V_{H}FolR1) comprising the sequence of SEQ ID NO:118; and the common light chains comprise the sequence of SEQ ID NO:119.

In a particular aspect, the anti-FolR1/anti-CD3 bispecific antibody comprises a polypeptide that is at least 95%, 96%, 97%, 98%, or 99% identical to the sequence of SEQ ID NO:120, a polypeptide that is at least 95%, 96%, 97%, 98%, or 99% identical to the sequence of SEQ ID NO: 121, and three times a polypeptide that is at least 95%, 96%, 97%, 98%, or 99% identical to the sequence of SEQ ID NO: 122.

In a further particular aspect, the anti-FolR1/anti-CD3 bispecific antibody comprises a first heavy chain comprising the amino acid sequence of SEQ ID NO:120, a second heavy chain comprising the amino acid sequence of SEQ ID NO:121 and three times a common light chain of SEQ ID NO: 122 (FolR1 TCB).

In one further aspect, the T-cell engaging anti-CD3 bispecific antibody in combination with a FAP-targeted lymphotoxin beta receptor (LTBR) agonistic antibody is for use in a combination therapy for the treatment of cancer in an individual, wherein the T-cell engaging anti-CD3 bispecific antibody and the FAP-targeted LTBR agonistic antibody are administered intravenously or subcutaneously. In another aspect, the T-cell engaging anti-CD3 bispecific antibody in combination with a FAP-targeted lymphotoxin beta receptor (LTBR) agonistic antibody is for use in a combination therapy for the treatment of cancer in an individual, wherein the T-cell engaging anti-CD3 bispecific antibody is administered concurrently with, prior to, or subsequently to the FAP-targeted LTBR agonistic antibody.

In another aspect, the invention provides a FAP-targeted LTBR agonistic antibody for use in the treatment of a cancer in an individual, wherein the treatment comprises administration of the FAP-targeted LTBR agonistic antibody in combination with a T-cell engaging anti-CD3 bispecific antibody. In another aspect, the invention provides a T-cell engaging anti-CD3 bispecific antibody for use in the treatment of a cancer in an individual, wherein the treatment comprises administration of the T-cell engaging anti-CD3 bispecific antibody in combination with a FAP-targeted LTBR agonistic antibody. In all these aspects, the FAP-targeted LTBR agonistic antibody and/or T-cell engaging anti-CD3 bispecific antibody are as defined hereinbefore.

In a further aspect, provided is the use of a FAP-targeted LTBR agonistic antibody in the manufacture of a medicament for the treatment of cancer in an individual, wherein the treatment comprises administration of the FAP-targeted LTBR agonistic antibody in combination with a T-cell engaging anti-CD3 bispecific antibody. In yet a further aspect, provided is the use of a T-cell engaging anti-CD3 bispecific antibody in the manufacture of a medicament for the treatment of cancer in an individual, wherein the treatment comprises administration of the a T-cell engaging anti-CD3 bispecific antibody in combination with a FAP-targeted LTBR agonistic antibody. In all these aspects, the FAP-targeted LTBR agonistic antibody and/or T-cell engaging anti-CD3 bispecific antibody are as defined hereinbefore.

In another aspect of the invention, provided is a method for treating cancer in an individual comprising administering to the individual an effective amount of a T-cell engaging anti-CD3 bispecific antibody and an effective amount of a FAP-targeted LTBR agonistic antibody. In one aspect, the invention provides a T-cell engaging anti-CD3 bispecific antibody specific for a tumor-associated antigen, in particular an anti-CEA/anti-CD3 bispecific antibody or an anti-FolR1/anti-CD3 bispecific antibody. In one aspect, provided is a T-cell engaging anti-CD3 bispecific antibody specific for a tumor-associated antigen, in particular an anti-CEA/anti-CD3 bispecific antibody or an anti-FolR1/anti-CD3 bispecific antibody, for use in a method for treating or delaying progression of cancer, wherein the anti-CEA/anti-CD3 bispecific antibody comprises a first antigen binding domain that binds to CD3, and a second antigen binding domain that binds to CEA. In particular, the anti-CEA/anti-CD3 bispecific antibody comprises a third antigen binding domain that binds to CEA. In one aspect, provided is an anti-CEA/anti-CD3 bispecific antibody or an anti-FolR1/anti-CD3 bispecific antibody for use in a method for treating or delaying progression of cancer, wherein the first antigen binding domain is a cross-Fab molecule wherein the variable domains or the constant domains of the Fab heavy and light chain are exchanged, and the second and third, if present, antigen binding domain is a conventional Fab molecule. In one further aspect, provided is an anti-CEA/anti-CD3 bispecific antibody or an anti-FolR1/anti-CD3 bispecific antibody for use in a method for treating or delaying progression of cancer, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain. In a further aspect, the anti-CEA/anti-CD3 bispecific antibody or an anti-FolR1/anti-CD3 bispecific antibody for use in a method for treating or delaying progression of cancer comprises an Fc domain comprising one or more amino acid substitutions that reduce binding to an Fc receptor and/or effector function. More particularly, the anti-CEA/anti-CD3 bispecific antibody or anti-FolR1/anti-CD3 bispecific antibody comprises an IgG1 Fc domain comprising the amino acid substitutions L234A, L235A and P329G.

In another aspect, provided is a T-cell engaging anti-CD3 bispecific antibody, in particular an anti-CEA/anti-CD3 bispecific antibody or an anti-FolR1/anti-CD3 bispecific antibody, for use in a method for treating or delaying progression of cancer, wherein the T-cell engaging anti-CD3 bispecific antibody specific for a tumor-associated antigen is used in combination with a FAP-targeted LTBR agonistic antibody and wherein the combination is administered at intervals from about one week to three weeks.

In yet another aspect, the invention provides a kit comprising a first medicament comprising T-cell engaging anti-CD3 bispecific antibody and a second medicament comprising a FAP-targeted LTBR agonistic antibody, and optionally further comprising a package insert comprising instructions for administration of the first medicament in combination with the second medicament for treating cancer in an individual.

### BRIEF DESCRIPTION OF THE DRAWINGS

FIG. 1 shows the study design of an in vivo mouse study to evaluate the efficacy and pharmacodynamic profile of an anti-FAP/anti-LTBR bispecific antibody (mouse surrogate molecule P1AG5459) in combination with an anti-CEA/anti-CD3 bispecific antibody (murine surrogate T cell engager P1AA9604). The timeline describes the treatment and sacrifice timepoints and the table describes the details of the treatment groups, the dosing and the scheduling. White arrows depict administration of P1AG5459 and black arrows depict administration of P1AA9604.

FIG. 2A, FIG. 2B, and FIG. 2C to show that the combination therapy of P1AG5459 and P1AA9604 is the most effective in controlling tumor growth and improving survival/time to event. Mean and standard deviation of percent change of tumor volume from baseline of Vehicle, P1AG5459, P1AA9604 and P1AG5459+P1AA9604 treated mice are plotted in FIG. 2A. Percent change of tumor volume from baseline for each individual mouse at study termination (21 days after tumor cell injection) is plotted as a waterfall plot in FIG. 2B. Kaplan-Meier curves of survival/time to event are plotted in FIG. 2C. In this study an event was defined as reaching a tumor size of 290 mm³. Statistical analysis was performed by Log-Rank test with Bonferroni-Holm correction for multiple comparisons (* p<0.05, ** p<0.01, *** p<0.001, **** p<0.0001).

FIG. 3A and FIG. 3B show that P1AG5459 specifically induces the upregulation of CXCL13 in serum and tumor. The concentration of CXCL13 in serum samples collected 6 days after therapy started is plotted in FIG. 3A. The concentration of CXCL13 in tumor lysates samples collected 6 days after therapy started is plotted in FIG. 3B. Statistical analysis was performed by One-way ANOVA and subsequent multiple comparison testing with Tukey correction (* p<0.05, ** p<0.01, *** p<0.001, **** p<0.0001).

FIG. 4A and FIG. 4B show that the combination therapy induces an increase in the abundance of B cells and CD8⁺ T cells in the tumor at day 6 post treatment start. The number of B220⁺ B cells quantified by histology is plotted in FIG. 4A. The number of CD8⁺ T cells quantified by histology is plotted in FIG. 4B. Statistical analysis was performed by One-way ANOVA and subsequent multiple comparison testing with Tukey correction (* p<0.05, ** p<0.01, *** p<0.001, **** p<0.0001).

FIG. 5 shows representative immunofluorescence images of orthotopic breast cancer tumors collected 6 days after treatment with Vehicle, P1AG5459, P1AA9604 and P1AG5459+P1AA9604. Black arrows indicate pNAD+ high endothelial venules. The representative images show that P1AG5459 specifically induces the differentiation of tumor blood vessels to pNAD+ HEVs.

FIG. 6A and FIG. 6B show a particular FAP-LTBR antibody and a particular anti-CEA/anti-CD3 bispecific antibody as used in the Examples. These molecules are described in more detail in Examples 1 and 2, respectively. The thick black point stands for the knob-into-hole modification. * symbolizes amino acid modifications in the CH1 and CL domain (so-called charged residues). FIG. 6A shows a schematic representation of the 2+1 anti-LTBR/anti-FAP bispecific antibody as human IgG1 PG LALA crossMab with crossed V-domains in both anti-LTBR Fab arms and charges in the CH1/Ck domains of the anti-FAP Fab fragment that is fused to the C-terminus of the Fc domain. In FIG. 6B an exemplary bispecific anti-CEA/anti-CD3 antibody in 2+1 format is shown (named CEA CD3 TCB or CEA TCB).

FIG. 7 shows the study design of an in vivo mouse study to evaluate the efficacy and pharmacodynamic profile of an anti-FAP/anti-LTBR bispecific antibody (P1AG7512) in combination with an anti-FOLR1/anti-CD3 bispecific T cell engager (P1AK1120) in a human breast cancer PDX model in humanized mice. The timeline describes the treatment and sacrifice timepoints and the table describes the details of the treatment groups, the dosing and the scheduling. White arrows depict administration of P1AG7512 and black arrows depict administration of P1AK1120.

FIG. 8A, FIG. 8B and FIG. 8C show that the combination therapy of P1AG7512 and P1AK1120 is the most effective in controlling tumor growth and improving survival/time to event. Median and robust standard deviation of tumor volume of Vehicle, P1AG7512, P1AK1120 and P1AG7512+P1AK1120 treated mice are plotted in FIG. 8A. Percent change of tumor volume from baseline for each individual mouse at study termination (51 days after tumor cell injection) is plotted as a waterfall plot in FIG. 8B. Kaplan-Meier curves of survival/time to event are plotted in FIG. 8C. In this study an event was defined as reaching a tumor size of 240 mm³. Statistical analysis was performed by Log-Rank test with Bonferroni-Holm correction for multiple comparisons (* p<0.05, ** p<0.01).

FIG. 9A and FIG. 9B show that P1AG7512 induces the upregulation of murine CXCL13 but not human CXCL13 in serum of treated mice. The concentration of murine CXCL13 in serum samples collected 8 days after therapy started is plotted in FIG. 9A while the concentration of human CXCL13 in serum samples is shown in FIG. 9B. Statistical analysis was performed by One-way ANOVA and subsequent multiple comparison testing with Tukey correction (* p<0.05, ** p<0.01, *** p<0.001, **** p<0.0001).

FIG. 10A, FIG. 10B, FIG. 10C, FIG. 10D and FIG. 10E show that the combination therapy induces an increase in the abundance of CD8⁺ T cells in general, cytotoxic CD8⁺ GrzB⁺ T cells and high endothelial venules (HEVs) in particular, along with a reduction in regulatory T cells, in the tumor at termination (51 days post tumor inoculation and 23 days post treatment start). All quantifications are derived from 3DIP immunofluorescence. The number of CD8⁺ T cells is plotted in FIG. 10A. The number of regulatory T cells (FoxP3⁺ CD3⁺) is plotted in FIG. 10B. The CD8 to Treg ratio is plotted in FIG. 10C. The percentage of CD8⁺ GrzB⁺ T cells of CD8⁺ T cells is plotted in FIG. 10D. The number of HEVs is plotted in FIG. 10E. Statistical analysis was performed by One-way ANOVA and subsequent multiple comparison testing with Tukey correction (* p<0.05, ** p<0.01, *** p<0.001, **** p<0.0001).

FIG. 11A, FIG. 11B, FIG. 11C and FIG. 11D each show representative 3DIP immunofluorescence images of high endothelial venules in orthotopic breast cancer PDXs collected at termination with Vehicle (FIG. 11A), treatment with only P1AG7512 (FIG. 11B), treatment with P1AK1120 alone (FIG. 11C) and treatment with the combination of P1AG7512 and P1AK1120 (FIG. 11D). The representative images show that the combination of P1AG7512 with P1AK1120 specifically induces the differentiation of tumor blood vessels to pNAD⁺ HEVs, much more than with the P1AG7512 treatment alone.

FIG. 12A and FIG. 12B show representative 3DIP immunofluorescence images of high endothelial venules, CD8⁺ T cells, FoxP3⁺ Tregs and E-cadherin⁺ cancer cells in an orthotopic breast cancer PDXs collected at termination with P1AK1120 treatment and the combination treatment with P1AG7512+P1AK1120. The representative images show that the combination of P1AG7512+P1AK1120 induces the differentiation of tumor blood vessels to pNAD⁺ HEVs and shifts the balance from similar CD8 and Treg numbers towards a tumor microenvironment more enriched with CD8⁺ T cells and less enriched with immunosuppressive Tregs. These are representative images for FIGS. 10A to 10C and FIG. 10E.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the terms “antigen binding molecule” or “antibody” are used interchangeably and refer in their broadest sense to a molecule that specifically binds an antigenic determinant. Examples of antigen binding molecules are antibodies, bi- or multispecific antibodies, antibody fragments and scaffold antigen binding proteins. The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

As used herein, the term “antigen binding domain capable of specific binding to a target cell antigen” or “moiety capable of specific binding to a target cell antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the LTBR agonistic antibody) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565). In particular, the antigen binding domain capable of specific binding to a target cell antigen is an antigen binding domain capable of specific binding to Fibroblast Activation Protein (FAP). In relation to an antibody or fragment thereof, the term “antigen binding domain capable of specific binding to a target cell antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH) of an antibody. In another aspect, the “antigen binding domain capable of specific binding to a target cell antigen” can also be a Fab fragment or a cross-Fab fragment.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antibody is able to specifically bind to at least two distinct antigenic determinants. Typically, a bispecific antibody comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells. A bispecific antibody as described herein can also form part of a multispecific antibody.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region. According to the present invention, the term “Fab fragment” also includes “cross-Fab fragments” or “crossover Fab fragments” as defined below.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab _{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab _{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks.

An “antibody that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more. An “antibody that does not bind to the same epitope” as a reference molecule refers to an antigen binding molecule that does not block binding of the reference molecule to its antigen in a competition assay by 50% or more, and conversely, the reference molecule does not block binding of the antigen binding molecule to its antigen in a competition assay by 50% or more.

The term “antigen binding domain” or “antigen-binding site” refers to the part of an antibody that comprises the area which specifically binds to and is complementary to part or all of an antigen. Where an antigen is large, an antibody may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

As used herein, the term “specific binding” means that the binding is selective for the antigen and can be discriminated from unwanted or non-specific interactions. The ability of an antigen-binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument), and traditional binding assays. In one aspect, the extent of binding of an antigen-binding moiety to an unrelated protein is less than about 10% of the binding of the antigen-binding moiety to the antigen as measured, e.g., by SPR. In certain aspects, an antigen-binding moiety that binds to the antigen, or an antibody comprising that antigen-binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

A “target cell antigen” as used herein refers to an antigenic determinant presented on the surface of a target cell, in particular a target cell in a tumor such as a cancer cell or a cell of the tumor stroma. Thus, the target cell antigen is a tumor-associated antigen. In particular, the “tumor-associated antigen” or TAA is Fibroblast Activation Protein (FAP) or CEA.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:2), or NCBI (www.ncbi.nlm.nih.gov/) RefSeq NP_004451.2. The extracellular domain (ECD) of human FAP extends from amino acid position 26 to 760. The amino acid sequence of an Avi-His-tagged human FAP is shown in SEQ ID NO: 74. The amino acid sequence of mouse FAP is shown in UniProt accession no. P97321 (version 126, SEQ ID NO:75), or NCBI RefSeq NP_032012.1. The extracellular domain (ECD) of mouse FAP extends from amino acid position 26 to 761. SEQ ID NO:76 shows the amino acid of a Avi-His-tagged mouse FAP. Preferably, an anti-FAP binding molecule of the invention binds to the extracellular domain of FAP. Exemplary anti-FAP binding molecules are described in International Patent Application No. WO 2012/020006 A2.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:77). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarstrom S., Semin Cancer Biol. 9(2):67-81 (1999)), making it inaccessible to antibody in the blood stream. In contrast to normal tissue, CEA tends to be expressed over the entire surface of cancerous cells (Hammarstrom S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “FolR1” refers to Folate receptor alpha and has been identified as a potential prognostic and therapeutic target in a number of cancers. It refers to any native FolR1 from any vertebrate source, including mammals such as primates (e.g. humans) non-human primates (e.g. cynomolgus monkeys) and rodents (e.g. mice and rats), unless otherwise indicated. The amino acid sequence of human FolR1 is shown in UniProt accession no. P15328 (SEQ ID NO:123), murine FolR1 has the amino acid sequence of UniProt accession no. P35846 (SEQ ID NO:124) and cynomolgus FolR1 has the amino acid sequence as shown in UniProt accession no. G7PR14 (SEQ ID NO:125). FolR1 is an N-glycosylated protein expressed on plasma membrane of cells. FolR1 has a high affinity for folic acid and for several reduced folic acid derivatives and mediates delivery of the physiological folate, 5-methyltetrahydrofolate, to the interior of cells. FOLR1 is a desirable target for FOLR1-directed cancer therapy as it is overexpressed in vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid. Recent studies have identified that FolR1 expression is particularly high in triple negative breast cancers (Necela et al. PloS One 2015, 10(3), e0127133).

A “T-cell antigen” as used herein refers to an antigenic determinant presented on the surface of a T lymphocyte, particularly a cytotoxic T lymphocyte.

A “T cell engaging therapeutic agent” as used herein refers to a therapeutic agent capable of inducing T cell activation in a subject, particularly a therapeutic agent designed for inducing T-cell activation in a subject. Examples of T cell engaging therapeutic agents include bispecific antibodies that specifically bind an engaging T cell antigen, such as CD3, and a target cell antigen, such as CEA or Folate Receptor.

An “engaging T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an engaging T cell antigen may induce T cell activation by triggering the signaling cascade of the T cell receptor complex. An exemplary engaging T cell antigen is CD3.

The term “CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one aspect, CD3 is human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 78. The amino acid sequence of cynomolgus [Macaca fascicularis] CD3ε is shown in NCBI GenBank no. BAB71849.1. See also SEQ ID NO: 79.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W. H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG1, IgG2, IgG3, IgG4, IgA1, and IgA2. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO: 80). Usually, a segment having the amino acid sequence of EPKSC (SEQ ID NO:81) is following to link the CH1 domain to the hinge region.

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083). In one aspect, the hinge region has the amino acid sequence DKTHTCPXCP (SEQ ID NO: 82), wherein X is either S or P.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. (EU numbering system according to Kabat). In one aspect, a CH2 domain has the amino acid sequence of APELLGGPSV FLFPPKPKDT LMISRTPEVT CVWDVSHEDP EVKFNWYVDG VEVHNAKTKP REEQESTYRW SVLTVLHQDW LNGKEYKCKV SNKALPAPIE KTISKAK (SEQ ID NO: 83). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 according to EU numbering system according to Kabat of an IgG). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO: 84). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “wild-type Fc domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 85 (IgG1, caucasian allotype), SEQ ID NO:86 (IgG1, afroamerican allotype), SEQ ID NO: 87 (IgG2), SEQ ID NO:88 (IgG3) and SEQ ID NO:89 (IgG4). The term “variant (human) Fc domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector function” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492, Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (SEQ ID NO:90, see UniProt accession no. P08637, version 141).

The term “LTBR”, as used herein, refers to any native Lymphotoxin beta receptor (LTBR) from any vertebrate source, including mammals such as primates (e.g. humans) and rodents (e.g., mice and rats), unless otherwise indicated. The term encompasses “full-length,” unprocessed LTBR as well as any form of LTBR that results from processing in the cell. The term also encompasses naturally occurring variants of LTBR, e.g., splice variants or allelic variants. The amino acid sequence of an exemplary human LTBR is shown in SEQ ID NO:1 (Uniprot No. P36941) and the amino acid sequence of an exemplary mouse LTBR is shown in SEQ ID NO: 91 (Uniprot No. B2RRV3). The receptor is expressed on the surface of cells in the parenchyma and stroma of most lymphoid organs but is absent on T- and B-lymphocytes. LTBR can also be referred to as “tumor necrosis factor receptor superfamily member 3 (TNFRSF3). Signaling through LTBR by the LTα/β heterotrimer (LTα1β2) is important during lymphoid development. LTBR is also known to bind the ligand LIGHT (TNFSF14). Whereas LTα1β2 is specific for LTBR, LIGHT also binds to and activates HVEM (TNFRSF14), a receptor expressed on and implicated in the regulation of immune cells.

The term “LTBR agonist” as used herein includes any moiety that agonizes the interaction of LTBR with its ligand. LTBR as used in this context refers preferably to human LTBR, thus the LTBR agonist is preferably an agonist of human LTBR (SEQ ID NO:1). Typically, the moiety will be an agonistic LTBR antibody or antibody fragment.

The terms “anti-LTBR antibody”, “anti-LTBR”, “LTBR antibody and “an antibody that specifically binds to LTBR” refer to an antibody that specifically binds to LTBR with sufficient affinity such that the antibody is useful as a diagnostic and/or therapeutic agent in targeting LTBR. In one aspect, the extent of binding of an anti-LTBR antibody to an unrelated, non-LTBR protein is less than about 10% of the binding of the antibody to LTBR as measured, e.g., by a radioimmunoassay (RIA) or flow cytometry (FACS). In certain embodiments, an antibody that binds to LTBR has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁶ M or less, e.g. from 10⁻⁶ M to 10⁻¹³ M, e.g., from 10⁻⁸ M to 10⁻¹⁰ M).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO:92), GGGGSGGGGS (SEQ ID NO:93), SGGGGSGGGG (SEQ ID NO:94) and GGGGSGGGGSGGGG (SEQ ID NO:95), but also include the sequences GSPGSSSSGS (SEQ ID NO:96), (G4S)₃ (SEQ ID NO:97), (G4S)₄ (SEQ ID NO:98), GSGSGSGS (SEQ ID NO:99), GSGSGNGS (SEQ ID NO:100), GGSGSGSG (SEQ ID NO:101), GGSGSG (SEQ ID NO:102), GGSG (SEQ ID NO:103), GGSGNGSG (SEQ ID NO:104), GGNGSGSG (SEQ ID NO:105) and GGNGSG (SEQ ID NO:106). Peptide linkers of particular interest are (G4S) (SEQ ID NO:92), (G₄S)₂ or GGGGSGGGGS (SEQ ID NO:93), (G4S)₃ (SEQ ID NO:97) and (G4S)₄ (SEQ ID NO:98).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a heavy chain of an antibody and a Fab fragment) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611. Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997), Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

In certain embodiments, amino acid sequence variants of the bispecific antibodies provided herein are contemplated. For example, it may be desirable to improve the binding affinity and/or other biological properties of the bispecific antibodies. Amino acid sequence variants of the bispecific antibodies may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the CDRs and Framework (FRs). Amino acid substitutions may be introduced into the molecule of interest and the products screened for a desired activity, e.g., retained/improved antigen binding, decreased immunogenicity, or improved ADCC or CDC.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more CDRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include bispecific antigen binding molecules of the invention with an N-terminal methionyl residue. Other insertional variants of the molecule include the fusion to the N- or C-terminus to a polypeptide which increases the serum half-life of the bispecific antigen binding molecules.

In certain aspects, the bispecific antibodies provided herein are altered to increase or decrease the extent to which the antibody is glycosylated. Glycosylation variants of the molecules may be conveniently obtained by altering the amino acid sequence such that one or more glycosylation sites is created or removed. Where the bispecific antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the bispecific antibody may be made in order to create variants with certain improved properties. In one aspect, variants of bispecific antigen binding molecules or antibodies of the invention are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). In another aspect, variants of the bispecific antigen binding molecules or antibodies of the invention are provided with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antibodies of the invention, e.g., “thioMAbs,” in which one or more residues of the molecule are substituted with cysteine residues. In particular aspects, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain aspects, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

The term “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or a bispecific antibody” refers to one or more nucleic acid molecules encoding the heavy and light chains (or fragments thereof) of the bispecific antigen binding molecule or antibody, including such nucleic acid molecule(s) in a single vector or separate vectors, and such nucleic acid molecule(s) present at one or more locations in a host cell.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

The term “chemotherapeutic agent” as used herein refers to a chemical compound useful in the treatment of cancer. In one aspect, the chemotherapeutic agent is an antimetabolite. In one aspect, the antimetabolite is selected from the group consisting of Aminopterin, Methotrexate, Pemetrexed, Raltitrexed, Cladribine, Clofarabine, Fludarabine, Mercaptopurine, Pentostatin, Thioguanine, Capecitabine, Cytarabine, Fluorouracil, Floxuridine, and Gemcitabine. In one particular aspect, the antimetabolite is capecitabine or gemcitabine. In another aspect, the antimetabolite is fluorouracil. In one aspect, the chemotherapeutic agent is an agent that affects microtubule formation. In one aspect, the agent that affects microtubule formation is selected from the group consisting of: paclitaxel, docetaxel, vincristine, vinblastine, vindesine, vinorelbine, taxotere, etoposide, and teniposide. In another aspect, the chemotherapeutic agent is an alkylating agent such as cyclophosphamide. In one aspect, the chemotherapeutic agent is a cytotoxic antibiotic such as a topoisomerase II inhibitor. In one aspect, the topoisomerase II inhibitor is doxorubicin.

The combination therapies in accordance with the invention have a synergistic effect. A “synergistic effect” of two compounds is one in which the effect of the combination of the two agents is greater than the sum of their individual effects and is statistically different from the controls and the single drugs. In another embodiment, the combination therapies disclosed herein have an additive effect. An “additive effect” of two compounds is one in which the effect of the combination of the two agents is the sum of their individual effects and is statistically different from either the controls and/or the single drugs.

### Exemplary T-Cell Engaging Anti-CD3 Bispecific Antibodies for Use in the Invention

In one aspect, disclosed herein are anti-CEA/anti-CD3 bispecific antibodies or anti-FolR1/anti-CD3 bispecific antibodies and their use in combination with FAP-targeted LTBR agonistic antibodies, in particular their use in the treatment of cancer, more particularly in the treatment of solid tumors. The anti-CEA/anti-CD3 or anti-FolR1/anti-CD3 bispecific antibodies as used herein are bispecific antibodies comprising a first antigen binding domain that binds to CD3, and a second antigen binding domain that binds to CEA or FolR1.

In one aspect, disclosed herein are anti-CEA/anti-CD3 bispecific antibodies and their use in combination with FAP-targeted LTBR agonistic antibodies, in particular their use in the treatment of cancer, more particularly in the treatment of solid tumors. The anti-CEA/anti-CD3 bispecific antibodies as used herein are bispecific antibodies comprising a first antigen binding domain that binds to CD3, and a second antigen binding domain that binds to CEA.

Thus, the anti-CEA/anti-CD3 bispecific antibody as used herein comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

In a particular aspect, the anti-CEA/anti-CD3 bispecific antibody for use in the combination comprises a first antigen binding domain comprising a heavy chain variable region (V_{H}CD3) comprising CDR-H1 sequence of SEQ ID NO:27, CDR-H2 sequence of SEQ ID NO:28, and CDR-H3 sequence of SEQ ID NO:29; and/or a light chain variable region (V_{L}CD3) comprising CDR-L1 sequence of SEQ ID NO:30, CDR-L2 sequence of SEQ ID NO:31, and CDR-L3 sequence of SEQ ID NO:32. More particularly, the anti-CEA/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:33 and/or a light chain variable region (V_{L}CD3) that is at least 90%, 95%, 96%, 97%, 98%, or 99% identical to the amino acid sequence of SEQ ID NO:34. In a further aspect, the anti-CEA/anti-CD3 bispecific antibody comprises a heavy chain variable region (V_{H}CD3) comprising the amino acid sequence of SEQ ID NO:33 and/or a light chain variable region (V_{L}CD3) comprising the amino acid sequence of SEQ ID NO:34.

In another aspect, the anti-CEA/anti-CD3 bispecific antibody for use in the combination comprises a first antigen binding domain comprising a heavy chain variable region (V_{H}CD3) comprising CDR-H1 sequence of SEQ ID NO:35, CDR-H2 sequence of SEQ ID NO:36, and CDR-H3 sequence of SEQ ID NO:37; and/or a light chain variable region (V_{L}CD3) comprising CDR-L1 sequence of SEQ ID NO:38, CDR-L2 sequence of SEQ ID NO:39, and CDR-L3 sequence of SEQ ID NO:40. More particularly, the anti-CEA/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:41 and/or a light chain variable region (V_{L}CD3) that is at least 90%, 95%, 96%, 97%, 98%, or 99% identical to the amino acid sequence of SEQ ID NO:42. In a further aspect, the anti-CEA/anti-CD3 bispecific antibody comprises a heavy chain variable region (V_{H}CD3) comprising the amino acid sequence of SEQ ID NO:41 and/or a light chain variable region (V_{L}CD3) comprising the amino acid sequence of SEQ ID NO:42.

In one aspect, the antibody that specifically binds to CD3 is a full-length antibody. In one aspect, the antibody that specifically binds to CD3 is an antibody of the human IgG class, particularly an antibody of the human IgG₁ class. In one aspect, the antibody that specifically binds to CD3 is an antibody fragment, particularly a Fab molecule or a scFv molecule, more particularly a Fab molecule. In a particular aspect, the antibody that specifically binds to CD3 is a crossover Fab molecule wherein the variable domains or the constant domains of the Fab heavy and light chain are exchanged (i.e. replaced by each other). In one aspect, the antibody that specifically binds to CD3 is a humanized antibody.

In another aspect, the anti-CEA/anti-CD3 bispecific antibody comprises a second antigen binding domain comprising a heavy chain variable region (V_{H}CEA) comprising CDR-H1 sequence of SEQ ID NO:43, CDR-H2 sequence of SEQ ID NO:4, and CDR-H3 sequence of SEQ ID NO:45, and/or a light chain variable region (V_{L}CEA) comprising CDR-L1 sequence of SEQ ID NO:46, CDR-L2 sequence of SEQ ID NO:47, and CDR-L3 sequence of SEQ ID NO:48.

More particularly, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:49 and/or a light chain variable region (V_{L}CEA) that is at least 90%, 95%, 96%, 97%, 98%, or 99% identical to the amino acid sequence of SEQ ID NO:50. In a further aspect, the anti-CEA/anti-CD3 bispecific comprises a second antigen binding domain comprising a heavy chain variable region (V_{H}CEA) comprising the amino acid sequence of SEQ ID NO:49 and/or a light chain variable region (V_{L}CEA) comprising the amino acid sequence of SEQ ID NO:50.

In another particular aspect, the anti-CEA/anti-CD3 bispecific antibody comprises a third antigen binding domain that binds to CEA. In particular, the anti-CEA/anti-CD3 bispecific antibody comprises a third antigen binding domain comprising a heavy chain variable region (V_{H}CEA) comprising CDR-H1 sequence of SEQ ID NO:43, CDR-H2 sequence of SEQ ID NO:4, and CDR-H3 sequence of SEQ ID NO:45, and/or a light chain variable region (V_{L}CEA) comprising CDR-L1 sequence of SEQ ID NO:46, CDR-L2 sequence of SEQ ID NO:47, and CDR-L3 sequence of SEQ ID NO:48. More particularly, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:49 and/or a light chain variable region (V_{L}CEA) that is at least 90%, 95%, 96%, 97%, 98%, or 99% identical to the amino acid sequence of SEQ ID NO:50. In a further aspect, the anti-CEA/anti-CD3 bispecific comprises a third antigen binding domain comprising a heavy chain variable region (V_{H}CEA) comprising the amino acid sequence of SEQ ID NO:49 and/or a light chain variable region (V_{L}CEA) comprising the amino acid sequence of SEQ ID NO:50. In a further aspect, the anti-CEA/anti-CD3 bispecific antibody is bispecific antibody, wherein the first antigen binding domain is a cross-Fab molecule wherein the variable domains or the constant domains of the Fab heavy and light chain are exchanged, and the second and third, if present, antigen binding domain is a conventional Fab molecule.

In another aspect, the anti-CEA/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. A particularly suitable peptide linker for fusing the Fab light chains of the first and the second Fab molecule to each other is (G₄S)₂. (SEQ ID NO:93). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-CEA/anti-CD3 bispecific antibody comprises an Fc domain comprising one or more amino acid substitutions that reduce binding to an Fc receptor and/or effector function. In particular, the anti-CEA/anti-CD3 bispecific antibody comprises an IgG1 Fc domain comprising the amino acid substitutions L234A, L235A and P329G.

In a particular aspect, the anti-CEA/anti-CD3 bispecific antibody comprises a polypeptide that is at least 95%, 96%, 97%, 98%, or 99% identical to the sequence of SEQ ID NO: 60, two times a polypeptide that is at least 95%, 96%, 97%, 98%, or 99% identical to the sequence of SEQ ID NO: 59, a polypeptide that is at least 95%, 96%, 97%, 98%, or 99% identical to the sequence of SEQ ID NO: 61, and a polypeptide that is at least 95%, 96%, 97%, 98%, or 99% identical to the sequence of SEQ ID NO: 62. In a further particular embodiment, the bispecific antibody comprises a polypeptide sequence of SEQ ID NO: 60, two times a polypeptide sequence of SEQ ID NO: 59, a polypeptide sequence of SEQ ID NO: 61 and a polypeptide sequence of SEQ ID NO: 62 (CEA TCB).

In another aspect, the anti-CEA/anti-CD3 bispecific antibody comprises a polypeptide that is at least 95%, 96%, 97%, 98%, or 99% identical to the sequence of SEQ ID NO:64, two times a polypeptide that is at least 95%, 96%, 97%, 98%, or 99% identical to the sequence of SEQ ID NO:63, a polypeptide that is at least 95%, 96%, 97%, 98%, or 99% identical to the sequence of SEQ ID NO:65, and a polypeptide that is at least 95%, 96%, 97%, 98%, or 99% identical to the sequence of SEQ ID NO:66. In a further particular embodiment, the bispecific antibody comprises a polypeptide sequence of SEQ ID NO:64, two times a polypeptide sequence of SEQ ID NO:63, a polypeptide sequence of SEQ ID NO:65 and a polypeptide sequence of SEQ ID NO:66 (CEA TCB murine surrogate).

Particular bispecific antibodies are described in PCT publication no. WO 2014/131712 A1.

In a further aspect, the anti-CEA/anti-CD3 bispecific antibody may also comprise a bispecific T cell engager (BiTE®). In a further aspect, the anti-CEA/anti-CD3 bispecific antibody is a bispecific antibody as described in WO 2007/071426 or WO 2014/131712. In another aspect, the bispecific antibody is MEDI565.

Provided herein are also anti-FolR1/anti-CD3 bispecific antibodies and their use in combination with FAP-targeted LTBR agonistic antibodies, in particular to their use in a method for treating or delaying progression of cancer, more particularly for treating or delaying progression of solid tumors. The anti-FolR1/anti-CD3 bispecific antibodies as used herein are bispecific antibodies comprising a first antigen binding domain that binds to CD3, and a second antigen binding domain that binds to FolR1. In a particular, the anti-FolR1/anti-CD3 bispecific antibodies as used herein comprise a third antigen binding domain that binds to FolR1.

In a particular aspect, the anti-FolR1/anti-CD3 bispecific antibody for use in the combination comprises a first antigen binding domain comprising a heavy chain variable region (V_{H}CD3) comprising the CDR-H1 sequence of SEQ ID NO:108, the CDR-H2 sequence of SEQ ID NO:109, and the CDR-H3 sequence of SEQ ID NO:110; and/or a light chain variable region (V_{L}CD3/FolR1) comprising the CDR-L1 sequence of SEQ ID NO:114, the CDR-L2 sequence of SEQ ID NO:115, and the CDR-L3 sequence of SEQ ID NO:116. More particularly, the anti-FolR1/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:117 and/or a light chain variable region (V_{L}CD3) that is at least 90%, 95%, 96%, 97%, 98%, or 99% identical to the amino acid sequence of SEQ ID NO:119. In a further aspect, the anti-FolR1/anti-CD3 bispecific antibody comprises a heavy chain variable region (V_{H}CD3) comprising the amino acid sequence of SEQ ID NO:117 and/or a light chain variable region (V_{L}CD3) comprising the amino acid sequence of SEQ ID NO:119.

In one aspect, the antibody that specifically binds to CD3 is a full-length antibody. In one aspect, the antibody that specifically binds to CD3 is an antibody of the human IgG class, particularly an antibody of the human IgG1 class. In one aspect, the antibody that specifically binds to CD3 is an antibody fragment, particularly a Fab molecule or a scFv molecule, more particularly a Fab molecule. In a particular aspect, the antibody that specifically binds to CD3 is a crossover Fab molecule wherein the variable domains or the constant domains of the Fab heavy and light chain are exchanged (i.e. replaced by each other). In one aspect, the antibody that specifically binds to CD3 is a humanized antibody.

In another aspect, the anti-FolR1/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. A particularly suitable peptide linker for fusing the Fab light chains of the first and the second Fab molecule to each other is (G₄S)₂. (SEQ ID NO:93). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-FolR1/anti-CD3 bispecific antibody comprises an Fc domain comprising one or more amino acid substitutions that reduce binding to an Fc receptor and/or effector function. In particular, the anti-FolR1/anti-CD3 bispecific antibody comprises an IgG1 Fc domain comprising the amino acid substitutions L234A, L235A and P329G.

In one aspect, the anti-FolR1/anti-CD3 bispecific antibody comprises a first antigen binding domain comprising a heavy chain variable region (V_{H}CD3), a second antigen binding domain comprising a heavy chain variable region (V_{H}FolR1), a third antigen binding domain comprising a heavy chain variable region (V_{H}FolR1) and three times a common light chain variable region.

In one aspect, the first antigen binding domain comprises a heavy chain variable region (V_{H}CD3) comprising the CDR-H1 sequence of SEQ ID NO:108, the CDR-H2 sequence of SEQ ID NO:109, and the CDR-H3 sequence of SEQ ID NO:110; the second antigen binding domain comprises a heavy chain variable region (V_{H}FolR1) comprising the CDR-H1 sequence of SEQ ID NO:111, the CDR-H2 sequence of SEQ ID NO:112, and the CDR-H3 sequence of SEQ ID NO:113; the third antigen binding domain comprises a heavy chain variable region (V_{H}FolR1) comprising the CDR-H1 sequence of SEQ ID NO:111, the CDR-H2 sequence of SEQ ID NO:112, and the CDR-H3 sequence of SEQ ID NO:113; and the common light chains comprise the CDR-L1 sequence of SEQ ID NO:114, the CDR-L2 sequence of SEQ ID NO: 115, and CDR-L3 sequence of SEQ ID NO:116. In another aspect, the first antigen binding domain comprises a heavy chain variable region (V_{H}CD3) comprising the sequence of SEQ ID NO:117; the second antigen binding domain comprises a heavy chain variable region (V_{H}FolR1) comprising the sequence of SEQ ID NO: 118; the third antigen binding domain comprises a heavy chain variable region (V_{H}FolR1) comprising the sequence of SEQ ID NO:118; and the common light chains comprise the sequence of SEQ ID NO:119.

In a particular aspect, the anti-FolR1/anti-CD3 bispecific antibody comprises a polypeptide that is at least 95%, 96%, 97%, 98%, or 99% identical to the sequence of SEQ ID NO:120, a polypeptide that is at least 95%, 96%, 97%, 98%, or 99% identical to the sequence of SEQ ID NO: 121, and three times a polypeptide that is at least 95%, 96%, 97%, 98%, or 99% identical to the sequence of SEQ ID NO: 122.

In a further particular aspect, the anti-FolR1/anti-CD3 bispecific antibody comprises a first heavy chain comprising the amino acid sequence of SEQ ID NO:120, a second heavy chain comprising the amino acid sequence of SEQ ID NO:121 and three times a common light chain of SEQ ID NO: 122 (FolR1 TCB).

### Exemplary FAP-Targeted LTBR Agonistic Antibodies for Use in the Invention

In particular, the FAP-targeted LTBR agonistic antibodies as used in combination with the T-cell engaging anti-CD3 bispecific antibody are molecules comprising agonistic lymphotoxin beta receptor (LTBR) antibodies. In particular, the FAP-targeted LTBR agonistic antibodies comprise an agonistic hu LTBR antibody, more particularly an agonistic hu LTBR antibody that is able to bind to human LTBR, cynomolgus LTBR and murine LTBR.

The agonistic LTBR antibodies as described herein require cross-linking for their agonistic activity to activate human LTBR, meaning that they can only stimulate LTBR via a cross-linking dependent mechanism. A “cross linking dependent mechanism” could be for example a Fc cross linking dependent mechanism wherein the antibody has to bind both LTBR and an Fc receptor in order to stimulate LTBR. As such, the antibody has to be capable of binding both LTBR and an Fc receptor. If the antibody would be crosslinking-independent, it could stimulate LTBR in the absence of binding to an Fc receptor. This could lead to widespread LTBR activation in the human body and serious safety issues associated therewith.

The agonistic LTBR antibodies as described herein also require cross-linking for their agonistic activity to induce ICAM upregulation in human umbilical vein endothelial cells or cancer associated fibroblasts (CAFs). Agonistic LTBR antibodies as described herein are also able to inhibit the interaction between human LTBR and its human ligands lymphotoxin α1β2 and LIGHT, i.e. they compete for binding to hu LTBR with human LIGHT, i.e a natural ligand of LTBR comprising the amino acid sequence of SEQ ID NO:107.

In one aspect, the FAP-targeted LTBR agonistic antibody as disclosed herein comprises at least one antigen binding domain capable of specific binding to LTBR comprising a heavy chain variable region (V_{H}LTBR) comprising (i) CDR-H1 comprising the amino acid sequence of SEQ ID NO:19, (ii) CDR-H2 comprising the amino acid sequence of SEQ ID NO:20, and (iii) CDR-H3 comprising the amino acid sequence of SEQ ID NO:21, and a light chain variable region (V_{L}LTBR) comprising (iv) CDR-L1 comprising the amino acid sequence of SEQ ID NO:22, (v) CDR-L2 comprising the amino acid sequence of SEQ ID NO:23, and (vi) CDR-L3 comprising the amino acid sequence of SEQ ID NO:24.

In a further aspect, the FAP-targeted LTBR agonistic antibody as disclosed herein comprises at least one antigen binding domain capable of specific binding to LTBR comprising a heavy chain variable region (V_{H}LTBR) that is at least 90%, 95%, 96%, 97%, 98%, or 99% identical to the amino acid sequence of SEQ ID NO:25 and/or a light chain variable region (V_{L}LTBR) that is at least 90%, 95%, 96%, 97%, 98%, or 99% identical to the amino acid sequence of SEQ ID NO:26. In a further aspect, the FAP-targeted LTBR agonistic antibody comprises a heavy chain variable region (V_{H}LTBR) comprising the amino acid sequence of SEQ ID NO:25 and/or a light chain variable region (V_{L}CD3) comprising the amino acid sequence of SEQ ID NO:26.

In one aspect, the FAP-targeted LTBR agonistic antibody comprises an antigen binding domain that specifically binds to FAP comprising
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24.

In one aspect, the FAP-targeted LTBR agonistic antibody as disclosed herein comprises an antigen binding domain capable of specific binding to FAP comprising a heavy chain variable region (V_{H}FAP) that is at least 90%, 95%, 96%, 97%, 98%, or 99% identical to the amino acid sequence of SEQ ID NO:9 and/or a light chain variable region (V_{L}FAP) that is at least 90%, 95%, 96%, 97%, 98%, or 99% identical to the amino acid sequence of SEQ ID NO:10. In a further aspect, the FAP-targeted LTBR agonistic antibody comprises a heavy chain variable region (V_{H}FAP) comprising the amino acid sequence of SEQ ID NO:9 and/or a light chain variable region (V_{L}FAP) comprising the amino acid sequence of SEQ ID NO:10.

In another aspect, the FAP-targeted LTBR agonistic antibody as disclosed herein comprises an antigen binding domain capable of specific binding to FAP comprising a heavy chain variable region (V_{H}FAP) that is at least 90%, 95%, 96%, 97%, 98%, or 99% identical to the amino acid sequence of SEQ ID NO:17 and/or a light chain variable region (V_{L}FAP) that is at least 90%, 95%, 96%, 97%, 98%, or 99% identical to the amino acid sequence of SEQ ID NO:18. In a further aspect, the FAP-targeted LTBR agonistic antibody comprises a heavy chain variable region (V_{H}FAP) comprising the amino acid sequence of SEQ ID NO:17 and/or a light chain variable region (V_{L}FAP) comprising the amino acid sequence of SEQ ID NO:18.

In one aspect, provided is a FAP-targeted LTBR agonistic antibody, comprising
(a) a first antigen binding domain hat specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, or
a heavy chain variable region (V_{H}FAP) comprising the amino acid sequence of SEQ ID NO:17 and a light chain variable region (V_{L}FAP) comprising the amino acid sequence of SEQ ID NO:18,
(b) a second antigen binding domain that specifically binds to lymphotoxin beta receptor (LTBR), comprising a heavy chain variable region (V_{H} LTBR) comprising the amino acid sequence of SEQ ID NO:25 and a light chain variable region (V_{L} LTBR) comprising the amino acid sequence of SEQ ID NO:26, and
(c) a Fc domain composed of a first and a second subunit and comprising one or more amino acid substitution that reduces the binding affinity of the antigen binding molecule to an Fc receptor and/or effector function.

In one aspect, provided is a FAP-targeted LTBR agonistic antibody, comprising
(a) a first antigen binding domain hat specifically binds to FAP, comprising
a heavy chain variable region (V_{H}FAP) comprising the amino acid sequence of SEQ ID NO:9 and a light chain variable region (V_{L}FAP) comprising the amino acid sequence of SEQ ID NO:10, or
a heavy chain variable region (V_{H}FAP) comprising the amino acid sequence of SEQ ID NO:17 and a light chain variable region (V_{L}FAP) comprising the amino acid sequence of SEQ ID NO:18,
(b) a second and a third antigen binding domain that specifically binds to lymphotoxin beta receptor (LTBR), comprising a heavy chain variable region (V_{H} LTBR) comprising the amino acid sequence of SEQ ID NO:25 and a light chain variable region (V_{L} LTBR) comprising the amino acid sequence of SEQ ID NO:26, and
(c) a Fc domain composed of a first and a second subunit and comprising one or more amino acid substitution that reduces the binding affinity of the antigen binding molecule to an Fc receptor and/or effector function.

In one particular aspect, provided is a FAP-targeted LTBR agonistic antibody which comprises (i) a first antigen binding domain capable of specific binding to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (ii) a second and third antigen binding domain capable of specific binding to LTBR, comprising a heavy chain variable region (V_{H}LTBR) comprising an amino acid sequence of SEQ ID NO:25 and a light chain variable region (V_{L}LTBR) comprising an amino acid sequence of SEQ ID NO:26.

In another aspect, the FAP-targeted LTBR agonistic antibody comprises a Fc domain composed of a first and a second subunit capable of stable association. In one aspect, the FAP-targeted LTBR agonistic antibody comprises an IgG Fc domain, specifically an IgG1 Fc domain or an IgG4 Fc domain. Particularly, the FAP-targeted LTBR agonistic antibody comprises a Fc domain that comprises one or more amino acid substitution that reduces binding to an Fc receptor and/or effector function. In a particular aspect, the FAP-targeted LTBR agonistic antibody comprises an IgG1 Fc domain comprising the amino acid substitutions L234A, L235A and P329G.

In one aspect, provided is a FAP-targeted LTBR agonistic antibody, comprising a first antigen binding domain that specifically binds to Fibroblast Activation Protein (FAP), a second and a third antigen binding domain that specifically bind to lymphotoxin beta receptor (LTBR), and a Fc domain composed of a first and a second subunit and comprising one or more amino acid substitution that reduces the binding affinity of the antibody to an Fc receptor and/or effector function, wherein the bispecific antibody comprises
(i) two light chains and two heavy chain of a full length antibody which specifically binds to LTBR, and
(ii) a Fab fragment or crossFab fragment that specifically binds to FAP is connected via a peptide linker to one of the C-termini of the two heavy chains.

In another aspect, provided is a FAP-targeted LTBR agonistic antibody, comprising a first antigen binding domain that specifically binds to Fibroblast Activation Protein (FAP), a second antigen binding domain that specifically binds to lymphotoxin beta receptor (LTBR), and a Fc domain composed of a first and a second subunit and comprising one or more amino acid substitution that reduces the binding affinity of the antibody to an Fc receptor and/or effector function, wherein the bispecific antibody comprises
(i) a first light chain and a first heavy chain of a full length antibody which specifically binds to FAP, and
(ii) a second (modified) light chain and a second (modified) heavy chain of a full length antibody which specifically binds to LTBR, wherein a Fab fragment or CrossFab fragment that specifically binds to LTBR is connected via a peptide linker to the C-terminus of the second heavy chain.

Thus, provided is a bispecific antigen binding molecule, wherein the bispecific antigen binding molecule has bivalent binding to LTBR and monovalent binding to FAP.

In one particular aspect, provided is a FAP-targeted LTBR agonistic antibody comprising a first heavy chain comprising the amino acid sequence of SEQ ID NO:51, and a second heavy chain comprising the amino acid sequence of SEQ ID NO:53, two light chains, each comprising the amino acid sequence of SEQ ID NO:52, and one light chain comprising the amino acid sequence of SEQ ID NO:54.

In another aspect, provided is a FAP-targeted LTBR agonistic antibody comprising a first heavy chain comprising the amino acid sequence of SEQ ID NO:55, and a second heavy chain comprising the amino acid sequence of SEQ ID NO:57, two light chains, each comprising the amino acid sequence of SEQ ID NO:56, and one light chain comprising the amino acid sequence of SEQ ID NO:58.

### Preparation of Bispecific Antibodies for Use in the Invention

In certain aspects, the therapeutic agents used in the combination comprise multispecific antibodies, e.g. bispecific antibodies. Multispecific antibodies are monoclonal antibodies that have binding specificities for at least two different sites. In certain aspects, the binding specificities are for different antigens. In certain aspects, the binding specificities are for different epitopes on the same antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking of two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991). “Crossmab” antibodies are also included herein (see e.g. WO 2009/080251, WO 2009/080252, WO2009/080253, or WO2009/080254). Another technique for making bispecific antibody fragments is the “bispecific T cell engager” or BiTE® approach (see, e.g., WO2004/106381, WO2005/061547, WO2007/042261, and WO2008/119567). This approach utilizes two antibody variable domains arranged on a single polypeptide. For example, a single polypeptide chain includes two single chain Fv (scFv) fragments, each having a variable heavy chain (VH) and a variable light chain (VL) domain separated by a polypeptide linker of a length sufficient to allow intramolecular association between the two domains. This single polypeptide further includes a polypeptide spacer sequence between the two scFv fragments. Each scFv recognizes a different epitope, and these epitopes may be specific for different cell types, such that cells of two different cell types are brought into close proximity or tethered when each scFv is engaged with its cognate epitope. One particular embodiment of this approach includes a scFv recognizing a cell-surface antigen expressed by an immune cell, e.g., a CD3 polypeptide on a T cell, linked to another scFv that recognizes a cell-surface antigen expressed by a target cell, such as a malignant or tumor cell. As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In one aspect, the bispecific antibodies used in the invention are composed of a single polypeptide chain comprising two single chain FV fragments (scFV) fused to each other by a peptide linker.

Fc Domain Modifications Reducing Fc Receptor Binding and/or Effector Function

The Fc domain of the bispecific antibodies used herein consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies to cells expressing Fc receptors rather than to the preferred antigen-bearing cells. Accordingly, in particular aspects the Fc domain of the bispecific antibodies exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 Fc domain.

In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an engaging human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. Provided herein are bispecific antibodies, wherein the Fc domain comprises one or more amino acid substitution that reduces binding to an Fc receptor, in particular towards Fcγ receptor. In one aspect, the Fc domain of the bispecific antibodies comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided herein are bispecific antibodies which comprise an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or cell engaging antibodies comprising an Fc domain for Fc receptors may be evaluated using cell lines known to express particular Fc receptors, such as human NK cells expressing FcγIIIa receptor.

Effector function of an Fc domain, or bispecific antibodies comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some aspects, binding of the Fc domain to a complement component, specifically to C1q, is reduced. Accordingly, wherein the Fc domain is engineered to have reduced effector function, said reduced effector function includes reduced CDC. C1q binding assays may be carried out to determine whether the bispecific antibodies of the invention is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

### Fc Domain Modifications Promoting Heterodimerization

The bispecific antibodies described herein comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific agonistic LTBR antibodies in recombinant production, it will thus be advantageous to introduce in the Fc domain of the bispecific antibodies a modification promoting the association of the desired polypeptides.

Accordingly, in particular aspects provided is a bispecific antibody for use herein comprising (a) a first antigen binding domain that specifically binds to Fibroblast Activation Protein (FAP), (b) a second antigen binding domain that specifically binds to lymphotoxin beta receptor (LTBR), and (c) a Fc domain composed of a first and a second subunit and comprising one or more amino acid substitution that reduces the binding affinity of the antigen binding molecule to an Fc receptor and/or effector function., wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, provided is a FAP-targeted LTBR agonistic antibody comprising (a) at least one antigen binding domain capable of specific binding to LTBR, (b) at least one antigen binding domain capable of specific binding to a target cell antigen, and (c) a Fc domain composed of a first and a second subunit capable of stable association, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antibodies as reported herein can be a complete C-terminus ending with the amino acid residues PGK. The C-terminus of the heavy chain can be a shortened C-terminus in which one or two of the C terminal amino acid residues have been removed. In one particular aspect, the C-terminus of the heavy chain is a shortened C-terminus ending PG. In one particular aspect, the C-terminus of the heavy chain is a shortened C-terminus ending P. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one aspect of all aspects as reported herein, a bispecific agonistic LTBR antibody comprising a heavy chain including a C-terminal CH3 domain, as specified herein, comprises a C-terminal glycine residue (G446, numbering according to Kabat EU index).

### Modifications in the Fab Domains

In one aspect, provided is a FAP-targeted LTBR agonistic antibody comprising (a) a first Fab fragment capable of specific binding to FAP, (b) a second Fab fragment capable of specific binding to LTBR, and (c) a Fc domain composed of a first and a second subunit, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, provided is a FAP-targeted LTBR agonistic antibody comprising (a) a first Fab fragment capable of specific binding to FAP, (b) a second Fab fragment capable of specific binding to LTBR, and (c) a Fc domain composed of a first and a second subunit capable of stable association, wherein in one of the Fab fragments the variable domains VL and VH are replaced by each other so that the VH domain is part of the light chain and the VL domain is part of the heavy chain. More particularly, in the second Fab fragment capable of specific binding to LTBR the variable domains VL and VH are replaced by each other so that the VH domain is part of the light chain and the VL domain is part of the heavy chain.

In another aspect, provided is a FAP-targeted LTBR agonistic antibody comprising (a) a first Fab fragment capable of specific binding to FAP, wherein the constant domains CL and CH1 are replaced by each other so that the CH1 domain is part of the light chain and the CL domain is part of the heavy chain, and (b) a second Fab fragment capable of specific binding to LTBR. Such a molecule provides monovalent binding to both LTBR and FAP.

In another aspect, provided is a FAP-targeted LTBR agonistic antibody comprising (a) a first Fab fragment that specifically binds to Fibroblast Activation Protein (FAP), (b) a second and a third Fab fragment that specifically bind to lymphotoxin beta receptor (LTBR), and (c) a Fc domain composed of a first and a second subunit and comprising one or more amino acid substitution that reduces the binding affinity of the antigen binding molecule to an Fc receptor and/or effector function, wherein in the second and third Fab fragment that specifically to LTBR, the variable domains VL and VH are replaced by each other so that the VH domain is part of the light chain and the VL domain is part of the heavy chain.

In another aspect, and to further improve correct pairing, the FAP-targeted LTBR agonistic antibody comprises (a) a first Fab fragment capable of specific binding to LTBR, (b) a second Fab fragment capable of specific binding to FAP, and (c) a Fc domain composed of a first and a second subunit capable of stable association, can contain different charged amino acid substitutions (so-called “charged residues”). These modifications are introduced in the crossed or non-crossed CH1 and CL domains. In a particular aspect, the invention relates to a bispecific antigen binding molecule, wherein in one of CL domains the amino acid at position 123 (EU numbering) has been replaced by arginine (R) and/or wherein the amino acid at position 124 (EU numbering) has been substituted by lysine (K) and wherein in one of the CH1 domains the amino acids at position 147 (EU numbering) and/or at position 213 (EU numbering) have been substituted by glutamic acid (E).

### Polynucleotides

Further provided are isolated polynucleotides encoding a bispecific antibody as described herein or a fragment thereof.

The isolated polynucleotides encoding a bispecific antibody may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes a polypeptide comprised in the bispecific antibody as described herein.

In one aspect, provided is an isolated polynucleotide encoding a FAP-targeted LTBR agonistic antibody, comprising (a) a first antigen binding domain that specifically binds to Fibroblast Activation Protein (FAP), (b) a second antigen binding domain that specifically binds to lymphotoxin beta receptor (LTBR), and (c) a Fc domain composed of a first and a second subunit and comprising one or more amino acid substitution that reduces the binding affinity of the antigen binding molecule to an Fc receptor and/or effector function.

In certain aspects, the polynucleotide or nucleic acid is DNA. In other embodiments, a polynucleotide of the present invention is RNA, for example, in the form of messenger RNA (mRNA). RNA as described herein may be single stranded or double stranded.

### Pharmaceutical Compositions, Formulations and Routes of Administration

In a further aspect, the invention provides pharmaceutical compositions comprising the T-cell engaging anti-CD3 bispecific antibody specific for a tumor-associated antigen, in particular an anti-CEA/anti-CD3 bispecific antibody or an anti-FolR1/anti-CD3 bispecific antibody, and FAP-targeted LTBR agonistic antibodies provided herein, e.g., for use in any of the below therapeutic methods. In one embodiment, a pharmaceutical composition comprises an antibody provided herein and at least one pharmaceutically acceptable excipient. In another embodiment, a pharmaceutical composition comprises an antibody provided herein and at least one additional therapeutic agent, e.g., as described below. In another aspect, provided are pharmaceutical compositions comprising the T-cell engaging anti-CD3 bispecific antibody specific for a tumor-associated antigen, in particular an anti-CEA/anti-CD3 bispecific antibody or an anti-FolR1/anti-CD3 bispecific antibody, and FAP-targeted LTBR agonistic antibodies provided herein.

Pharmaceutical compositions as disclosed herein comprise a therapeutically effective amount of one or more bispecific antibodies dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one bispecific agonistic LTBR antibody and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intra-lesional, intravenous, intra-arterial, intramuscular, intrathecal or intraperitoneal injection. For injection, the bispecific antibodies may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the bispecific antibodies may be in powder form for constitution with a suitable vehicle, e.g., sterile pyrogen-free water, before use. Sterile injectable solutions are prepared by incorporating the bispecific antibodies in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular aspects, prolonged absorption of an injectable composition can be brought about by the use in the compositions of agents delaying absorption, such as, for example, aluminum monostearate, gelatin or combinations thereof.

In addition to the compositions described previously, the bispecific antibodies may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as emulsion in an acceptable oil) or ion exchange resins, or as sparingly soluble derivatives, for example, as a sparingly soluble salt.

Pharmaceutical compositions comprising the bispecific antibodies disclosed herein may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antibodies may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The compositions herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

### Administration of the T-Cell Engaging Anti-CD3 Bispecific Antibody and the FAP-Targeted LTBR Agonistic Antibody

Both the T-cell engaging anti-CD3 bispecific antibody, in particular the anti-CEA/anti-CD3 bispecific antibody and the FAP-targeted LTBR agonistic antibody (both called substance herein) or the anti-FolR1/anti-CD3 bispecific antibody and the FAP-targeted LTBR agonistic antibody can be administered by any suitable means, including parenteral, intrapulmonary, and intranasal, and, if desired for local treatment, intralesional administration. The methods disclosed herein are particularly useful, however, in relation to therapeutic agents administered by parenteral, particularly intravenous or subcutaneous infusion, more particularly by subcutaneous infusion..

Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In one embodiment, the therapeutic agent is administered parenterally, particularly intravenously. In a particular embodiment, the therapeutic agent is administered by intravenous infusion.

Both the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Both the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody need not be, but are optionally formulated with one or more agents currently used to prevent or treat the disorder in question. The effective amount of such other agents depends on the amount of therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of disease, the appropriate dosage of the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody (when used in their combination or with one or more other additional therapeutic agents) will depend on the type of disease to be treated, the type of FAP-targeted LTBR agonistic antibody, the severity and course of the disease, whether both agents are administered for preventive or therapeutic purposes, previous therapy, the patient's clinical history and response to the therapeutic agent, and the discretion of the attending physician. Each substance is suitably administered to the patient at one time or over a series of treatments. Depending on the type and severity of the disease, about 1 μg/kg to 15 mg/kg (e.g. 0.1 mg/kg-10 mg/kg) of the substance can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of each substance would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week, every two weeks, or every three weeks (e.g. such that the subject receives from about two to about twenty, or e.g. about six doses of the therapeutic agent). An initial higher loading dose, followed by one or more lower doses, or an initial lower dose, followed by one or more higher doses may be administered. An exemplary dosing regimen comprises administering an initial dose of about 10 mg, followed by a bi-weekly dose of about 20 mg of the therapeutic agent. However, other dosage regimens may be useful. The progress of this therapy is easily monitored by conventional techniques and assays.

In one aspect, the administration of both the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody is a single administration. In certain aspects, the administration of the therapeutic agent is two or more administrations. In one such aspect, the substances are administered every week, every two weeks, or every three weeks, particularly every two or three weeks. In one aspect, the substance is administered in a therapeutically effective amount. In one aspect the substance is administered at a dose of about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg or about 1000 μg/kg. In one aspect, the T-cell engaging anti-CD3 bispecific antibody, in particular the anti-CEA/anti-CD3 bispecific antibody or the anti-FolR1/anti-CD3 bispecific antibody, is administered at a dose which is higher than the dose of the T-cell engaging anti-CD3 bispecific antibody, in particular the anti-CEA/anti-CD3 bispecific antibody or the anti-FolR1/anti-CD3 bispecific antibody, in a corresponding treatment regimen without the administration of the FAP-targeted LTBR agonistic antibody. In one aspect, the administration of the T-cell engaging anti-CD3 bispecific antibody, in particular the anti-CEA/anti-CD3 bispecific antibody or the anti-FolR1/anti-CD3 bispecific antibody, comprises an initial administration of a first dose of the T-cell engaging anti-CD3 bispecific antibody, in particular the anti-CEA/anti-CD3 bispecific antibody or the anti-FolR1/anti-CD3 bispecific antibody, and one or more subsequent administrations of a second dose of the T-cell engaging anti-CD3 bispecific antibody, in particular the anti-CEA/anti-CD3 bispecific antibody or the anti-FolR1/anti-CD3 bispecific antibody, wherein the second dose is higher than the first dose. In one aspect, the administration of the T-cell engaging anti-CD3 bispecific antibody, in particular the anti-CEA/anti-CD3 bispecific antibody or the anti-FolR1/anti-CD3 bispecific antibody, comprises an initial administration of a first dose of the T-cell engaging anti-CD3 bispecific antibody, in particular the anti-CEA/anti-CD3 bispecific antibody or the anti-FolR1/anti-CD3 bispecific antibody, and one or more subsequent administrations of a second dose of the T-cell engaging anti-CD3 bispecific antibody, in particular the anti-CEA/anti-CD3 bispecific antibody or the anti-FolR1/anti-CD3 bispecific antibody, wherein the first dose is not lower than the second dose.

In one aspect, the administration of the T-cell engaging anti-CD3 bispecific antibody, in particular the anti-CEA/anti-CD3 bispecific antibody or the anti-FolR1/anti-CD3 bispecific antibody, in the treatment regimen according to the invention is the first administration of said T-cell engaging anti-CD3 bispecific antibody, in particular the anti-CEA/anti-CD3 bispecific antibody or the anti-FolR1/anti-CD3 bispecific antibody to the subject (at least within the same course of treatment). In one aspect, no administration of the FAP-targeted LTBR agonistic antibody is made to the subject prior to the administration of the T-cell engaging anti-CD3 bispecific antibody, in particular the anti-CEA/anti-CD3 bispecific antibody or the anti-FolR1/anti-CD3 bispecific antibody. In another aspect, the FAP-targeted LTBR agonistic antibody is administered prior to the administration of the T-cell engaging anti-CD3 bispecific antibody, in particular the anti-CEA/anti-CD3 bispecific antibody or the anti-FolR1/anti-CD3 bispecific antibody.

Activation of T cells can lead to severe cytokine release syndrome (CRS). In a phase 1 study conducted by TeGenero (Suntharalingam et al., N Engl J Med (2006) 355, 1018-1028), all 6 healthy volunteers experienced near fatal, severe cytokine release syndrome (CRS) rapidly post-infusion of an inappropriately-dosed, T-cell stimulating super-agonist anti-CD28 monoclonal antibody. The cytokine release associated with administration of a T-cell engaging therapeutic agent, such as the anti-CEA/anti-CD3 bispecific antibody or the anti-FolR1/anti-CD3 bispecific antibody, to a subject can be significantly reduced by pre-treatment of said subject with a Type II anti-CD20 antibody, such as obinutuzumab. The use of GAZYVA® pre-treatment (Gpt) should aid in the rapid depletion of B cells, both in the peripheral blood and in secondary lymphoid organs, such that the risk of highly relevant adverse events (AEs) from strong systemic T cell activation by T-cell engaging therapeutic agents (e.g. CRS) is reduced, while supporting exposure levels of T-cell engaging therapeutic agents that are high enough from the start of dosing to mediate tumour cell elimination. To date, the safety profile of obinutuzumab (including cytokine release) has been assessed and managed in hundreds of patients in ongoing obinutuzumab clinical trials. Finally, in addition to supporting the safety profile of T-cell engaging anti-CD3 bispecific antibodies such as the anti-CEA/anti-CD3 bispecific antibody or the anti-FolR1/anti-CD3 bispecific antibody, Gpt should also help prevent the formation of anti-drug antibodies (ADAs) to these unique molecules. Thus, the T-cell engaging anti-CD3 bispecific antibody, in particular the anti-CEA/anti-CD3 bispecific antibody or the anti-FolR1/anti-CD3 bispecific antibody, is for use in combination with a FAP-targeted LTBR agonistic antibody, wherein a pretreatment with an Type II anti-CD20 antibody, preferably obinutuzumab, is performed prior to the combination treatment.

The combination of the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody can be used in combination with further agents in a therapy. For instance, at least one additional therapeutic agent may be co-administered. In certain aspects, an additional therapeutic agent is an immunotherapeutic agent. In one aspect, the combination of the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody can be used in combination with a PD-1 axis binding antagonist. In one aspect, the PD-1 axis binding antagonist is selected from the group consisting of a PD-1 binding antagonist, a PD-L1 binding antagonist and a PD-L2 binding antagonist. In a particular aspect, PD-1 axis binding antagonist is a PD-1 binding antagonist. In one aspect, the PD-1 axis binding antagonist is selected MDX 1106 (nivolumab), MK-3475 (pembrolizumab), CT-01I (pidilizumab), MEDI-0680 (AMP-514), PDR001, REGN2810, and BGB-108. In another particular aspect, the PD-1 axis binding antagonist is a PD-L1 binding antagonist. In one aspect, the PD-1 axis binding antagonist is selected from MPDL3280A (atezolizumab), YW243.55.S70, MDX-1105, MEDI4736 (durvalumab), and MSB0010718C (avelumab). More particularly, the combination of the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody can be used in combination with MPDL3280A (atezolizumab). In another particular aspect, the combination of the T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody can be used in combination with pembrolizumab.

The period of time between the administration of the PD-1 axis binding antagonist and the administration of the combination therapy comprising T-cell engaging anti-CD3 bispecific antibody, in particular the anti-CEA/anti-CD3 bispecific antibody or the anti-FolR1/anti-CD3 bispecific antibody, and the FAP-targeted LTBR agonistic antibody and the doses are chosen such as to effectively shrink the tumor in the subject prior to administration of the combination therapy.

Such combination therapies noted above encompass combined administration (where two or more therapeutic agents are included in the same or separate formulations), and separate administration, in which case, administration of the therapeutic agent can occur prior to, simultaneously, and/or following, administration of an additional therapeutic agent or agents. In one embodiment, administration of the therapeutic agent and administration of an additional therapeutic agent occur within about one month, or within about one, two or three weeks, or within about one, two, three, four, five, or six days, of each other.

### Therapeutic Methods and Compositions

Bispecific antibodies recognizing two cell surface proteins on different cell populations hold the promise to redirect cytotoxic immune cells for destruction of pathogenic target cells.

In one aspect, provided is a method for treating or delaying progression of cancer in a subject comprising administering to the subject an effective amount of a T-cell engaging anti-CD3 bispecific antibody, in particular a anti-CEA/anti-CD3 bispecific antibody or the anti-FolR1/anti-CD3 bispecific antibody, and a FAP-targeted LTBR agonistic antibody. In one such aspect, the method further comprises administering to the subject an effective amount of at least one additional therapeutic agent. In further embodiments, herein is provided a method for tumor shrinkage comprising administering to the subject an effective amount of a T-cell engaging anti-CD3 bispecific antibody, in particular an anti-CEA/anti-CD3 bispecific antibody or an anti-FolR1/anti-CD3 bispecific antibody, and a FAP-targeted LTBR agonistic antibody. An “individual” or a “subject” according to any of the above aspects is preferably a human.

In further aspects, a composition for use in cancer immunotherapy is provided comprising a T-cell engaging anti-CD3 bispecific antibody, in particular an anti-CEA/anti-CD3 bispecific antibody or an anti-FolR1/anti-CD3 bispecific antibody, and a FAP-targeted LTBR agonistic antibody. In certain aspects, a composition comprising a T-cell engaging anti-CD3 bispecific antibody, in particular an anti-CEA/anti-CD3 bispecific antibody or an anti-FolR1/anti-CD3 bispecific antibody, and a FAP-targeted LTBR agonistic antibody for use in a method of cancer immunotherapy is provided.

In a further aspect, herein is provided for the use of a composition comprising a T-cell engaging anti-CD3 bispecific antibody, in particular an anti-CEA/anti-CD3 bispecific antibody or an anti-FolR1/anti-CD3 bispecific antibody, and a FAP-targeted LTBR agonistic antibody in the manufacture or preparation of a medicament. In one embodiment, the medicament is for treatment of solid tumors. In a further embodiment, the medicament is for use in a method of tumor shrinkage comprising administering to an individual having a solid tumor an effective amount of the medicament. In one such embodiment, the method further comprises administering to the individual an effective amount of at least one additional therapeutic agent. In a further aspect, the medicament is for treating solid tumors.

In certain aspects, the disease to be treated is a proliferative disorder, particularly cancer. Examples of cancers include, but are not limited to, bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other examples of cancer include carcinoma, lymphoma (e.g., Hodgkin's and non-Hodgkin's lymphoma), blastoma, sarcoma, and leukemia. Other cell proliferation disorders that can be treated using the bispecific antigen binding molecule or antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In particular, the cancer is chosen from the group consisting of renal cell cancer, skin cancer, lung cancer, colorectal cancer, breast cancer, brain cancer, head and neck cancer. More particularly, the cancer to be treated by the methods described herein is breast cancer. A skilled artisan readily recognizes that in many cases the bispecific agonistic LTBR antibody may not provide a cure but may provide a benefit. In some aspects, a physiological change having some benefit is also considered therapeutically beneficial. Thus, in some aspects, an amount of the bispecific agonistic LTBR antibody or agonistic LTBR antibody that provides a physiological change is considered an “effective amount” or a “therapeutically effective amount”. In some aspects, the individual has CEA positive cancer. In some aspects, CEA positive cancer is colon cancer, lung cancer, ovarian cancer, gastric cancer, bladder cancer, pancreatic cancer, endometrial cancer, breast cancer, kidney cancer, esophageal cancer, or prostate cancer. In some aspects, the CEA positive cancer is breast cancer. In some aspects, the breast cancer is a breast carcinoma or a breast adenocarcinoma. In some aspects, the breast carcinoma is an invasive ductal carcinoma. In some aspects, the lung cancer is a lung adenocarcinoma. In some aspects, the colon cancer is a colorectal adenocarcinoma. An “individual” according to any of the above embodiments may be a human.

The combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one embodiment, administration of a T-cell engaging anti-CD3 bispecific antibody, in particular an anti-CEA/anti-CD3 bispecific antibody or an anti-FolR1/anti-CD3 bispecific antibody, and a FAP-targeted LTBR agonistic antibody and optionally the administration of an additional therapeutic agent occur within about one month, or within about one, two or three weeks, or within about one, two, three, four, five, or six days, of each other.

Both the T-cell engaging anti-CD3 bispecific antibody, in particular an anti-CEA/anti-CD3 bispecific antibody or an anti-FolR1/anti-CD3 bispecific antibody, and the FAP-targeted LTBR agonistic antibody as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Both the T-cell engaging anti-CD3 bispecific antibody, in particular an anti-CEA/anti-CD3 bispecific antibody or an anti-FolR1/anti-CD3 bispecific antibody, and the FAP-targeted LTBR agonistic antibody as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### Articles of Manufacture (Kits)

In another aspect of the invention, a kit containing materials useful for the treatment, prevention and/or diagnosis of the disorders described above is provided. The kit comprises at least one container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least two active agents in the kit are a T-cell engaging anti-CD3 bispecific antibody, in particular an anti-CEA/anti-CD3 bispecific antibody or an anti-FolR1/anti-CD3 bispecific antibody, and a FAP-targeted LTBR agonistic antibody as disclosed therein.

In a particular aspect, provided is a kit for treating or delaying progression of cancer in a subject, comprising a package comprising (A) a first composition comprising as active ingredient a T-cell engaging anti-CD3 bispecific antibody, in particular an anti-CEA/anti-CD3 bispecific antibody or an anti-FolR1/anti-CD3 bispecific antibody, and a pharmaceutically acceptable carrier; (B) a second composition comprising as active ingredient a FAP-targeted LTBR agonistic antibody and a pharmaceutically acceptable carrier, and (C) instructions for using the compositions in a combination therapy.

The label or package insert indicates how the composition is used for treating the condition of choice and provides the instructions for using the compositions in a combination therapy. Moreover, the kit may comprise (a) a first container with a composition contained therein, wherein the composition comprises a T-cell engaging anti-CD3 bispecific antibody, in particular an anti-CEA/anti-CD3 bispecific antibody or an anti-FolR1/anti-CD3 bispecific antibody, of the invention; and (b) a second container with a composition contained therein, wherein the composition comprises FAP-targeted LTBR agonistic antibody as disclosed herein. In addition, the kit may comprise one or more further containers comprising further active ingredients that can be used in combination. The article of manufacture in this embodiment of the invention may further comprise a package insert indicating that the compositions can be used to treat a particular condition.

Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Name | Sequence |
| 1 | hu LTBR (UniProt Acc. No. | MLLPWATSAPGLAWGPLVLGLFGLLAASQPQAVPP |
| | P36941) | YASENQTCRDQEKEYYEPQHRICCSRCPPGTYVSA |
| | | KCSRIRDTVCATCAENSYNEHWNYLTICQLCRPCD |
| | | PVMGLEEIAPCTSKRKTQCRCQPGMFCAAWALECT |
| | | HCELLSDCPPGTEAELKDEVGKGNNHCVPCKAGHE |
| | | QNTSSPSARCQPHTRCENQGLVEAAPGTAQSDTTC |
| | | KNPLEPLPPEMSGTMLMLAVLLPLAFFLLLATVES |
| | | CIWKSHPSLCRKLGSLLKRRPQGEGPNPVAGSWEP |
| | | PKAHPYFPDLVQPLLPISGDVSPVSTGLPAAPVLE |
| | | AGVPQQQSPLDLTREPQLEPGEQSQVAHGINGIHV |
| | | TGGSMTITGNIYIYNGPVLGGPPGPGDLPATPEPP |
| | | YPIPEEGDPGPPGLSTPHQEDGKAWHLAETEHCGA |
| | | TPSNRGPRNQFITHD |
| 2 | hu FAP (UniProt no. | MKTWVKIVFGVATSAVLALLVMCIVLRPSRVHNSE |
| | Q12884, version 168) | ENTMRALTLKDILNGTFSYKTFFPNWISGQEYLHQ |
| | | SADNNIVLYNIETGQSYTILSNRTMKSVNASNYGL |
| | | SPDRQFVYLESDYSKLWRYSYTATYYIYDLSNGEF |
| | | VRGNELPRPIQYLCWSPVGSKLAYVYQNNIYLKQR |
| | | PGDPPFQITENGRENKIENGIPDWVYEEEMLATKY |
| | | ALWWSPNGKFLAYAEENDTDIPVIAYSYYGDEQYP |
| | | RTINIPYPKAGAKNPVVRIFIIDTTYPAYVGPQEV |
| | | PVPAMIASSDYYFSWLTWVTDERVCLQWLKRVQNV |
| | | SVLSICDFREDWQTWDCPKTQEHIEESRTGWAGGF |
| | | FVSTPVESYDAISYYKIFSDKDGYKHIHYIKDTVE |
| | | NAIQITSGKWEAINIFRVTQDSLFYSSNEFEEYPG |
| | | RRNIYRISIGSYPPSKKCVTCHLRKERCQYYTASF |
| | | SDYAKYYALVCYGPGIPISTLHDGRTDQEIKILEE |
| | | NKELENALKNIQLPKEEIKKLEVDEITLWYKMILP |
| | | PQFDRSKKYPLLIQVYGGPCSQSVRSVFAVNWISY |
| | | LASKEGMVIALVDGRGTAFQGDKLLYAVYRKLGVY |
| | | EVEDQITAVRKFIEMGFIDEKRIAIWGWSYGGYVS |
| | | SLALASGTGLFKCGIAVAPVSSWEYYASVYTEREM |
| | | GLPTKDDNLEHYKNSTVMARAEYFRNVDYLLIHGT |
| | | ADDNVHFQNSAQIAKALVNAQVDFQAMWYSDQNHG |
| | | LSGLSTNHLYTHMTHELKQCESLSD |
| 3 | FAP(4B9) CDR-H1 | SYAMS |
| 4 | FAP(4B9) CDR-H2 | AIIGSGASTYYADSVKG |
| 5 | FAP(4B9) CDR-H3 | GWEGGENY |
| 6 | FAP(4B9) CDR-L1 | RASQSVTSSYLA |
| 7 | FAP(4B9) CDR-L2 | VGSRRAT |
| 8 | FAP(4B9) CDR-L3 | QQGIMLPPT |
| 9 | FAP(4B9) VH | EVQLLESGGGLVQPGGSLRLSCAASGFTESSYAMS |
| | | WVRQAPGKGLEWVSAIIGSGASTYYADSVKGRFTI |
| | | SRDNSKNTLYLQMNSLRAEDTAVYYCAKGWEGGEN |
| | | YWGQGTLVTVSS |
| 10 | FAP(4B9) VL | EIVLTQSPGTLSLSPGERATLSCRASQSVTSSYLA |
| | | WYQQKPGQAPRLLINVGSRRATGIPDRESGSGSGT |
| | | DFTLTISRLEPEDFAVYYCQQGIMLPPTFGQGTKV |
| | | EIK |
| 11 | FAP (28H1) CDR-H1 | SHAMS |
| 12 | FAP (28H1) CDR-H2 | AIWASGEQYYADSVKG |
| 13 | FAP (28H1) CDR-H3 | GWLGNEDY |
| 14 | FAP (28H1) CDR-L1 | RASQSVSRSYLA |
| 15 | FAP (28H1) CDR-L2 | GASTRAT |
| 16 | FAP (28H1) CDR-L3 | QQGQVIPPT |
| 17 | FAP(28H1) VH | EVQLLESGGGLVQPGGSLRLSCAASGFTESSHAMS |
| | | WVRQAPGKGLEWVSAIWASGEQYYADSVKGRFTIS |
| | | RDNSKNTLYLQMNSLRAEDTAVYYCAKGWLGNEDY |
| | | WGQGTLVTVSS |
| 18 | FAP(28H1) VL | EIVLTQSPGTLSLSPGERATLSCRASQSVSRSYLA |
| | | WYQQKPGQAPRLLIIGASTRATGIPDRESGSGSGT |
| | | DFTLTISRLEPEDFAVYYCQQGQVIPPTFGQGTKV |
| | | EIK |
| 19 | LTBR (9459) CDR-H1 | SYTMN |
| 20 | LTBR (9459) CDR-H2 | GVAPSGDRTYYTDSVKG |
| 21 | LTBR (9459) CDR-H3 | GFAFTN |
| 22 | LTBR (9459) CDR-L1 | RASQGISSALA |
| 23 | LTBR (9459) CDR-L2 | DASSLDS |
| 24 | LTBR (9459) CDR-L3 | QQYNNYPLT |
| 25 | LTBR (9459) VH | EVQLVESGGGLVQPGGSLRLSCAASGFTESSYTMN |
| | | WVRQAPGKGLEWVSGVAPSGDRTYYTDSVKGRETI |
| | | SRDNAKNTLDLQMTSLRAEDAAIYICAQGFAFTNW |
| | | GQGTMVTVSS |
| 26 | LTBR (9459) VL | DIQMTQSPSSLSASVGDRVTITCRASQGISSALAW |
| | | FQQKPGKAPKRLIFDASSLDSGVPSRESGSGSGTD |
| | | FTLTISSLQPEDFATYYCQQYNNYPLTFGGGTKVE |
| | | MK |
| 27 | CD3 (CH2527) CDR-H1 | TYAMN |
| 28 | CD3 (CH2527) CDR-H2 | RIRSKYNNYATYYADSVKG |
| 29 | CD3 (CH2527) CDR-H3 | HGNFGNSYVSWFAY |
| 30 | CD3 (CH2527) CDR-L1 | GSSTGAVTTSNYAN |
| 31 | CD3 (CH2527) CDR-L2 | GTNKRAP |
| 32 | CD3 (CH2527) CDR-L3 | ALWYSNLWV |
| 33 | CD3 (CH2527) VH | EVQLLESGGGLVQPGGSLRLSCAASGFTESTYAMN |
| | | WVRQAPGKGLEWVSRIRSKYNNYATYYADSVKGRE |
| | | TISRDDSKNTLYLQMNSLRAEDTAVYYCVRHGNFG |
| | | NSYVSWFAYWGQGTLVTVSS |
| 34 | CD3 (CH2527) VL | QAVVTQEPSLTVSPGGTVTLTCGSSTGAVTTSNYA |
| | | NWVQEKPGQAFRGLIGGTNKRAPGTPARFSGSLLG |
| | | GKAALTLSGAQPEDEAEYYCALWYSNLWVFGGGTK |
| | | LTVL |
| 35 | CD3 (2C11) CDR-H1 | GYGMH |
| 36 | CD3 (2C11) CDR-H2 | YITSSSINIKYADAVKG |
| 37 | CD3 (2C11) CDR-H3 | FDWDKNY |
| 38 | CD3 (2C11) CDR-L1 | QASQDISNYLN |
| 39 | CD3 (2C11) CDR-L2 | YTNKLAD |
| 40 | CD3 (2C11) CDR-L3 | QQYYNYPWT |
| 41 | CD3 (2C11)VH | EVQLVESGGGLVQPGKSLKLSCEASGFTFSGYGMH |
| | | WVRQAPGRGLESVAYITSSSINIKYADAVKGRFTV |
| | | SRDNAKNLLFLQMNILKSEDTAMYYCARFDWDKNY |
| | | WGQGTMVTVSS |
| 42 | CD3 (2C11) VL | DIQMTQSPSSLPASLGDRVTINCQASQDISNYLNW |
| | | YQQKPGKAPKLLIYYTNKLADGVPSRFSGSGSGRD |
| | | SSFTISSLESEDIGSYYCQQYYNYPWTFGPGTKLE |
| | | IK |
| 43 | CEA CDR-H1 | EFGMN |
| 44 | CEA CDR-H2 | WINTKTGEATYVEEFKG |
| 45 | CEA CDR-H3 | WDFAYYVEAMDY |
| 46 | CEA CDR-L1 | KASAAVGTYVA |
| 47 | CEA CDR-L2 | SASYRKR |
| 48 | CEA CDR-L3 | HQYYTYPLFT |
| 49 | CEA VH | QVQLVQSGAEVKKPGASVKVSCKASGYTFTEFGMN |
| | | WVRQAPGQGLEWMGWINTKTGEATYVEEFKGRVTF |
| | | TTDTSTSTAYMELRSLRSDDTAVYYCARWDFAYYV |
| | | EAMDYWGQGTTVTVSS |
| 50 | CEA VL | DIQMTQSPSSLSASVGDRVTITCKASAAVGTYVAW |
| | | YQQKPGKAPKLLIYSASYRKRGVPSRFSGSGSGTD |
| | | FTLTISSLQPEDFATYYCHQYYTYPLFTFGQGTKL |
| | | EIK |
| 51 | P1AG7512(2 + 1) HC knob | DIQMTQSPSSLSASVGDRVTITCRASQGISSALAW |
| | LTBR - FAP | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKENWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGEVQLLESGGGLVQPGGSLRLSCAASGFTFSSYA |
| | | MSWVRQAPGKGLEWVSAIIGSGASTYYADSVKGRF |
| | | TISRDNSKNTLYLQMNSLRAEDTAVYYCAKGWFGG |
| | | FNYWGQGTLVTVSSASTKGPSVFPLAPSSKSTSGG |
| | | TAALGCLVEDYFPEPVTVSWNSGALTSGVHTFPAV |
| | | LQSSGLYSLSSVVTVPSSSLGTQTYICNVNHKPSN |
| | | TKVDEKVEPKSCD |
| 52 | P1AG7512(2 + 1) LC LTBR | EVQLVESGGGLVQPGGSLRLSCAASGFTFSSYTMN |
| | (2x)crossed | WVRQAPGKGLEWVSGVAPSGDRTYYTDSVKGRETI |
| | | SRDNAKNTLDLQMTSLRAEDAAIYICAQGFAFTNW |
| | | GQGTMVTVSSASVAAPSVFIFPPSDEQLKSGTASV |
| | | VCLLNNFYPREAKVQWKVDNALQSGNSQESVTEQD |
| | | SKDSTYSLSSTLTLSKADYEKHKVYACEVTHQGLS |
| | | SPVTKSENRGEC |
| 53 | P1AG7512(2 + 1) HC hole | DIQMTQSPSSLSASVGDRVTITCRASQGISSALAW |
| | LTBR crossed | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKENWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVESCSVM |
| | | HEALHNHYTQKSLSLSPG |
| 54 | P1AG7512(2 + 1) LC FAP | EIVLTQSPGTLSLSPGERATLSCRASQSVTSSYLA |
| | charged | WYQQKPGQAPRLLINVGSRRATGIPDRESGSGSGT |
| | | DETLTISRLEPEDFAVYYCQQGIMLPPTFGQGTKV |
| | | EIKRTVAAPSVFIFPPSDRKLKSGTASVVCLLNNE |
| | | YPREAKVQWKVDNALQSGNSQESVTEQDSKDSTYS |
| | | LSSTLTLSKADYEKHKVYACEVTHQGLSSPVTKSF |
| | | NRGEC |
| 55 | P1AG5459 HC KK + LTBR | EVQLVESGGGLVQPGGSLRLSCAASGFTFSSYTMN |
| | with charges - FAP crossed | WVRQAPGKGLEWVSGVAPSGDRTYYTDSVKGRFTI |
| | (murine 2 + 1 surrogate) | SRDNAKNTLDLQMTSLRAEDAAIYICAQGFAFTNW |
| | | GQGTMVTVSSAKTTPPSVYPLAPGSAAQTNSMVTL |
| | | GCLVEGYFPEPVTVTWNSGSLSSGVHTFPAVLQSD |
| | | LYTLSSSVTVPSSTWPSQTVTCNVAHPASSTKVDE |
| | | KIVPRDCGCKPCICTVPEVSSVFIFPPKPKDVLTI |
| | | TLTPKVTCVVVAISKDDPEVQFSWFVDDVEVHTAQ |
| | | TKPREEQINSTFRSVSELPIMHQDWLNGKEFKCRV |
| | | NSAAFGAPIEKTISKTKGRPKAPQVYTIPPPKKQM |
| | | AKDKVSLTCMITNFFPEDITVEWQWNGQPAENYKN |
| | | TQPIMKTDGSYFVYSKLNVQKSNWEAGNTFTCSVL |
| | | HEGLHNHHTEKSLSHSPGGGGGSGGGGSGGGGSGG |
| | | GGSEVQLLESGGGLVQPGGSLRLSCAASGFTFSSH |
| | | AMSWVRQAPGKGLEWVSAIWASGEQYYADSVKGRF |
| | | TISRDNSKNTLYLQMNSLRAEDTAVYYCAKGWLGN |
| | | FDYWGQGTLVTVSSRADAAPTVSIFPPSSEQLTSG |
| | | GASVVCFLNNFYPKDINVKWKIDGSERQNGVLNSW |
| | | TDQDSKDSTYSMSSTLTLTKDEYERHNSYTCEATH |
| | | KTSTSPIVKSENRNEC |
| 56 | P1AG5459 LC LTBR with | DIQMTQSPSSLSASVGDRVTITCRASQGISSALAW |
| | charges (murine 2 + 1 | FQQKPGKAPKRLIFDASSLDSGVPSRFSGSGSGTD |
| | surrogate) | FTLTISSLQPEDFATYYCQQYNNYPLTFGGGTKVE |
| | | MKRADAAPTVSIFPPSSRKLTSGGASVVCFLNNFY |
| | | PKDINVKWKIDGSERQNGVLNSWTDQDSKDSTYSM |
| | | SSTLTLTKDEYERHNSYTCEATHKTSTSPIVKSEN |
| | | RNEC |
| 57 | P1AG5459 HC DD- LTBR | EVQLVESGGGLVQPGGSLRLSCAASGFTESSYTMN |
| | with charges (murine 2 + 1 | WVRQAPGKGLEWVSGVAPSGDRTYYTDSVKGRFTI |
| | surrogate) | SRDNAKNTLDLQMTSLRAEDAAIYICAQGFAFTNW |
| | | GQGTMVTVSSAKTTPPSVYPLAPGSAAQTNSMVTL |
| | | GCLVEGYFPEPVTVTWNSGSLSSGVHTFPAVLQSD |
| | | LYTLSSSVTVPSSTWPSQTVTCNVAHPASSTKVDE |
| | | KIVPRDCGCKPCICTVPEVSSVFIFPPKPKDVLTI |
| | | TLTPKVTCVVVAISKDDPEVQFSWFVDDVEVHTAQ |
| | | TKPREEQINSTERSVSELPIMHQDWLNGKEFKCRV |
| | | NSAAFGAPIEKTISKTKGRPKAPQVYTIPPPKEQM |
| | | AKDKVSLTCMITNFFPEDITVEWQWNGQPAENYDN |
| | | TQPIMDTDGSYFVYSDLNVQKSNWEAGNTFTCSVL |
| | | HEGLHNHHTEKSLSHSPG |
| 58 | P1AG5459 LC FAP crossed | EIVLTQSPGTLSLSPGERATLSCRASQSVSRSYLA |
| | (murine 2 + 1 surrogate) | WYQQKPGQAPRLLIIGASTRATGIPDRESGSGSGT |
| | | DFTLTISRLEPEDFAVYYCQQGQVIPPTFGQGTKV |
| | | EIKSSAKTTPPSVYPLAPGSAAQTNSMVTLGCLVK |
| | | GYFPEPVTVTWNSGSLSSGVHTFPAVLQSDLYTLS |
| | | SSVTVPSSTWPSQTVTCNVAHPASSTKVDKKIVPR |
| | | DC |
| 59 | Light chain “CEA 2F1” (CEA | DIQMTQSPSSLSASVGDRVTITCKASAAVGTYVAW |
| | TCB) | YQQKPGKAPKLLIYSASYRKRGVPSRFSGSGSGTD |
| | | FTLTISSLQPEDFATYYCHQYYTYPLFTFGQGTKL |
| | | EIKRTVAAPSVFIFPPSDEQLKSGTASVVCLLNNE |
| | | YPREAKVQWKVDNALQSGNSQESVTEQDSKDSTYS |
| | | LSSTLTLSKADYEKHKVYACEVTHQGLSSPVTKSF |
| | | NRGEC |
| 60 | Light Chain humanized CD3 | QAVVTQEPSLTVSPGGTVTLTCGSSTGAVTTSNYA |
| | CH2527 (Crossfab, VL-CH1) | NWVQEKPGQAFRGLIGGTNKRAPGTPARFSGSLLG |
| | (CEA TCB) | GKAALTLSGAQPEDEAEYYCALWYSNLWVFGGGTK |
| | | LTVLSSASTKGPSVFPLAPSSKSTSGGTAALGCLV |
| | | KDYFPEPVTVSWNSGALTSGVHTFPAVLQSSGLYS |
| | | LSSVVTVPSSSLGTQTYICNVNHKPSNTKVDKKVE |
| | | PKSC |
| 61 | CEA_{CHIA1A 98/99} - humanized | QVQLVQSGAEVKKPGASVKVSCKASGYTFTEFGMN |
| | CD3_{CH2527} (Crossfab VH- | WVRQAPGQGLEWMGWINTKTGEATYVEEFKGRVTF |
| | Ck)-Fc(knob) P329GLALA | TTDTSTSTAYMELRSLRSDDTAVYYCARWDFAYYV |
| | (CEA TCB) | EAMDYWGQGTTVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKP |
| | | SNTKVDKKVEPKSCDGGGGSGGGGSEVQLLESGGG |
| | | LVQPGGSLRLSCAASGFTESTYAMNWVRQAPGKGL |
| | | EWVSRIRSKYNNYATYYADSVKGRFTISRDDSKNT |
| | | LYLQMNSLRAEDTAVYYCVRHGNFGNSYVSWFAYW |
| | | GQGTLVTVSSASVAAPSVFIFPPSDEQLKSGTASV |
| | | VCLLNNFYPREAKVQWKVDNALQSGNSQESVTEQD |
| | | SKDSTYSLSSTLTLSKADYEKHKVYACEVTHQGLS |
| | | SPVTKSFNRGECDKTHTCPPCPAPEAAGGPSVELF |
| | | PPKPKDTLMISRTPEVTCVVVDVSHEDPEVKENWY |
| | | VDGVEVHNAKTKPREEQYNSTYRVVSVLTVLHQDW |
| | | LNGKEYKCKVSNKALGAPIEKTISKAKGQPREPQV |
| | | YTLPPCRDELTKNQVSLWCLVKGFYPSDIAVEWES |
| | | NGQPENNYKTTPPVLDSDGSFFLYSKLTVDKSRWQ |
| | | QGNVFSCSVMHEALHNHYTQKSLSLSPG |
| 62 | CEA_{CHIAIA 98/99} (VH-CH1)- | QVQLVQSGAEVKKPGASVKVSCKASGYTFTEFGMN |
| | Fc(hole) P329GLALA (CEA | WVRQAPGQGLEWMGWINTKTGEATYVEEFKGRVTF |
| | TCB) | TTDTSTSTAYMELRSLRSDDTAVYYCARWDFAYYV |
| | | EAMDYWGQGTTVTVSSASTKGPSVFPLAPSSKSTS |
| | | GGTAALGCLVKDYFPEPVTVSWNSGALTSGVHTEP |
| | | AVLQSSGLYSLSSVVTVPSSSLGTQTYICNVNHKP |
| | | SNTKVDKKVEPKSCDKTHTCPPCPAPEAAGGPSVE |
| | | LFPPKPKDTLMISRTPEVTCVVVDVSHEDPEVKEN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSPG |
| 63 | PIAA9604 Light chain “CEA” | DIQMTQSPSSLSASVGDRVTITCKASAAVGTYVAW |
| | (CEA TCB murine surrogate) | YQQKPGKAPKLLIYSASYRKRGVPSRFSGSGSGTD |
| | | FTLTISSLQPEDFATYYCHQYYTYPLFTFGQGTKL |
| | | EIKRADAAPTVSIFPPSSEQLTSGGASVVCELNNF |
| | | YPKDINVKWKIDGSERQNGVLNSWTDQDSKDSTYS |
| | | MSSTLTLTKDEYERHNSYTCEATHKTSTSPIVKSF |
| | | NRNEC |
| 64 | PIAA9604 Light chain “CD3” | DIQMTQSPSSLPASLGDRVTINCQASQDISNYLNW |
| | crossfab (VL-CH1) (CEA TCB | YQQKPGKAPKLLIYYTNKLADGVPSRFSGSGSGRD |
| | murine surrogate) | SSFTISSLESEDIGSYYCQQYYNYPWTFGPGTKLE |
| | | IKSSAKTTPPSVYPLAPGSAAQTNSMVTLGCLVKG |
| | | YFPEPVTVTWNSGSLSSGVHTFPAVLQSDLYTLSS |
| | | SVTVPSSTWPSQTVTCNVAHPASSTKVDKKIVPRD |
| | | C |
| 65 | PIAA9604 CD3-(Crossfab | EVQLVESGGGLVQPGKSLKLSCEASGFTFSGYGMH |
| | VH-Ck)-CEA Fc (DD) | WVRQAPGRGLESVAYITSSSINIKYADAVKGRFTV |
| | DA, PG (CEA TCB murine | SRDNAKNLLFLQMNILKSEDTAMYYCARFDWDKNY |
| | surrogate) | WGQGTMVTVSSASDAAPTVSIFPPSSEQLTSGGAS |
| | | VVCFLNNFYPKDINVKWKIDGSERQNGVLNSWTDQ |
| | | DSKDSTYSMSSTLTLTKDEYERHNSYTCEATHKTS |
| | | TSPIVKSFNRNECGGGGSGGGGSQVQLVQSGAEVK |
| | | KPGASVKVSCKASGYTFTEFGMNWVRQAPGQGLEW |
| | | MGWINTKTGEATYVEEFKGRVTFTTDTSTSTAYME |
| | | LRSLRSDDTAVYYCARWDFAYYVEAMDYWGQGTTV |
| | | TVSSAKTTPPSVYPLAPGSAAQTNSMVTLGCLVKG |
| | | YFPEPVTVTWNSGSLSSGVHTFPAVLQSDLYTLSS |
| | | SVTVPSSTWPSQTVTCNVAHPASSTKVDKKIVPRD |
| | | CGCKPCICTVPEVSSVFIFPPKPKDVLTITLTPKV |
| | | TCVVVAISKDDPEVQFSWFVDDVEVHTAQTKPREE |
| | | QINSTFRSVSELPIMHQDWLNGKEFKCRVNSAAFG |
| | | APIEKTISKTKGRPKAPQVYTIPPPKEQMAKDKVS |
| | | LTCMITNFFPEDITVEWQWNGQPAENYDNTQPIMD |
| | | TDGSYFVYSDLNVQKSNWEAGNTFTCSVLHEGLHN |
| | | HHTEKSLSHSPG |
| 66 | PIAA9604 CEA Fc (KK) | QVQLVQSGAEVKKPGASVKVSCKASGYTFTEFGMN |
| | DA, PG (CEA TCB murine | WVRQAPGQGLEWMGWINTKTGEATYVEEFKGRVTF |
| | surrogate) | TTDTSTSTAYMELRSLRSDDTAVYYCARWDFAYYV |
| | | EAMDYWGQGTTVTVSSAKTTPPSVYPLAPGSAAQT |
| | | NSMVTLGCLVKGYFPEPVTVTWNSGSLSSGVHTFP |
| | | AVLQSDLYTLSSSVTVPSSTWPSQTVTCNVAHPAS |
| | | STKVDKKIVPRDCGCKPCICTVPEVSSVFIFPPKP |
| | | KDVLTITLTPKVTCVVVAISKDDPEVQFSWFVDDV |
| | | EVHTAQTKPREEQINSTERSVSELPIMHQDWLNGK |
| | | EFKCRVNSAAFGAPIEKTISKTKGRPKAPQVYTIP |
| | | PPKKQMAKDKVSLTCMITNFFPEDITVEWQWNGQP |
| | | AENYKNTQPIMKTDGSYFVYSKLNVQKSNWEAGNT |
| | | FTCSVLHEGLHNHHTEKSLSHSPG |
| 67 | monomeric N-terminal | QTCRDQEKEYYEPQHRICCSRCPPGTYVSAKCSRI |
| | human LTbR (ECD full | RDTVCATCAENSYNEHWNYLTICQLCRPCDPVMGL |
| | length) hu IgG1 Fc-fusion | EEIAPCTSKRKTQCRCQPGMFCAAWALECTHCELL |
| | kih HRYF avi | SDCPPGTEAELKDEVGKGNNHCVPCKAGHFQNTSS |
| | (P1AE1217)(knob chain) | PSARCQPHTRCENQGLVEAAPGTAQSDTTCKASGS |
| | | DKTHTCPPCPAPEAAGGPSVELFPPKPKDTLMISR |
| | | TPEVTCVVVDVSHEDPEVKENWYVDGVEVHNAKTK |
| | | PREEQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKTTP |
| | | PVLDSDGSFFLYSKLTVDKSRWQQGNVFSCSVMHE |
| | | ALHNHYTQKSLSLSPGKSGGLNDIFEAQKIEWHE |
| 68 | monomeric N-terminal | DKTHTCPPCPAPELLGGPSVFLFPPKPKDTLMISR |
| | human LTbR (ECD full | TPEVTCVVVDVSHEDPEVKENWYVDGVEVHNAKTK |
| | length) hu IgG1 Fc-fusion | PREEQYNSTYRVVSVLTVLHQDWLNGKEYKCKVSN |
| | kih HRYF avi | KALPAPIEKTISKAKGQPREPQVCTLPPSRDELTK |
| | (P1AE1217)(hole chain) | NQVSLSCAVKGFYPSDIAVEWESNGQPENNYKTTP |
| | | PVLDSDGSFFLVSKLTVDKSRWQQGNVFSCSVMHE |
| | | ALHNRFTQKSLSLSPGK |
| 69 | rbHC.up | AAG CTT GCC ACC ATG GAG ACT GGG CTG |
| | | CGC TGG CTT C |
| 70 | HUJH5.HFc-DO3 | CTT GGG CCC TTG GTA CTT GCT GAG GAG |
| | | ACG GTG ACC AGG G |
| 71 | HUJH6.HFc-Do3 | CTT GGG CCC TTG GTA CTT GCT GAG GAG |
| | | ACG GTG ACC GTG G |
| 72 | BcPCR_FHLC_leader.fw | ACA GAG GAA GCT TGC CGC CAC CAT GGA |
| | (TG) | CAT GAG GGT CCC CGC |
| 73 | HuCK.Do.2AA | TGA AGA CAG ATG GTG CAG CCA CAG |
| 74 | human FAP - Avi - His | RPSRVHNSEENTMRALTLKDILNGTFSYKTFFPNW |
| | biotinylated (P1AA5347) | ISGQEYLHQSADNNIVLYNIETGQSYTILSNRTMK |
| | | SVNASNYGLSPDRQFVYLESDYSKLWRYSYTATYY |
| | | IYDLSNGEFVRGNELPRPIQYLCWSPVGSKLAYVY |
| | | QNNIYLKQRPGDPPFQITENGRENKIENGIPDWVY |
| | | EEEMLATKYALWWSPNGKFLAYAEFNDTDIPVIAY |
| | | SYYGDEQYPRTINIPYPKAGAKNPVVRIFIIDTTY |
| | | PAYVGPQEVPVPAMIASSDYYFSWLTWVTDERVCL |
| | | QWLKRVQNVSVLSICDFREDWQTWDCPKTQEHIEE |
| | | SRTGWAGGFFVSTPVFSYDAISYYKIFSDKDGYKH |
| | | IHYIKDTVENAIQITSGKWEAINIFRVTQDSLFYS |
| | | SNEFEEYPGRRNIYRISIGSYPPSKKCVTCHLRKE |
| | | RCQYYTASFSDYAKYYALVCYGPGIPISTLHDGRT |
| | | DQEIKILEENKELENALKNIQLPKEEIKKLEVDEI |
| | | TLWYKMILPPQFDRSKKYPLLIQVYGGPCSQSVRS |
| | | VFAVNWISYLASKEGMVIALVDGRGTAFQGDKLLY |
| | | AVYRKLGVYEVEDQITAVRKFIEMGFIDEKRIAIW |
| | | GWSYGGYVSSLALASGTGLFKCGIAVAPVSSWEYY |
| | | ASVYTEREMGLPTKDDNLEHYKNSTVMARAEYFRN |
| | | VDYLLIHGTADDNVHFQNSAQIAKALVNAQVDFQA |
| | | MWYSDQNHGLSGLSTNHLYTHMTHFLKQCFSLSVD |
| | | EQLYFQGGSGLNDIFEAQKIEWHEARAHHHHHH |
| 75 | mouse FAP (UniProt | MKTWLKTVFGVTTLAALALVVICIVLRPSRVYKPE |
| | accession no. P97321) | GNTKRALTLKDILNGTFSYKTYFPNWISEQEYLHQ |
| | | SEDDNIVFYNIETRESYIILSNSTMKSVNATDYGL |
| | | SPDRQFVYLESDYSKLWRYSYTATYYIYDLQNGEF |
| | | VRGYELPRPIQYLCWSPVGSKLAYVYQNNIYLKQR |
| | | PGDPPFQITYTGRENRIFNGIPDWVYEEEMLATKY |
| | | ALWWSPDGKFLAYVEENDSDIPIIAYSYYGDGQYP |
| | | RTINIPYPKAGAKNPVVRVFIVDTTYPHHVGPMEV |
| | | PVPEMIASSDYYFSWLTWVSSERVCLQWLKRVQNV |
| | | SVLSICDFREDWHAWECPKNQEHVEESRTGWAGGF |
| | | FVSTPAFSQDATSYYKIFSDKDGYKHIHYIKDTVE |
| | | NAIQITSGKWEAIYIFRVTQDSLFYSSNEFEGYPG |
| | | RRNIYRISIGNSPPSKKCVTCHLRKERCQYYTASF |
| | | SYKAKYYALVCYGPGLPISTLHDGRTDQEIQVLEE |
| | | NKELENSLRNIQLPKVEIKKLKDGGLTFWYKMILP |
| | | PQFDRSKKYPLLIQVYGGPCSQSVKSVFAVNWITY |
| | | LASKEGIVIALVDGRGTAFQGDKFLHAVYRKLGVY |
| | | EVEDQLTAVRKFIEMGFIDEERIAIWGWSYGGYVS |
| | | SLALASGTGLFKCGIAVAPVSSWEYYASIYSEREM |
| | | GLPTKDDNLEHYKNSTVMARAEYFRNVDYLLIHGT |
| | | ADDNVHFQNSAQIAKALVNAQVDFQAMWYSDQNHG |
| | | ISSGRSQNHLYTHMTHFLKQCFSLSD |
| 76 | murine FAP - Avi - His | RPSRVYKPEGNTKRALTLKDILNGTFSYKTYFPNW |
| | biotinylated (P1AD9907) | ISEQEYLHQSEDDNIVFYNIETRESYIILSNSTMK |
| | | SVNATDYGLSPDRQFVYLESDYSKLWRYSYTATYY |
| | | IYDLQNGEFVRGYELPRPIQYLCWSPVGSKLAYVY |
| | | QNNIYLKQRPGDPPFQITYTGRENRIENGIPDWVY |
| | | EEEMLATKYALWWSPDGKFLAYVEENDSDIPIIAY |
| | | SYYGDGQYPRTINIPYPKAGAKNPVVRVFIVDTTY |
| | | PHHVGPMEVPVPEMIASSDYYFSWLTWVSSERVCL |
| | | QWLKRVQNVSVLSICDFREDWHAWECPKNQEHVEE |
| | | SRTGWAGGFFVSTPAFSQDATSYYKIFSDKDGYKH |
| | | IHYIKDTVENAIQITSGKWEAIYIFRVTQDSLFYS |
| | | SNEFEGYPGRRNIYRISIGNSPPSKKCVTCHLRKE |
| | | RCQYYTASFSYKAKYYALVCYGPGLPISTLHDGRT |
| | | DQEIQVLEENKELENSLRNIQLPKVEIKKLKDGGL |
| | | TFWYKMILPPQFDRSKKYPLLIQVYGGPCSQSVKS |
| | | VFAVNWITYLASKEGIVIALVDGRGTAFQGDKELH |
| | | AVYRKLGVYEVEDQLTAVRKFIEMGFIDEERIAIW |
| | | GWSYGGYVSSLALASGTGLFKCGIAVAPVSSWEYY |
| | | ASIYSEREMGLPTKDDNLEHYKNSTVMARAEYERN |
| | | VDYLLIHGTADDNVHFQNSAQIAKALVNAQVDFQA |
| | | MWYSDQNHGILSGRSQNHLYTHMTHFLKQCESLSD |
| | | VDEQLYFQGGSGLNDIFEAQKIEWHEARAHHHHHH |
| 77 | Human CEA (UniProt | MESPSAPPHRWCIPWQRLLLTASLLTEWNPPTTAK |
| | accession no. P06731) | LTIESTPFNVAEGKEVLLLVHNLPQHLFGYSWYKG |
| | | ERVDGNRQIIGYVIGTQQATPGPAYSGREIIYPNA |
| | | SLLIQNIIQNDTGFYTLHVIKSDLVNEEATGQFRV |
| | | YPELPKPSISSNNSKPVEDKDAVAFTCEPETQDAT |
| | | YLWWVNNQSLPVSPRLQLSNGNRTLTLENVTRNDT |
| | | ASYKCETQNPVSARRSDSVILNVLYGPDAPTISPL |
| | | NTSYRSGENLNLSCHAASNPPAQYSWFVNGTFQQS |
| | | TQELFIPNITVNNSGSYTCQAHNSDTGLNRTTVTT |
| | | ITVYAEPPKPFITSNNSNPVEDEDAVALTCEPEIQ |
| | | NTTYLWWVNNQSLPVSPRLQLSNDNRTLTLLSVTR |
| | | NDVGPYECGIQNELSVDHSDPVILNVLYGPDDPTI |
| | | SPSYTYYRPGVNLSLSCHAASNPPAQYSWLIDGNI |
| | | QQHTQELFISNITEKNSGLYTCQANNSASGHSRTT |
| | | VKTITVSAELPKPSISSNNSKPVEDKDAVAFTCEP |
| | | EAQNTTYLWWVNGQSLPVSPRLQLSNGNRTLTLEN |
| | | VTRNDARAYVCGIQNSVSANRSDPVTLDVLYGPDT |
| | | PIISPPDSSYLSGANLNLSCHSASNPSPQYSWRIN |
| | | GIPQQHTQVLFIAKITPNNNGTYACFVSNLATGRN |
| | | NSIVKSITVSASGTSPGLSAGATVGIMIGVLVGVA |
| | | LI |
| 78 | human CD3ϵ (UniProt | MQSGTHWRVLGLCLLSVGVWGQDGNEEMAYKVSIS |
| | accession no. E9PSH8) | GTTVILTCPQYPGSEILWQHNDKNIGGDEDDKNIG |
| | | SDEDHLSLKEFSELEQSGYYVCYPRGSKPEDANFY |
| | | LYLRARVCENCMEMDVMSVATIVIVDICITGGLLL |
| | | LVYYWSKNRKAKAKPVTRGAGAGGRQRGQNKERPP |
| | | PVPNPDYEPIRKGQRDLYSGLNQRRI |
| 79 | cynomolgus CD3ϵ (UniProt | MQSGTRWRVLGLCLLSIGVWGQDGNEEMGSITQTP |
| | accession no. Q95LI5) | YQVSISGTTVILTCSQHLGSEAQWQHNGKNKEDSG |
| | | DRLFLPEFSEMEQSGYYVCYPRGSNPEDASHHLYL |
| | | KARVCENCMEMDVMAVATIVIVDICITLGLLLLVY |
| | | YWSKNRKAKAKPVTRGAGAGGRQRGQNKERPPPVP |
| | | NPDYEPIRKGQQDLYSGLNQRRI |
| 80 | CH1 domain | ASTKGPSVFPLAPSSKSTSGGTAALGCLVKDYFPE |
| | | PVTVSWNSGALTSGVHTFPAVLQSSGLYSLSSVVT |
| | | VPSSSLGTQTYICNVNHKPSNTKVDKKV |
| 81 | CH1 connector C-terminal | EPKSC |
| | end | |
| 82 | Hinge full | DKTHTCPXCP with X being S or P |
| 83 | CH2 domain | APELLGGPSVFLFPPKPKDTLMISRTPEVTCVWDV |
| | | SHEDPEVKENWYVDGVEVHNAKTKPREEQESTYRW |
| | | SVLTVLHQDWLNGKEYKCKVSNKALPAPIEKTISK |
| | | AK |
| 84 | CH3 domain | GQPREPQVYTLPPSRDELTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLSP |
| 85 | Fc IgG1, caucasian allotype | ASTKGPSVFPLAPSSKSTSGGTAALGCLVKDYFPE |
| | | PVTVSWNSGALTSGVHTFPAVLQSSGLYSLSSVVT |
| | | VPSSSLGTQTYICNVNHKPSNTKVDKKVEPKSCDK |
| | | THTCPPCPAPELLGGPSVFLFPPKPKDTLMISRTP |
| | | EVTCVVVDVSHEDPEVKENWYVDGVEVHNAKTKPR |
| | | EEQYNSTYRVVSVLTVLHQDWLNGKEYKCKVSNKA |
| | | LPAPIEKTISKAKGQPREPQVYTLPPSRDELTKNQ |
| | | VSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| 86 | Fc IgG1, afroamerican | ASTKGPSVFPLAPSSKSTSGGTAALGCLVKDYFPE |
| | allotype | PVTVSWNSGALTSGVHTFPAVLQSSGLYSLSSVVT |
| | | VPSSSLGTQTYICNVNHKPSNTKVDKKVEPKSCDK |
| | | THTCPPCPAPELLGGPSVFLFPPKPKDTLMISRTP |
| | | EVTCVVVDVSHEDPEVKENWYVDGVEVHNAKTKPR |
| | | EEQYNSTYRVVSVLTVLHQDWLNGKEYKCKVSNKA |
| | | LPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| 87 | Fc IgG2 | ASTKGPSVFPLAPCSRSTSESTAALGCLVKDYFPE |
| | | PVTVSWNSGALTSGVHTFPAVLQSSGLYSLSSVVT |
| | | VPSSNFGTQTYTCNVDHKPSNTKVDKTVERKCCVE |
| | | CPPCPAPPVAGPSVFLFPPKPKDTLMISRTPEVTC |
| | | VVVDVSHEDPEVQFNWYVDGVEVHNAKTKPREEQF |
| | | NSTFRVVSVLTVVHQDWLNGKEYKCKVSNKGLPAP |
| | | IEKTISKTKGQPREPQVYTLPPSREEMTKNQVSLT |
| | | CLVKGFYPSDISVEWESNGQPENNYKTTPPMLDSD |
| | | GSFFLYSKLTVDKSRWQQGNVFSCSVMHEALHNHY |
| | | TQKSLSLSPGK |
| 88 | Fc IgG3 | ASTKGPSVFPLAPCSRSTSGGTAALGCLVKDYFPE |
| | | PVTVSWNSGALTSGVHTFPAVLQSSGLYSLSSVVT |
| | | VPSSSLGTQTYTCNVNHKPSNTKVDKRVELKTPLG |
| | | DTTHTCPRCPEPKSCDTPPPCPRCPEPKSCDTPPP |
| | | CPRCPEPKSCDTPPPCPRCPAPELLGGPSVELEPP |
| | | KPKDTLMISRTPEVTCVVVDVSHEDPEVQFKWYVD |
| | | GVEVHNAKTKPREEQYNSTFRVVSVLTVLHQDWLN |
| | | GKEYKCKVSNKALPAPIEKTISKTKGQPREPQVYT |
| | | LPPSREEMTKNQVSLTCLVKGFYPSDIAVEWESSG |
| | | QPENNYNTTPPMLDSDGSFFLYSKLTVDKSRWQQG |
| | | NIFSCSVMHEALHNRFTQKSLSLSPGK |
| 89 | Fc IgG4 | ASTKGPSVFPLAPCSRSTSESTAALGCLVKDYFPE |
| | | PVTVSWNSGALTSGVHTFPAVLQSSGLYSLSSVVT |
| | | VPSSSLGTKTYTCNVDHKPSNTKVDKRVESKYGPP |
| | | CPSCPAPEFLGGPSVELFPPKPKDTLMISRTPEVT |
| | | CVVVDVSQEDPEVQFNWYVDGVEVHNAKTKPREEQ |
| | | FNSTYRVVSVLTVLHQDWLNGKEYKCKVSNKGLPS |
| | | SIEKTISKAKGQPREPQVYTLPPSQEEMTKNQVSL |
| | | TCLVKGFYPSDIAVEWESNGQPENNYKTTPPVLDS |
| | | DGSFFLYSRLTVDKSRWQEGNVFSCSVMHEALHNH |
| | | YTQKSLSLSLGK |
| 90 | human FcγRIIIa (Uniprot | MWQLLLPTALLLLVSAGMRTDLPKAVVELEPQWYR |
| | Accession No. P08637) | VLEKDSVTLKCQGAYSPEDNSTQWFHNESLISSQA |
| | | SSYFIDAATVDDSGEYRCQTNLSTLSDPVQLEVHI |
| | | GWLLLQAPRWVEKEEDPIHLRCHSWKNTALHKVTY |
| | | LQNGKGRKYFHHNSDFYIPKATLKDSGSYFCRGLF |
| | | GSKNVSSETVNITITQGLAVSTISSFFPPGYQVSF |
| | | CLVMVLLFAVDTGLYFSVKTNIRSSTRDWKDHKEK |
| | | WRKDPQDK |
| 91 | Mouse LTBR, Mus | MRLPRASSPCGLAWGPLLLGLSGLLVASQPQLVPP |
| | musculus (Uniprot | YRIENQTCWDQDKEYYEPMHDVCCSRCPPGEFVFA |
| | Accession. No. B2RRV3) | VCSRSQDTVCKTCPHNSYNEHWNHLSTCQLCRPCD |
| | | IVLGFEEVAPCTSDRKAECRCQPGMSCVYLDNECV |
| | | HCEEERLVLCQPGTEAEVTDEIMDTDVNCVPCKPG |
| | | HFQNTSSPRARCQPHTRCEIQGLVEAAPGTSYSDT |
| | | ICKNPPEPGAMLLLAILLSLVLFLLFTTVLACAWM |
| | | RHPSLCRKLGTLLKRHPEGEESPPCPAPRADPHFP |
| | | DLAEPLLPMSGDLSPSPAGPPTAPSLEEVVLQQQS |
| | | PLVQARELEAEPGEHGQVAHGANGIHVTGGSVTVT |
| | | GNIYIYNGPVLGGTRGPGDPPAPPEPPYPTPEEGA |
| | | PGPSELSTPYQEDGKAWHLAETETLGCQDL |
| 92 | Peptide linker (G4S) | GGGGS |
| 93 | Peptide linker (G4S)₂ | GGGGSGGGGS |
| 94 | Peptide linker (SG4)₂ | SGGGGSGGGG |
| 95 | Peptide linker G4(SG4)₂ | GGGGSGGGGSGGGG |
| 96 | peptide linker | GSPGSSSSGS |
| 97 | (G4S)₃ peptide linker | GGGGSGGGGSGGGGS |
| 98 | (G4S)₄ peptide linker | GGGGSGGGGSGGGGSGGGGS |
| 99 | peptide linker | GSGSGSGS |
| 100 | peptide linker | GSGSGNGS |
| 101 | peptide linker | GGSGSGSG |
| 102 | peptide linker | GGSGSG |
| 103 | peptide linker | GGSG |
| 104 | peptide linker | GGSGNGSG |
| 105 | peptide linker | GGNGSGSG |
| 106 | peptide linker | GGNGSG |
| 107 | Human Light (74-240) | DGPAGSWEQLIQERRSHEVNPAAHLTGANSSLTGS |
| | | GGPLLWETQLGLAFLRGLSYHDGALVVTKAGYYYI |
| | | YSKVQLGGVGCPLGLASTITHGLYKRTPRYPEELE |
| | | LLVSQQSPCGRATSSSRVWWDSSFLGGVVHLEAGE |
| | | KVVVRVLDERLVRLRDGTRSYFGAFMV |
| 108 | (P035.093) CD3 CDR-H1 | SYAMN |
| 109 | (P035.093) CD3 CDR-H2 | RIRSKYNNYATYYADSVKG |
| 110 | (P035.093) CD3 CDR-H3 | ASNFPASYVSYFAY |
| 111 | (16D5) FolR1 CDR-H1 | NAWMS |
| 112 | (16D5) FolR1 CDR-H2 | RIKSKTDGGTTDYAAPVKG |
| 113 | (16D5) FolRI CDR-H3 | PWEWSWYDY |
| 114 | (P035.093-VL7-46-13) | GSSTGAVTTSNYAN |
| | CDR-L1 | |
| 115 | (P035.093-VL7-46-13) | GTNKRAP |
| | CDR-L2 | |
| 116 | (P035.093-VL7-46-13) | ALWYSNLWV |
| | CDR-L3 | |
| 117 | (CH2527) CD3 VH | EVQLLESGGGLVQPGGSLRLSCAASGFTESSYAMN |
| | | WVRQAPGKGLEWVSRIRSKYNNYATYYADSVKGRF |
| | | TISRDDSKNTLYLQMNSLRAEDTAVYYCVRASNFP |
| | | ASYVSYFAYWGQGTLVTVSS |
| 118 | (16D5) FolR1 VH | EVQLVESGGGLVKPGGSLRLSCAASGFTFSNAWMS |
| | | WVRQAPGKGLEWVGRIKSKTDGGTTDYAAPVKGRF |
| | | TISRDDSKNTLYLQMNSLKTEDTAVYYCTTPWEWS |
| | | WYDYWGQGTLVTVSS |
| 119 | (P035.093-VL7-46-13) VL | QAVVTQEPSLTVSPGGTVTLTCGSSTGAVTTSNYA |
| | | NWVQEKPGQAFRGLIGGTNKRAPGTPARFSGSLLG |
| | | GKAALTLSGAQPEDEAEYYCALWYSNLWVEGGGTK |
| | | LTVL |
| 120 | (16D5)VH-CH1- | EVQLLESGGGLVQPGGSLRLSCAASGFTFSSYAMN |
| | (P035.093) VH-CH1 Fc | WVRQAPGKGLEWVSRIRSKYNNYATYYADSVKGRF |
| | knob PGLALA | TISRDDSKNTLYLQMNSLRAEDTAVYYCVRASNEP |
| | | ASYVSYFAYWGQGTLVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDGGGGSGGGGSEVQLVE |
| | | SGGGLVKPGGSLRLSCAASGFTFSNAWMSWVRQAP |
| | | GKGLEWVGRIKSKTDGGTTDYAAPVKGRFTISRDD |
| | | SKNTLYLQMNSLKTEDTAVYYCTTPWEWSWYDYWG |
| | | QGTLVTVSSASTKGPSVFPLAPSSKSTSGGTAALG |
| | | CLVKDYFPEPVTVSWNSGALTSGVHTFPAVLQSSG |
| | | LYSLSSVVTVPSSSLGTQTYICNVNHKPSNTKVDK |
| | | KVEPKSCDKTHTCPPCPAPEAAGGPSVELFPPKPK |
| | | DTLMISRTPEVTCVVVDVSHEDPEVKENWYVDGVE |
| | | VHNAKTKPREEQYNSTYRVVSVLTVLHQDWLNGKE |
| | | YKCKVSNKALGAPIEKTISKAKGQPREPQVYTLPP |
| | | CRDELTKNQVSLWCLVKGFYPSDIAVEWESNGQPE |
| | | NNYKTTPPVLDSDGSFFLYSKLTVDKSRWQQGNVF |
| | | SCSVMHEALHNHYTQKSLSLSPG |
| 121 | (16D5)VH-CH1-Fc hole | EVQLVESGGGLVKPGGSLRLSCAASGFTESNAWMS |
| | PGLALA | WVRQAPGKGLEWVGRIKSKTDGGTTDYAAPVKGRF |
| | | TISRDDSKNTLYLQMNSLKTEDTAVYYCTTPWEWS |
| | | WYDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVEL |
| | | FPPKPKDTLMISRTPEVTCVVVDVSHEDPEVKENW |
| | | YVDGVEVHNAKTKPREEQYNSTYRVVSVLTVLHQD |
| | | WLNGKEYKCKVSNKALGAPIEKTISKAKGQPREPQ |
| | | VCTLPPSRDELTKNQVSLSCAVKGFYPSDIAVEWE |
| | | SNGQPENNYKTTPPVLDSDGSFFLVSKLTVDKSRW |
| | | QQGNVFSCSVMHEALHNHYTQKSLSLSPG |
| 122 | (P035.093-VL7-46-13) | QAVVTQEPSLTVSPGGTVTLTCGSSTGAVTTSNYA |
| | VL-CL | NWVQEKPGQAFRGLIGGTNKRAPGTPARFSGSLLG |
| | (common light chain) | GKAALTLSGAQPEDEAEYYCALWYSNLWVEGGGTK |
| | | LTVLGQPKAAPSVTLEPPSSEELQANKATLVCLIS |
| | | DFYPGAVTVAWKADSSPVKAGVETTTPSKQSNNKY |
| | | AASSYLSLTPEQWKSHRSYSCQVTHEGSTVEKTVA |
| | | PTECS |
| 123 | Human FolR1 (UniProt no. | MAQRMTTQLLLLLVWVAVVGEAQTRIAWARTELLN |
| | P15328) | VCMNAKHHKEKPGPEDKLHEQCRPWRKNACCSTNT |
| | | SQEAHKDVSYLYRENWNHCGEMAPACKRHFIQDTC |
| | | LYECSPNLGPWIQQVDQSWRKERVLNVPLCKEDCE |
| | | QWWEDCRTSYTCKSNWHKGWNWTSGENKCAVGAAC |
| | | QPFHFYFPTPTVLCNEIWTHSYKVSNYSRGSGRCI |
| | | QMWFDPAQGNPNEEVARFYAAAMSGAGPWAAWPEL |
| | | LSLALMLLWLLS |
| 124 | Murine FolR1 (UniProt no. | MAHLMTVQLLLLVMWMAECAQSRATRARTELLNVC |
| | P35846) | MDAKHHKEKPGPEDNLHDQCSPWKTNSCCSTNTSQ |
| | | EAHKDISYLYRFNWNHCGTMTSECKRHFIQDTCLY |
| | | ECSPNLGPWIQQVDQSWRKERILDVPLCKEDCQQW |
| | | WEDCQSSFTCKSNWHKGWNWSSGHNECPVGASCHP |
| | | FTFYFPTSAALCEEIWSHSYKLSNYSRGSGRCIQM |
| | | WEDPAQGNPNEEVARFYAEAMSGAGFHGTWPLLCS |
| | | LSLVLLWVIS |
| 125 | Cynomolgus FolR1 (UniProt | MAQRMTTQLLLLLVWVAVVGEAQTRTARARTELLN |
| | no. G7PR14) | VCMNAKHHKEKPGPEDKLHEQCRPWKKNACCSTNT |
| | | SQEAHKDVSYLYRENWNHCGEMAPACKRHFIQDTC |
| | | LYECSPNLGPWIQQVDQSWRKERVLNVPLCKEDCE |
| | | RWWEDCRTSYTCKSNWHKGWNWTSGENKCPVGAAC |
| | | QPFHFYFPTPTVLCNEIWTYSYKVSNYSRGSGRCI |
| | | QMWFDPAQGNPNEEVARFYAAAMSGAGPWAAWPLL |
| | | LSLALTLLWLLS |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, MD (1991)) as defined above.

### EXAMPLES

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

### Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

### Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Preparation, Purification and Characterization of Anti-FAP/Anti-LTBR Bispecific Antibodies

### 1.1 LTBR Antibody Generation by Rabbit Immunization

For immunization, CD rats obtained from Charles River Laboratories International, Inc. as well as Roche proprietary transgenic rabbits comprising a human immunoglobulin locus as reported in WO 2000/46251, WO 2002/12437, WO 2005/007696, WO 2006/047367, US 2007/0033661, and WO 2008/027986 were used. The animals were housed according to the Appendix A “Guidelines for accommodation and care of animals” in an AAALAC-accredited animal facility. All animal immunization protocols and experiments were approved by the Government of Upper Bavaria (permit number 55.2-1-54-2531-66-16 and 55.2-1-54-2532-90-14) and performed according to the German Animal Welfare Act and the Directive 2010/63 of the European Parliament and Council.

Recombinant monomeric N-terminal human LTBR (ECD full length) hu IgG1 Fc-fusion kih HRYF avi (P1AE1217) was used for the immunization of 3 transgenic rabbits. Each rabbit was initially immunized intradermally with 400 μg of the protein, followed by alternating intramuscular and subcutaneous injections of 200 μg of the protein at days 7, 14, 42, 70 and 98. A mixture of TLR agonists was used as adjuvant for each immunization. Blood samples were taken after the third, fourth, fifth and sixth immunization (at 5-7 days post immunization) and used as a source of antigen-specific B-cells. Antigen-specific titers were monitored during the immunization period by ELISA analysis of serum samples.

CD rats (n=4) were immunized with the same immunogen, i.e. recombinant monomeric N-terminal human LTBR (ECD full length) hu IgG1 Fc-fusion kih HRYF avi (P1AE1217) comprising the knob and hole chain of SEQ ID NO:67 and SEQ ID NO:68, respectively. For the initial immunization, 40 μg of the immunogen was emulsified with Incomplete Freund's adjuvant (IFA) and a TLR agonist and one half of the mixture was injected subcutaneously (distributed over several injection sites), while the other half was administered intraperitoneally. After six weeks, a booster immunization was given in the same manner except for the use of IFA, which was substituted by PBS. Four days after the second immunization, blood was drawn and used as a source of antigen-specific B-cells.

### 1.1.1 Isolation of Peripheral Blood Mononuclear Cells (PBMCs)

EDTA containing whole blood was diluted two-fold with 1×PBS (Pan Biotech, Aidenbach, Germany) before density centrifugation using lymphocyte mammal (Cedarlane Laboratories, Burlington, Ontario, Canada) according to the specifications of the manufacturer. The PBMCs were washed twice with 1×PBS.

### 1.1.2 Rabbit B-Cell Cloning Procedure

Depletion of cells: Sterile 6-well plates (cell culture grade) covered with a confluent monolayer of CHO cells or uncovered, were used to deplete non-specifically binding lymphocytes as well as macrophages/monocytes through unspecific adhesion to the plastic. Each well was filled at maximum with 4 mL medium and up to 6×10⁶ PBMCs and allowed to bind for 1 h at 37° C. in the incubator. The cells in the supernatant (peripheral blood lymphocytes (PBLs)) were used for the antigen panning step.

Enrichment of B-cells on human and cynomolgus LTBR: 6-well tissue culture plates covered with a monolayer of human or cynomolgus LTBR-positive CHO cells or coated with recombinant human LTBR-Fc fusion protein were seeded with up to 6×10⁶ PBLs per 4 mL medium and allowed to bind for 1 h at 37° C. in the incubator. Non-adherent cells were removed by carefully washing the wells 1-2 times with 1×PBS. The remaining sticky cells were detached by trypsin for 10 min. at 37° C. in the incubator. Trypsination was stopped with EL-4 B5 medium. The cells were kept on ice until the immune fluorescence staining.

Immune fluorescence staining and flow cytometry: The anti-IgG FITC (Abcam, Cambridge, UK) was used for single cell sorting. For surface staining, cells from the depletion and enrichment step were incubated with the anti-IgG FITC antibody in PBS and incubated for 45 min. in the dark at 4° C. After staining, the PBMCs were washed two-fold with ice-cold PBS. Finally, the PBMCs were resuspended in ice-cold PBS and immediately subjected to the FACS analyses. Propidium iodide in a concentration of 5 μg/mL (BD Pharmingen, San Diego, CA, USA) was added prior to the FACS analyses to discriminate between dead and live cells. A Becton Dickinson FACSAria equipped with a computer and the FACSDiva software (BD Biosciences, USA) was used for single cell sort.

B-cell cultivation: The cultivation of the B-cells was prepared by a method similar to that described by Lightwood et al. (J Immunol Methods, 2006, 316: 133-143). Briefly, single sorted rabbit B-cells were incubated in 96-well plates with 200 μL/well EL-4 B5 medium containing Pansorbin Cells (1:100000) (Calbiochem (Merck), Darmstadt, Deutschland), a cytokine mix (Miltenyi, Bergisch Gladbach, Germany) combined with PMA (Sigma, Darmstadt, Germany) according to WO/2018/122147 and gamma-irradiated murine EL-4-B5 thymoma cells (5×10⁴/well) for 7 days at 37° C. in an atmosphere of 5% CO₂ in the incubator. The supernatants of the B-cell cultivation were removed for screening and the remaining cells were harvested immediately and were frozen at −80° C. in 100 μL RLT buffer (Qiagen, Hilden, Germany).

EL-4 B5 medium consists of RPMI 1640 (Pan Biotech, Aidenbach, Germany) supplemented with 10% FCS, 10 mM HEPES (PAN Biotech, Aidenbach, Germany), 2 mM glutamine, 1% penicillin/streptomycin solution (PAA, Pasching, Austria), 2 mM sodium pyruvate and 0.05 mM β-mercaptoethanol (Gibco, Paisley, Scotland).

PCR amplification of rabbit V-domains: Total RNA was prepared from B-cells lysate (resuspended in RLT buffer—Qiagen—Cat. No 79216) using the NucleoSpin 8/96 RNA kit (Macherey&Nagel; 740709.4, 740698) according to manufacturer's protocol. RNA was eluted with 60 μL RNAse free water. 6 μL of RNA was used to generate cDNA by reverse transcriptase reaction using the Superscript III First-Strand Synthesis SuperMix (Invitrogen 18080-400) and an oligo-dT-primer according to the manufacturer's instructions. All steps were performed on a Hamilton ML Star System. 4 μL of cDNA were used to amplify the immunoglobulin heavy and light chain variable regions (VH and VL) with the AccuPrime Supermix (Invitrogen 12344-040) in a final volume of 50 μL using the primers rbHC.up (SEQ ID NO:69), HUJH5.HFc-D03 (SEQ ID NO:70) and HUJH6.HFc-DO3 (SEQ ID NO:71) for the heavy chain and BcPCR_FHLC_leader.fw (TG) (SEQ ID NO:72) and HuCK.Do.2AA (SEQ ID NO:73) for the light chain of transgenic rabbit B-cells. All forward primers were specific for the signal peptide (of VH and VL, respectively) whereas the reverse primers were specific for the framework or constant regions (of VH and VL, respectively). The PCR conditions for the VH and VL were as follows: Hot start at 94° C. for 5 min.; 35 cycles of 20 sec. at 94° C., 20 sec. at 70° C., 45 sec. at 68° C., and a final extension at 68° C. for 7 min. 8 μL of 50 μL PCR solution were loaded on a 48 E-Gel 2% (Invitrogen G8008-02). Positive PCR reactions were cleaned using the NucleoSpin Extract II kit (Macherey&Nagel; 740609250) according to manufacturer's protocol and eluted with 75 μL elution buffer. All cleaning steps were performed on a Hamilton ML Starlet System.

### 1.2 Expression of Monoclonal LTBR Antibodies

Generation of recombinant vectors for the expression of monoclonal antibodies: For recombinant expression of rabbit monoclonal bivalent antibodies, PCR-products coding for VH or VL were cloned as cDNA into expression vectors by the overhang cloning method (RS Haun et al., Biotechniques (1992) 13, 515-518; M Z Li et al., Nature Methods (2007) 4, 251-256). The expression vectors contained an expression cassette consisting of a 5′ CMV promoter including Intron A, and a 3′ BGH polyadenylation sequence. In addition to the expression cassette, the plasmids contained a pUC18-derived origin of replication and a beta-lactamase gene conferring ampicillin resistance for plasmid amplification in E. coli. Three variants of the basic plasmid were used: One plasmid containing the rabbit IgG constant region designed to accept the VH region from DNA immunized rabbits, one plasmid containing the human IgG constant region with a PG LALA mutation designed to accept the VH regions from protein immunized rabbits and one plasmid containing human kappa LC constant region to accept the VL regions.

Linearized expression plasmids coding for the kappa or gamma constant region and VL/VH inserts were amplified by PCR using overlapping primers. Purified PCR products were incubated with T4 DNA-polymerase which generated single-strand overhangs. The reaction was stopped by dCTP addition. In a next step, plasmid and insert were combined and incubated with recA which induced site specific recombination. The recombined plasmids were transformed into E coli. The next day, the grown colonies were picked and tested for correct recombined plasmid by plasmid preparation and DNA-sequencing. In contrast to the plasmids encoding the rabbit antibody sequences, the genes for the rat antibody sequences have been synthesized at TWIST Bioscience.

For antibody expression, the isolated HC and LC plasmids were transiently co-transfected into HEK293 cells and the supernatants were harvested after 1 week.

Transient expression of IgGs: The antibodies were produced in transiently transfected Expi293F cells (human embryonic kidney cell line 293-derived) cultivated in Expi293 Medium (Invitrogen Corp.). For transfection, lipid-based ExpiFectamine 293 transfection Reagent (Invitrogen Corp) was used. Antibodies were expressed from individual expression plasmids for the IgG light and heavy chains. Transfections were performed as specified in the manufacturer's instructions. Recombinant protein-containing cell culture supernatants were harvested six days after transfection. Supernatants were stored at reduced temperature (e.g. −80° C.) until purification. General information regarding the recombinant expression of human immunoglobulins in e.g. HEK293 cells is given in: Meissner, P. et al., Biotechnol. Bioeng. 75 (2001) 197-203. A particular IgGs resulting from rabbit immunization that was selected for the bispecific antibodies described herein was P1AE9459 with aVH comprising the amino acid sequence of SEQ ID NO:25 and VL comprising the amino acid sequence of SEQ ID NO:26.

### 1.3 Generation and Production of Anti-FAP/Anti-LTBR Bispecific Antibodies

After the generation and characterization of the monospecific anti-LTBR IgGs preferred agonistic anti-LTBR IgGs were converted into 1+1 (monovalent for LTBR) and 2+1 (bivalent for LTBR) FAP-targeting bispecific antibodies. The generation and preparation of anti-FAP clones 28H1 and 4B9 is disclosed in WO 2012/020006 A2, which is incorporated herein by reference.

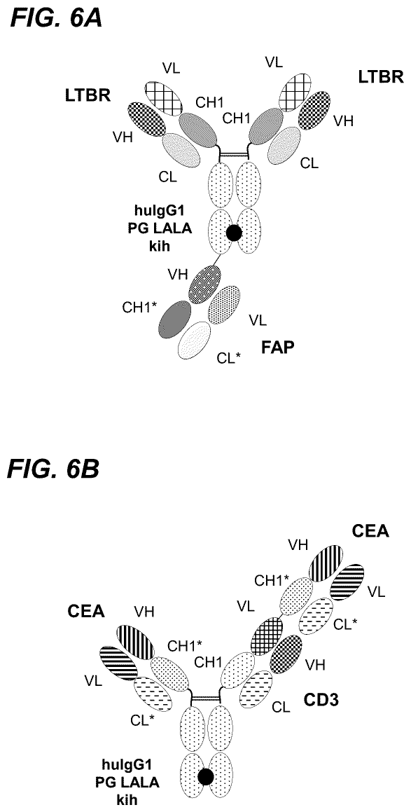

The bispecific anti-FAP/anti-LTBR antibodies were constructed as Fc knobs-into-holes IgGs with CrossMab technology. This means that for the generation of unsymmetric bispecific antibodies, Fc domain subunits contained either the “knob” or “hole” mutations to avoid mispairing of the heavy chains. In order to avoid mispairing of light chains in bispecific antibodies, exchange of VH/VL or CH1/Ckappa domains was introduced in one binding moiety (CrossFab technology). For certain antibody constructs, the VH and VL domains of the Fab arm with LTBR-specificity were crossed and the CH1 and Ckappa domains of the Fab arm with FAP-specificity were equipped with complementary charges, two negatively charged glutamates in CH1 and a positively charged arginine and lysine in Ckappa. In other constructs, the CH1 and Ckappa domains of the Fab arm with FAP-specificity were crossed. Pro329Gly, Leu234Ala and Leu235Ala mutations (PG-LALA) were introduced in the constant region of the human IgG1 heavy chains to abrogate binding to Fc gamma receptors (see FIG. 6A). For murine surrogate antibodies, V-domains were kept human, all constant antibody domains were murine. The murine IgG1 Fc was heterodimerized by complementary charges in CH3 (KK+ and DD− chains) and silencing of the Fc's effector functions was achieved by the introduction of DA PG mutations in CH2.

The bispecific anti-FAP/anti-LTBR antibodies were expressed in Expi293 (HEK) cells. All bispecific anti-FAP/anti-LTBR antibodies were purified using a combined protein A affinity chromatography (Protein A (MabSelect™ SuRe™, Cytiva) and cation exchange chromatography (POROS™ XS) method followed by a preparative size-exclusion chromatography (HiLoad® 16/600 Superdex® S200, Cytiva). Purity of the purified bispecific antibodies was determined by analytical size-exclusion-chromatography (e.g. TSK G3000 SWXL) and CE-SDS (e.g. Caliper LabChip GXII). The identities of the bispecific antibodies were confirmed by LC-MS detecting the theoretical masses of the reduced and non-reduced antibodies. Endotoxin levels have been determined and were all below 0.33 EU/mg.

TABLE 1
FAP-LTBR bispecific antibodies described herein

| | | composed of |
|---|---|---|
| P1AG7512 | 2 + 1 bispecific antibody | SEQ ID NO: 51, |
| | comprising LTBR antibody | SEQ ID NO: 52 (2 times), |
| | P1AE9459 and FAP (4B9) | SEQ ID NO: 53 and |
| | | SEQ ID NO: 54 |
| P1AG5459 | 2 + 1 bispecific antibody | SEQ ID NO: 55, |
| | comprising LTBR antibody | SEQ ID NO: 56 (2 times), |
| | P1AE9459 and FAP (28H1) | SEQ ID NO: 57 and |
| | | SEQ ID NO: 58 |

### Example 2

Preparation, Purification and Characterization of T-Cell Bispecific (TCB) Antibodies TCB molecules have been prepared according to the methods described in WO 2014/131712 A1 or in WO 2016/079076 A1.

The preparation of the anti-CEA/anti-CD3 bispecific antibody (CEA CD3 TCB or CEA TCB) used in the experiments is described in Example 3 of WO 2014/131712 A1. CEA CD3 TCB is a “2+1 IgG CrossFab” antibody and is comprised of two different heavy chains and two different light chains (see FIG. 6B). Point mutations in the CH3 domain (“knobs into holes”) were introduced to promote the assembly of the two different heavy chains. Exchange of the VH and VL domains in the CD3 binding Fab were made in order to promote the correct assembly of the two different light chains. 2+1 means that the molecule has two antigen binding domains specific for CEA and one antigen binding domain specific for CD3.

CEA CD3 TCB comprises the amino acid sequences of SEQ ID NO:59 (two times), SEQ ID NO:60, SEQ ID NO:61 and SEQ ID NO:62.

A murine surrogate molecule P1AA9604 wherein the IgG1 Fc was heterodimerized by complementary charges in CH3 (KK+ and DD− chains) and silencing of the Fc's effector functions was achieved by the introduction of DA PG mutations in CH2, was also prepared. It comprises the amino acid sequences of SEQ ID NO:63 (two times), SEQ ID NO:64, SEQ ID NO:65 and SEQ ID NO:66.

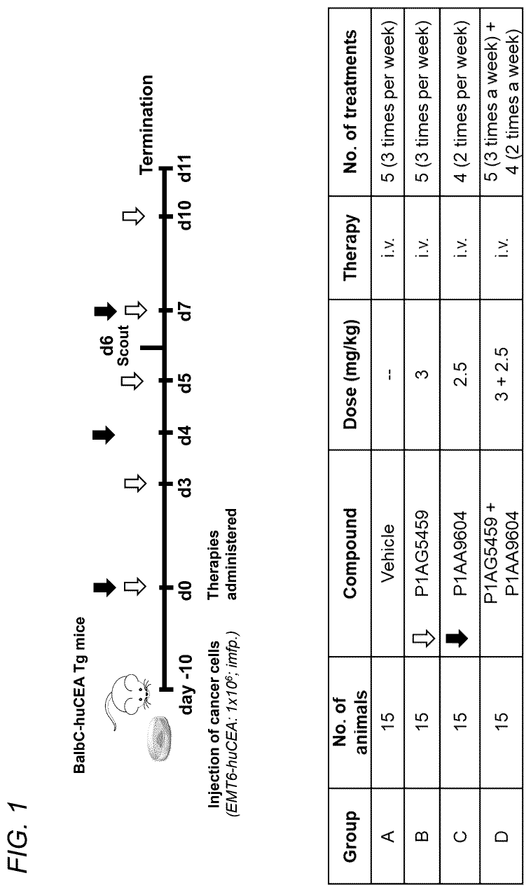

The preparation of the anti-FolR1/anti-CD3 bispecific antibody (FolR1 CD3 TCB or FolR1 TCB) used in the experiments is described in WO 2016/079076 A1. FolR1 CD3 TCB (P1AK1120) is shown as “FolR1 TCB 2+1 classical (common light chain)” in FIG. 1D of WO 2016/079076 and is comprised of two different heavy chains and three times the same VLCL light chain (common light chain). Point mutations in the CH3 domain (“knobs into holes”) were introduced to promote the assembly of the two different heavy chains. 2+1 means that the molecule has two antigen binding domains specific for FolR1 and one antigen binding domain specific for CD3. The CD3 binder is fused at the C-terminus of the Fab heavy chain to the N-terminus of the first subunit of the Fc domain comprising the knob mutation.

P1AK1120 (FolR1 CD3 TCB) comprises a first heavy chain comprising the amino acid sequence of SEQ ID NO:120, a second heavy chain comprising the amino acid sequence of SEQ ID NO:121 and three times a common light chain of SEQ ID NO: 122.

### Example 3

In Vivo Efficacy and Pharmacodynamics Study with FAP-LTBR Surrogate Molecule in Combination with CEA TCB in an Orthotopic Breast Cancer Model

The anti-tumor efficacy and pharmacodynamics effect on immune infiltration, HEV differentiation and cytokine secretion of P1AG5459, a mouse surrogate anti-FAP/anti-LTBR bispecific antibody, in monotherapy or in combination with P1AA9604, a human CEA-murine CD3 T cell bispecific antibody surrogate, was assessed in mice bearing orthotopic breast tumors (EMT6) expressing the human carcinoembryonic antigen (human CEA) and implanted in human CEA transgenic mice, tolerant to the human CEA antigen.

EMT6 cells were obtained from ATCC (CRL-2755) and engineered in-house to express the human carcinoembryonic antigen (EMT6-huCEA). Cells were cultured in DMEM+15% fetal calf serum (FCS). Before injecting, cells were counted and 1,000,000 cells were injected in a total volume of 50 μl, in a 1:1 mix with RPMI and Matrigel, into the mammary fat pad of BALB/c-huCEA mice. These mice were obtained by backcrossing C57BL6-huCEA mice (Clarke et al; Cancer Res. 1998, 58(7), 1469-1477) to the Balb/c genetic background. Tumor growth was measured at least twice weekly using a caliper and tumor volume was calculated as follows: Tumor volume=(W²/2)×L (W: Width, L: Length).

Therapy was started 10 days after tumor cell inoculation at an average tumor volume of 190-200 mm³ per group. P1AG5459 was injected at 3 mg/kg intravenously three times a week, and P1AA9604 was injected at 2.5 mg/kg intravenously two times a week (see treatment scheme in FIG. 1). Tumor growth data expressed as % change from baseline (e.i. tumor volume at randomization date) were plotted in GraphPad PRISM 8 and in a proprietary R-script (Waterfall plot). Time-to-event/Survival data were plotted as Kaplan-Meier plots in a proprietary R-script. In this study an “event” was defined as reaching a tumor size of 290 mm³ which was calculated to be critical tumor burden by: CTB=round(max(TV[baseline])+SD(TV[baseline])/10)*10. For the calculation of the time until the critical tumor burden is reached, a linear model of the following form was fitted for each animal: log(TV)=Day+Day 2 where Day+Day 2 represents the tumor growth kinetics over time. A prediction of the time point at which the critical tumor burden was reached is then made based on the respective model for each animal. Statistical analysis of the Kaplan-Meier plot was performed by Log-Rank test with p-values corrected for multiple testing using the Bonferroni-Holm method in R.

Serum and tumors were collected 6 days after therapy started. Tumor lysates were assessed using a BCA kit (Thermo Scientific, Pierce BCA Protein Assay Kit, #23225, US) according to manufacturer's instructions and the plate was read on the Spectramax i3 ELISA reader (Molecular Devices, US). Then the Bio-Plex Pro™ Mouse Chemokine Panel 33-Plex (#12002231, BioRad, US) was used for cytokine and chemokine level measurement in the protein lysate and serum. The samples were prepared according to the manufacturer's instructions in a 96-well mag-plates (Bio-Plex Pro™ Flat Bottom Plates, #171025001, BioRad, US). The assay was read on the Flexmap 3D® machine (Luminex, US) following the instructions of the xPonent® software, version 4.2, provided with the machine. Data were plotted in GraphPad PRISM 8 and statistical analysis was performed by One-Way Anova tests with subsequent multiple comparison testing with Tukey correction.

The pharmacodynamic effects on immune cell infiltration and HEV differentiation were assessed by histological analysis. Tumor harvest was carried out 6 days after therapy started. Tumors were fixed in 10% formalin (Sigma) and later processed for FFPET (Sakura, VIP6). Three-micrometer paraffin sections were subsequently cut in a microtome (Leica, RM2235). Immunohistochemistry was performed in paraffin sections with the indicated anti-mouse antibodies: B220 (BD Bioscience, #BD553084); PNAd (BioLegend, #120801); muCD8a (eBioscience, #14-0808-82). Staining was performed in the Leica autostainer (Leica, ST5010 and Bond III or RX) following the manufacturer's protocols and scanning was performed on the Olympus VS200 Virtual Slide Microscope scanner. Cell quantification was carried out in Halo software (Indica Labs) and plotting and statistical calculations were carried out in GraphPad Prism by One-way Anova test with subsequent multiple comparison testing with Tukey correction.

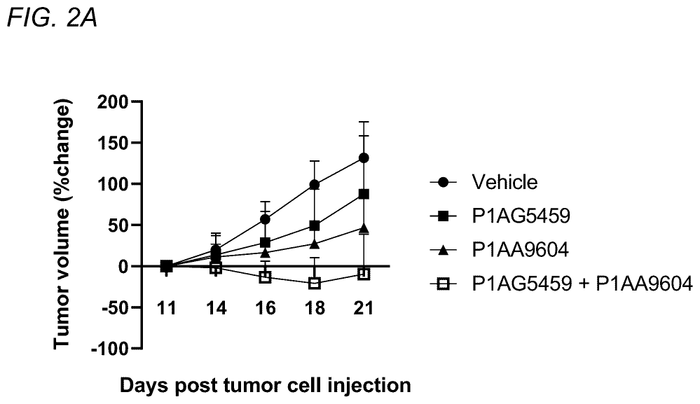

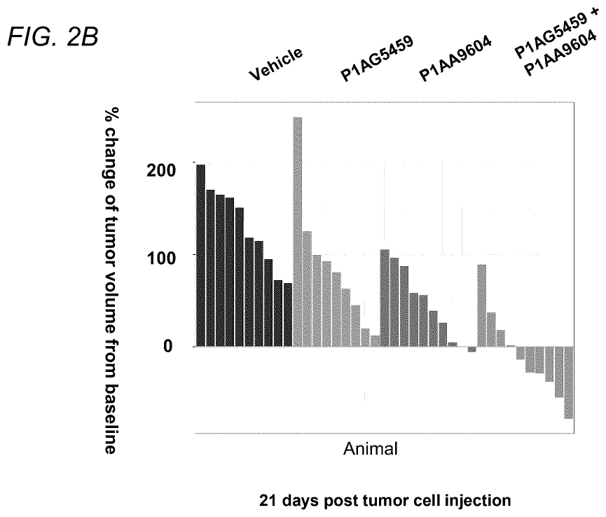

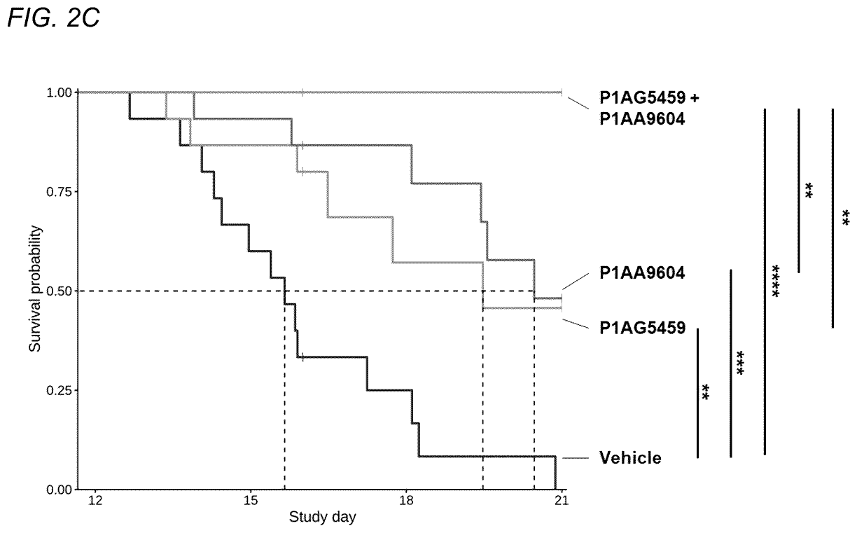

In this study (FIG. 1) we observed that combination treatment with P1AA9604 and P1AG5459 effectively controlled EMT6-huCEA tumor growth (FIGS. 2A-C). At the end of the study, 21 days after tumor cell inoculation, 6 out of 10 mice (60%) in the combination treatment group experienced tumor regression, whereas only 1 out of 10 mice (10%) in the P1AA9604 monotherapy arm, and none in the P1AG5459 monotherapy arm, showed tumor regression (FIG. 2B). Survival/Time to event analysis showed that treatment with P1AG5459 or P1AA9604 as monotherapy significantly improved survival/time to event as compared to the vehicle control arm (p=0.0065 and p=0.0007, respectively). However, the combination of P1AG5459 and P1AA9604 was significantly better in improving survival/time to event as compared to either monotherapy alone (FIG. 2C; p=0.0065 combination vs P1AG5459, p=0.0076 combination vs P1AA9604).

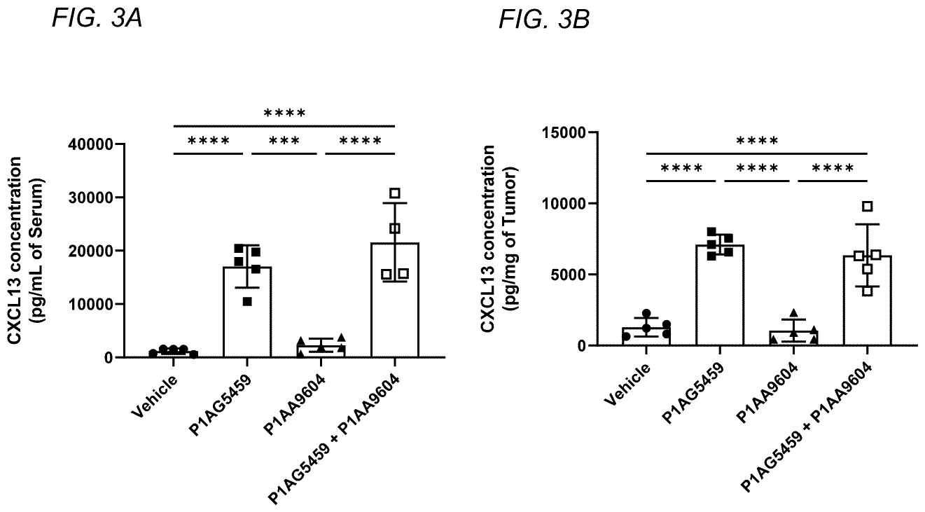

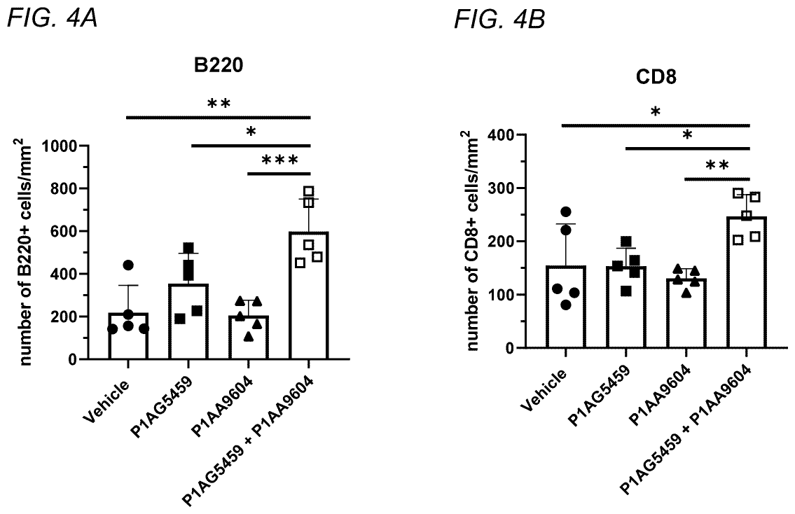

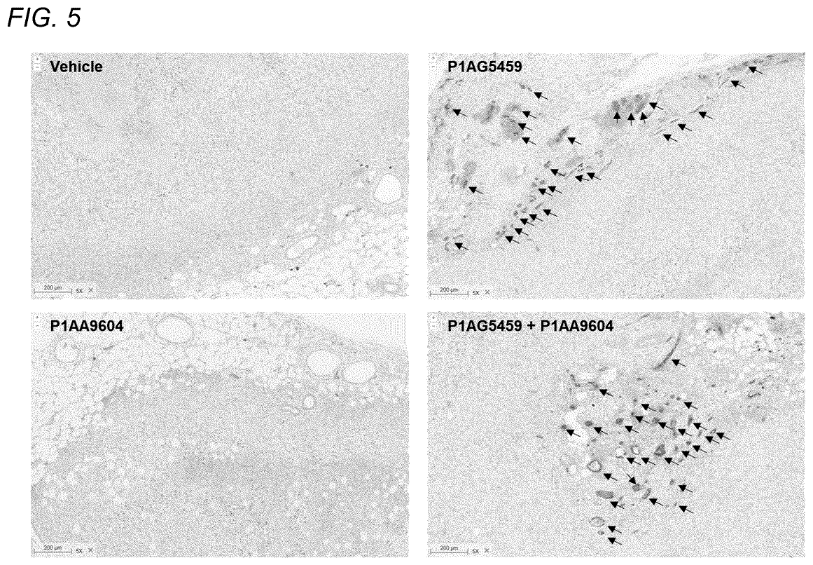

Cytokine analysis showed that treatment with P1AG5459 induced a significant increase in the levels of CXCL13 in both serum and tumors (FIG. 3A-B). Importantly, no such increase was induced by treatment with P1AA9604, suggesting that measuring CXCL13 levels in the serum and/or tumors could serve as a FAP-LTBR specific biomarker of drug activity. Chemokine upregulation at the tumor site in response to FAP-LTBR treatment can mediate the attraction of more immune cells at the tumor site. Indeed, histological analysis revealed that treatment with the combination of P1AA9604 and P1AG5459 increased the infiltration of B cells (FIG. 4A) and CD8+ T cells (FIG. 4B) into the tumor. Moreover, blood vessels expressing the HEV marker pNAD, which are described to be major sites of lymphocyte entry into tumors, could be detected in tumors treated with P1AG5459, alone or in combination with P1AA9604 (FIG. 5, black arrows). Importantly, HEV are not found in untreated tumors, or in tumors treated with P1AA9604 monotherapy, suggesting that HEV differentiation is a specific effect induced by LTBR agonism.

Taken together, the data described in this example show that the combination of an anti-FAP/anti-LTBR bispecific antibody with a T cell engager is efficacious in vivo and superior to either monotherapy in controlling growth of an orthotopic breast cancer model and improving survival, suggesting that this combination can be utilized to maximize responses to T cell engagers. The data also show that the combination therapy modulates the tumor microenvironment and induces the differentiation of vessels to HEVs, the secretion of chemoattractants (like CXCL13) and consequently increases the amount of T and B cell infiltration into the tumor.

### Example 4

In Vivo Efficacy and Pharmacodynamics Study with FAP-LTBR Surrogate Molecule in Combination with FolR1 TCB in a PDX Breast Cancer Model in Humanized Mice

The anti-tumor efficacy and pharmacodynamics effect on immune infiltration, HEV differentiation and cytokine secretion of P1AG7512, a fully human FAP-LTBR bispecific molecule, in monotherapy or in combination with P1AK1120, a humanFolR1-humanCD3 T cell bispecific molecule, was assessed in humanized mice bearing a human breast tumor PDX expressing human FolR1 (BC004).

BC004 was purchased from OncoTest (Freiburg, Germany). Tumor fragments were digested with Collagenase D and DNase I (Roche), counted and 1×10⁶ BC004 cells in total volume of 100 μl of a mix of RPMI and Matrigel were injected subcutaneously in the flank of anaesthetized humanized BRGS-CD47 mice with a 22G to 30G needle. Female NSG humanized BRGS-CD47 mice were generated by injection of human hematopoietic stem cells at an age of 4-5 weeks at the Jackson Laboratory. Upon confirmation of engraftment, the humanized mice were shipped to our laboratory at an age of 15-16 weeks. Upon arrival, mice were maintained for one week to get accustomed to the new environment and for observation.

Tumor growth was measured at least twice weekly using a caliper and tumor volume was calculated as follows: Tumor volume=(W²/2)×L (W: Width, L: Length). Therapy was started 28 days after tumor cell inoculation at an average tumor volume of 90-100 mm³ per group. P1AG7512 was injected at 3 mg/kg intravenously three times a week for the first two weeks and at 1 mg/kg in the following 2 weeks. P1AK1120 was injected at 0.5 mg/kg intravenously once a week. Tumor growth data expressed as mm³ and % change from baseline (e.i. tumor volume at randomization date) were plotted in GraphPad PRISM 8 and in a proprietary R-script (Waterfall plot). Time-to-event/Survival data were plotted as Kaplan-Meier plots in a proprietary R-script. In this study an “event” was defined as reaching a tumor size of 240 mm³ which was calculated to be critical tumor burden by: CTB=round (max(TV[baseline])+SD(TV[baseline])/10)*10. For the calculation of the time until the critical tumor burden is reached, a linear model of the following form was fitted for each animal: log(TV)=Day+Day2 where Day+Day2 represents the tumor growth kinetics over time. A prediction of the timepoint at which the critical tumor burden was reached was then made based on the respective model for each animal. Statistical analysis of the Kaplan-Meier plot was performed by Log-Rank test with p-values corrected for multiple testing using the Bonferroni-Holm method in R.

For cytokine analysis, serum was collected 8 days after therapy started. The Bio-Plex Pro™ Mouse Chemokine Panel 33-Plex (#12002231, BioRad, US) and the Bio-Plex Pro™ Human Chemokine Panel, 40-Plex (#171AK99MR2, BioRad, US) were used for cytokine and chemokine level measurement in the serum. The samples were prepared according to the manufacturer's instructions in a 96-well mag-plates (Bio-Plex Pro™ Flat Bottom Plates, #171025001, BioRad, US). The assay was read on the Flexmap 3D® machine (Luminex, US) following the instructions of the xPonent® software, version 4.2, provided with the machine. Data were plotted in GraphPad PRISM 8 and statistical analysis was performed by One-Way Anova tests with subsequent multiple comparison testing with Tukey correction.

The pharmacodynamic effects on immune cell infiltration and HEV differentiation were assessed by 3DIP immunofluorescence analysis. Tumor harvest was carried out 8 days after therapy started. Tumors were fixed in Cytofix (Biolegend) diluted 1:4 in PBS and later embedded in low-gelling temperature 4% Agarose (Sigma). Seventy-micrometer sections were subsequently cut in a vibratome (Leica, VT1200S). Immunofluorescence stainings were performed manually on these sections with the indicated anti-mouse antibodies: CD3 (Biolegend #300436), CD8 (Biolegend #344748), FoxP3 (Biolegend #320114), E-cadherin (R&D #AF648, conjugated in house to CF660C #92260) and Granzyme-B (Biolegend #372222). Sections were subsequently imaged on a Leica Stellaris 8 inverted confocal microscope. Cell quantification was carried out in IMARIS software (Bitplane) and plotting and statistical calculations were carried out in GraphPad Prism by One-way Anova test with subsequent multiple comparison testing with Tukey correction.

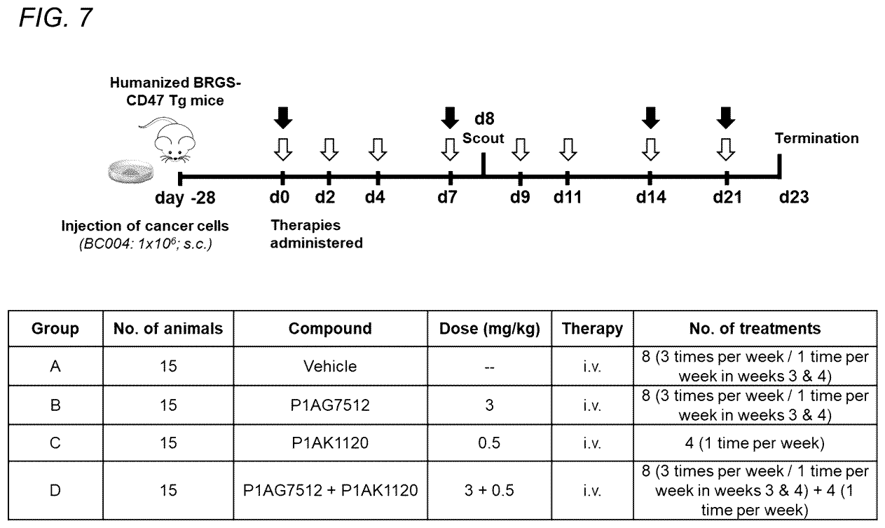

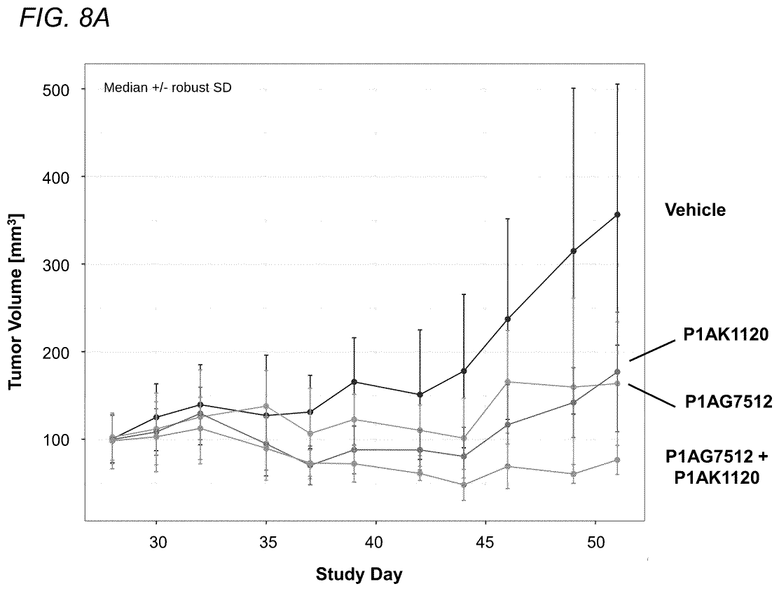

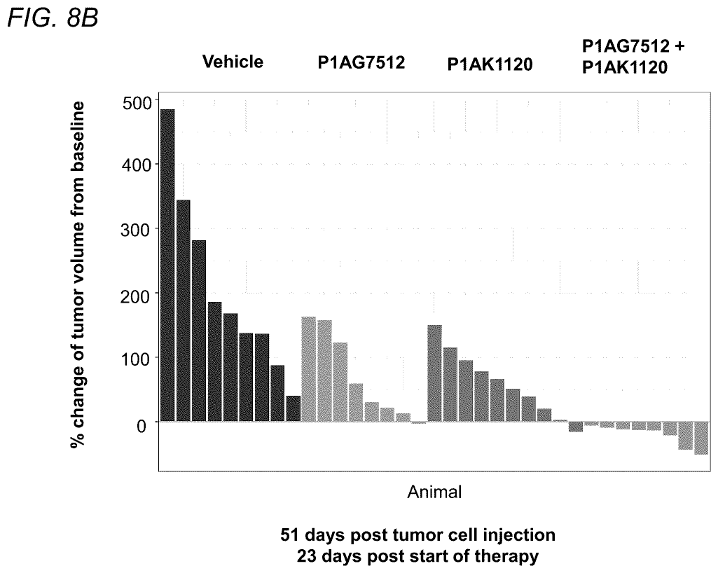

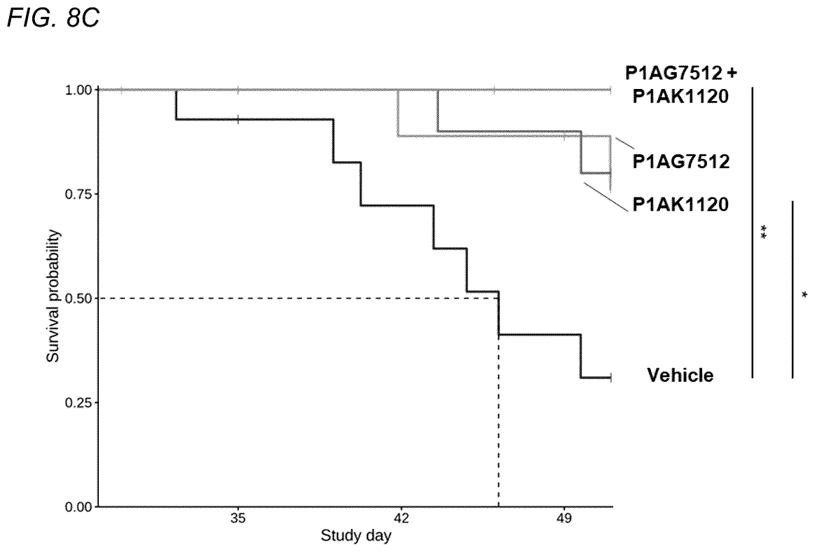

In this study (FIG. 7) we observed that combination treatment with P1AG7512 and P1AK1120 effectively controlled BC004 tumor growth (FIGS. 8A and 8B). At the end of the study, 51 days post tumor cell inoculation, 8 out of 8 mice (100%) in the combination treatment groups experienced tumor rejections, whereas only 1 out of 10 (10%) in the P1AK1120 monotherapy arm, and 1 out of 8 (12.5%) in the P1AG7512 monotherapy arm showed tumor regression (FIG. 8B). Survival/Time to event analysis showed that monotherapy with P1AK1120, and even more so, the combination of P1AG7512 and P1AK1120 improved survival/time to event as compared to vehicle control with statistical significance (p<0.005, FIG. 8C).

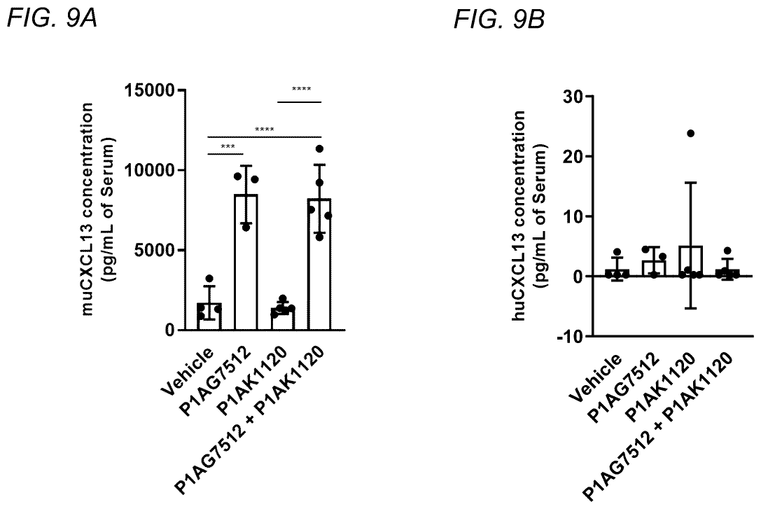

Cytokine analysis showed that treatment with P1AG7512 induced a significant increase in the levels of murine CXCL13 in the serum (FIG. 9A). Importantly, no such increase was induced by treatment with P1AK1120, suggesting that measuring CXCL13 in the serum could serve as a FAP-LTBR specific biomarker for drug activity. No increase in the levels of human CXCL13 were detected in the serum upon treatment with either therapy, suggesting that the source of CXCL13 in humanized mice is host murine cells (e.g. fibroblasts, myeloid cells) and not a human immune cell (FIG. 9B).

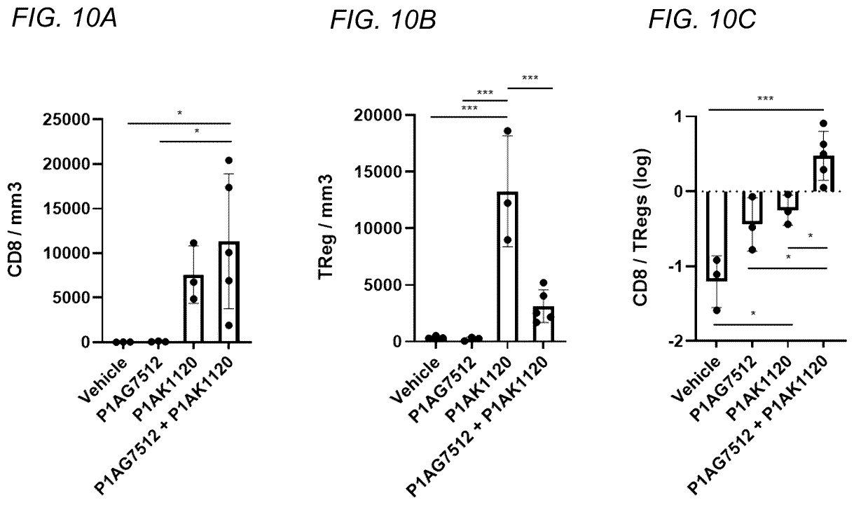

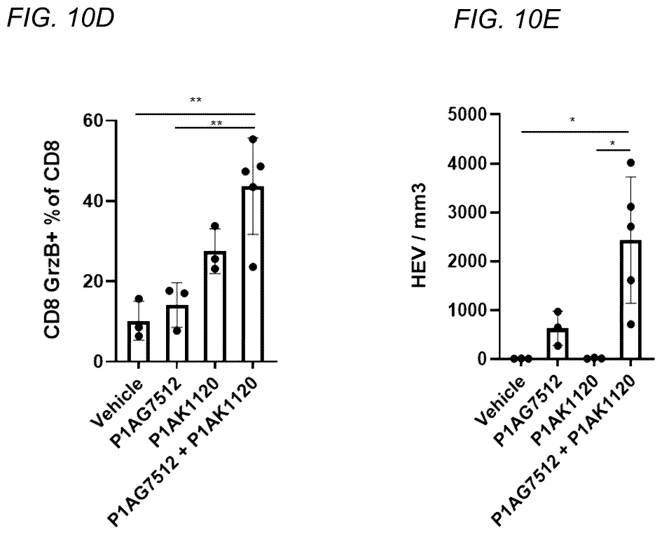

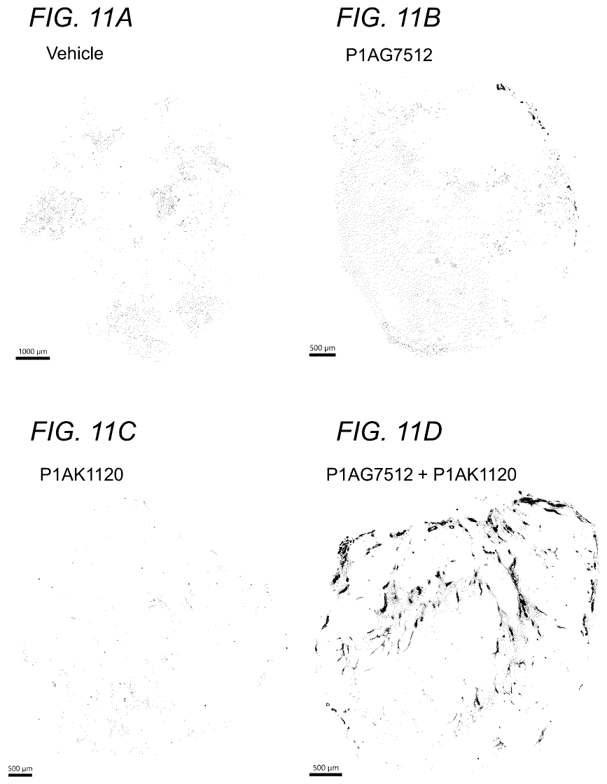

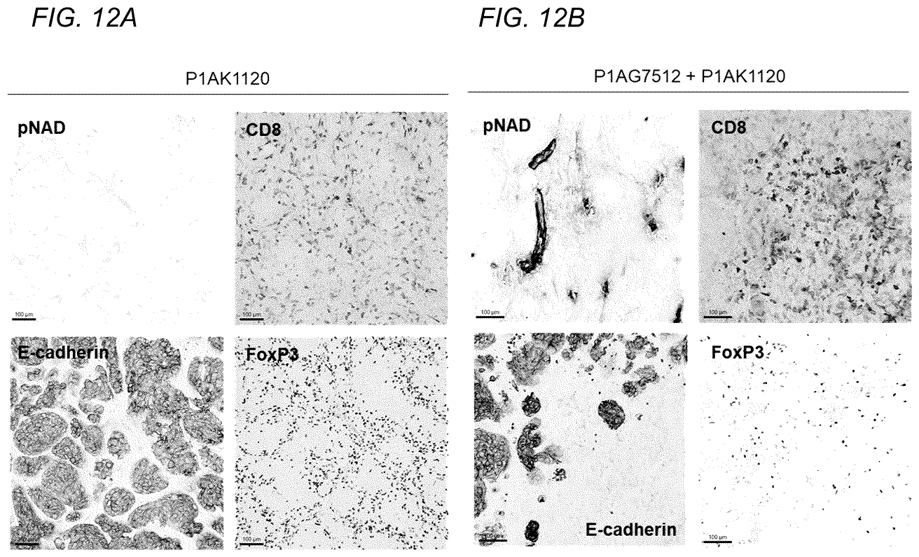

Histological analysis of tumor tissue by 3DIP immunofluorescence revealed that treatment with the combination of P1AK1120 and P1AG7512 significantly increased the infiltration of CD8 T cells (FIG. 10A) and the frequency of GrzB⁺ CD8 T cells (FIG. 10D). Treatment with P1AK1120 increased the infiltration of Treg cells, however, this increase was significantly reduced by combination treatment with P1AG7512 (FIG. 10B). Consequently, the combination therapy had the most advantageous CD8/Treg ratio (FIG. 10C). This switch to a more CD8 inflamed, Treg reduced tumor microenvironment in the combination therapy as compared to the P1AK1120 monotherapy could also be clearly appreciated by visual inspection of the tumor sections (FIG. 12A and FIG. 12B). Moreover, histological analysis revealed that blood vessel differentiation into HEVs was more pronounced in the combination therapy group compared to the P1AG7512 monotherapy group as shown by quantification (FIG. 10E) and visual inspection of the tumor sections (FIGS. 11A to 11D).

Taken together, the data described in this example show that the combination of a fully human anti FAP/anti-LTBR bispecific antibody with a T cell engager is efficacious in vivo and superior to either monotherapy in controlling growth of a breast cancer PDX model in humanized mice, suggesting that this combination can be utilized to maximize responses to T cell engagers in solid human tumors, including breast cancer. The data also show that the combination therapy modulates the tumor microenvironment in a unique way as compared to either monotherapy. Only the combination therapy induced a highly CD8 infiltrated, less Treg infiltrated and highly HEV rich tumor microenvironment that resulted in rejection of 100% of implanted tumors.

1. A T-cell engaging anti-CD3 bispecific antibody in combination with a FAP-targeted lymphotoxin beta receptor (LTBR) agonistic antibody for use in a combination therapy for the treatment of cancer in an individual.

2. A FAP-targeted LTBR agonistic antibody for use in the treatment of a cancer in an individual, wherein the treatment comprises administration of the FAP-targeted LTBR agonistic antibody in combination with a T-cell engaging anti-CD3 bispecific antibody.

3. Use of a FAP-targeted LTBR agonistic antibody in the manufacture of a medicament for the treatment of cancer in an individual, wherein the treatment comprises administration of the FAP-targeted LTBR agonistic antibody in combination with a T-cell engaging anti-CD3 bispecific antibody.

4. Use of a T-cell engaging anti-CD3 bispecific antibody in the manufacture of a medicament for the treatment of cancer in an individual, wherein the treatment comprises administration of the T-cell engaging anti-CD3 bispecific antibody in combination with a FAP-targeted LTBR agonistic antibody.

5. A method for treating cancer in an individual comprising administering to the individual an effective amount of a T-cell engaging anti-CD3 bispecific antibody and an effective amount of a FAP-targeted LTBR agonistic antibody.

6. A kit comprising a first medicament comprising T-cell engaging anti-CD3 bispecific antibody and a second medicament comprising a FAP-targeted LTBR agonistic antibody, and optionally further comprising a package insert comprising instructions for administration of the first medicament in combination with the second medicament for treating cancer in an individual.

7. The T-cell engaging anti-CD3 bispecific antibody and/or the FAP-targeted LTBR agonistic antibody for use, the use, the method or the kit of any one of the claims 1 to 6, wherein the T-cell engaging anti-CD3 bispecific antibody and the FAP-targeted LTBR agonistic antibody are administered together in a single composition or administered separately in two or more different compositions.

8. The T-cell engaging anti-CD3 bispecific antibody and/or the FAP-targeted LTBR agonistic antibody for use, the use, the method or the kit of any one of claims 1 to 7, wherein the FAP-targeted LTBR agonistic antibody acts synergistically with the T-cell engaging anti-CD3 bispecific antibody.

9. The T-cell engaging anti-CD3 bispecific antibody and/or the FAP-targeted LTBR agonistic antibody for use, the use, the method or the kit of any one of claims 1 to 8, wherein the FAP-targeted LTBR agonistic antibody comprises at least one antigen binding domain capable of specific binding to LTBR comprising a heavy chain variable region (V_{H}LTBR) comprising (i) CDR-H1 comprising the amino acid sequence of SEQ ID NO:19, (ii) CDR-H2 comprising the amino acid sequence of SEQ ID NO:20, and (iii) CDR-H3 comprising the amino acid sequence of SEQ ID NO:21, and a light chain variable region (V_{L}LTBR) comprising (iv) CDR-L1 comprising the amino acid sequence of SEQ ID NO:22, (v) CDR-L2 comprising the amino acid sequence of SEQ ID NO:23, and (vi) CDR-L3 comprising the amino acid sequence of SEQ ID NO:24.

10. The T-cell engaging anti-CD3 bispecific antibody and/or the FAP-targeted LTBR agonistic antibody for use, the use, the method or the kit of any one of claims 1 to 9, wherein the FAP-targeted LTBR agonistic antibody comprises at least one antigen binding domain capable of specific binding to LTBR comprising a heavy chain variable region (V_{H}LTBR) comprising an amino acid sequence of SEQ ID NO:25 and a light chain variable region (V_{L}LTBR) comprising an amino acid sequence of SEQ ID NO:26.

11. The T-cell engaging anti-CD3 bispecific antibody and/or the FAP-targeted LTBR agonistic antibody for use, the use, the method or the kit of any one of claims 1 to 10, wherein the FAP-targeted LTBR agonistic antibody comprises an antigen binding domain that specifically binds to FAP comprising
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13, and a light chain variable region (V_{L}FAP) comprising a CDR-L1 comprising the amino acid sequence of SEQ ID NO:14 a CDR-L2 comprising the amino acid sequence of SEQ ID NO 15 and a CDR-L3 comprising the amino acid sequence of SEQ ID NO 16.

12. The T-cell engaging anti-CD3 bispecific antibody and/or the FAP-targeted LTBR agonistic antibody for use, the use, the method or the kit of any one of claims 1 to 11, wherein the FAP-targeted LTBR agonistic antibody comprises an antigen binding domain that specifically binds to FAP comprising a heavy chain variable region (V_{H}FAP) comprising the amino acid sequence of SEQ ID NO:9 and a light chain variable region (V_{L}FAP) comprising the amino acid sequence of SEQ ID NO:10 or a heavy chain variable region (V_{H}FAP) comprising the amino acid sequence of SEQ ID NO:17 and a light chain variable region (V_{L}FAP) comprising the amino acid sequence of SEQ ID NO:18.

13. The T-cell engaging anti-CD3 bispecific antibody and/or the FAP-targeted LTBR agonistic antibody for use, the use, the method or the kit of any one of claims 1 to 12, wherein the FAP-targeted LTBR agonistic antibody comprises an IgG Fc domain, specifically an IgG1 Fc domain or an IgG4 Fc domain.

14. The T-cell engaging anti-CD3 bispecific antibody and/or the FAP-targeted LTBR agonistic antibody for use, the use, the method or the kit of claim 13, wherein the FAP-targeted LTBR agonistic antibody comprises a Fc domain with one or more amino acid substitution that reduces binding to an Fc receptor and/or effector function.

15. The T-cell engaging anti-CD3 bispecific antibody and/or the FAP-targeted LTBR agonistic antibody for use, the use, the method or the kit of any one of claims 1 to 14, wherein the FAP-targeted LTBR agonistic antibody comprises
(a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and
(b) a second antigen binding domain that specifically binds to LTBR, comprising a heavy chain variable region (V_{H} LTBR) comprising an amino acid sequence of SEQ ID NO:25 and a light chain variable region (V_{L} LTBR) comprising an amino acid sequence of SEQ ID NO:26.

16. The T-cell engaging anti-CD3 bispecific antibody and/or the FAP-targeted LTBR agonistic antibody for use, the use, the method or the kit of any one of claims 1 to 15, wherein the FAP-targeted LTBR agonistic antibody comprises
(a) a first Fab fragment that binds to FAP,
(b) a second and a third Fab fragment that bind to human LTBR, and
(c) a Fc domain of human IgG1 subclass comprising one or more amino acid substitution that reduces the binding affinity of the antigen binding molecule to an Fc receptor and/or effector function,
wherein the first Fab fragment that binds to FAP is fused at its N-terminus to the C-terminus of one of the Fc domain subunits and the second and a third Fab fragment that specifically bind to LTBR are each fused at its C-terminus to the N-terminus of one of the Fc domain subunits.

17. The T-cell engaging anti-CD3 bispecific antibody and/or the FAP-targeted LTBR agonistic antibody for use, the use, the method or the kit of any one of claims 1 to 16, wherein the FAP-targeted LTBR agonistic antibody comprises a first heavy chain comprising the amino acid sequence of SEQ ID NO:51, and a second heavy chain comprising the amino acid sequence of SEQ ID NO:53, two light chains, each comprising the amino acid sequence of SEQ ID NO:52, and one light chain comprising the amino acid sequence of SEQ ID NO:54.

18. The T-cell engaging anti-CD3 bispecific antibody and/or the FAP-targeted LTBR agonistic antibody for use, the use, the method or the kit of any one of claims 1 to 17, wherein the T-cell engaging anti-CD3 bispecific antibody specifically binds to a tumor-associated antigen.

19. The T-cell engaging anti-CD3 bispecific antibody and/or the FAP-targeted LTBR agonistic antibody for use, the use, the method or the kit of any one of claims 1 to 18, wherein the T-cell engaging anti-CD3 bispecific antibody comprises
(i) a heavy chain variable region (V_{H}CD3) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L}CD3) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
(ii) a heavy chain variable region (V_{H}CD3) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L}CD3) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40.

20. The T-cell engaging anti-CD3 bispecific antibody and/or the FAP-targeted LTBR agonistic antibody for use, the use, the method or the kit of any one of claims 1 to 19, wherein the T-cell engaging anti-CD3 bispecific antibody comprises a heavy chain variable region (V_{H}CD3) comprising the amino acid sequence of SEQ ID NO:33 and a light chain variable region (V_{L}CD3) comprising the amino acid sequence of SEQ ID NO:34 or a heavy chain variable region (V_{H}CD3) comprising the amino acid sequence of SEQ ID NO:41 and a light chain variable region (V_{L}CD3) comprising the amino acid sequence of SEQ ID NO:42.

21. The T-cell engaging anti-CD3 bispecific antibody and/or the FAP-targeted LTBR agonistic antibody for use, the use, the method or the kit of any one of claims 1 to 20, wherein the T-cell engaging anti-CD3 bispecific antibody is an anti-CEA/anti-CD3 bispecific antibody.

22. The T-cell engaging anti-CD3 bispecific antibody and/or the FAP-targeted LTBR agonistic antibody for use, the use, the method or the kit of any one of claims 1 to 21, wherein the T-cell engaging anti-CD3 bispecific antibody comprises at least one antigen binding domains capable of specific binding to CEA comprising a heavy chain variable region (V_{H}CEA) comprising CDR-H1 sequence of SEQ ID NO:43, CDR-H2 sequence of SEQ ID NO:4, and CDR-H3 sequence of SEQ ID NO:45, and a light chain variable region (V_{L}CEA) comprising CDR-L1 sequence of SEQ ID NO:46, CDR-L2 sequence of SEQ ID NO:47, and CDR-L3 sequence of SEQ ID NO:48.

23. The T-cell engaging anti-CD3 bispecific antibody and/or the FAP-targeted LTBR agonistic antibody for use, the use, the method or the kit of any one of claims 1 to 22, wherein the T-cell engaging anti-CD3 bispecific antibody comprises at least one antigen binding domains capable of specific binding to CEA comprising a heavy chain variable region (V_{H}CEA) comprising the amino acid sequence of SEQ ID NO:49 and a light chain variable region (V_{L}CEA) comprising the amino acid sequence of SEQ ID NO:50.

24. The T-cell engaging anti-CD3 bispecific antibody and/or the FAP-targeted LTBR agonistic antibody for use, the use, the method or the kit of any one of claims 1 to 23, wherein the T-cell engaging anti-CD3 bispecific antibody comprises two antigen binding domains that bind to CEA.

25. The T-cell engaging anti-CD3 bispecific antibody and/or the FAP-targeted LTBR agonistic antibody for use, the use, the method or the kit of any one of claims 1 to 24, wherein the T-cell engaging anti-CD3 bispecific antibody comprises an Fc domain comprising one or more amino acid substitutions that reduce binding to an Fc receptor and/or effector function.

26. The T-cell engaging anti-CD3 bispecific antibody and/or the FAP-targeted LTBR agonistic antibody for use, the use, the method or the kit of any one of claims 1 to 25, wherein the T-cell engaging anti-CD3 bispecific antibody comprises a first heavy chain comprising the amino acid sequence of SEQ ID NO:61, and a second heavy chain comprising the amino acid sequence of SEQ ID NO:62, two light chains, each comprising the amino acid sequence of SEQ ID NO:59, and one light chain comprising the amino acid sequence of SEQ ID NO:60.

27. The T-cell engaging anti-CD3 bispecific antibody and/or the FAP-targeted LTBR agonistic antibody for use, the use, the method or the kit of any one of claims 1 to 18, wherein the T-cell engaging anti-CD3 bispecific antibody is an anti-FolR1/anti-CD3 bispecific antibody.

28. The T-cell engaging anti-CD3 bispecific antibody and/or the FAP-targeted LTBR agonistic antibody for use, the use, the method or the kit of any one of claims 1 to 18 or 27, wherein the T-cell engaging anti-CD3 bispecific antibody comprises at least one antigen binding domains capable of specific binding to FolR1 comprising a heavy chain variable region (V_{H}FolR1) comprising the CDR-H1 sequence of SEQ ID NO:111, the CDR-H2 sequence of SEQ ID NO:112, and the CDR-H3 sequence of SEQ ID NO:113, and a light chain variable region (V_{L}FolR1) comprising the CDR-L1 sequence of SEQ ID NO:114, the CDR-L2 sequence of SEQ ID NO:115, and the CDR-L3 sequence of SEQ ID NO:116.

29. The T-cell engaging anti-CD3 bispecific antibody and/or the FAP-targeted LTBR agonistic antibody for use, the use, the method or the kit of any one of claims 1 to 18 or 27 or 28, wherein the T-cell engaging anti-CD3 bispecific antibody comprises at least one antigen binding domains capable of specific binding to FolR1 comprising a heavy chain variable region (V_{H}FolR1) comprising the amino acid sequence of SEQ ID NO:118 and a light chain variable region (V_{L}FolR1) comprising the amino acid sequence of SEQ ID NO:119.

30. The T-cell engaging anti-CD3 bispecific antibody and/or the FAP-targeted LTBR agonistic antibody for use, the use, the method or the kit of any one of claims 1 to 18 or 27 to 29, wherein the T-cell engaging anti-CD3 bispecific antibody comprises two antigen binding domains that bind to FolR1.

31. The T-cell engaging anti-CD3 bispecific antibody and/or the FAP-targeted LTBR agonistic antibody for use, the use, the method or the kit of any one of claims 1 to 18 or 27 to 30, wherein the T-cell engaging anti-CD3 bispecific antibody comprises a first antigen binding domain comprising a heavy chain variable region (V_{H}CD3), a second antigen binding domain comprising a heavy chain variable region (V_{H}FolR1), a third antigen binding domain comprising a heavy chain variable region (V_{H}FolR1) and three times a common light chain variable region.

32. The T-cell engaging anti-CD3 bispecific antibody and/or the FAP-targeted LTBR agonistic antibody for use, the use, the method or the kit of any one of claims 1 to 18 or 27 to 31, wherein the T-cell engaging anti-CD3 bispecific antibody comprises a first heavy chain comprising the amino acid sequence of SEQ ID NO:120, a second heavy chain comprising the amino acid sequence of SEQ ID NO:121 and three times a common light chain of SEQ ID NO: 122.

33. The T-cell engaging anti-CD3 bispecific antibody and/or the FAP-targeted LTBR agonistic antibody for use, the use, the method or the kit of any one of claims 1 to 32, wherein the T-cell engaging anti-CD3 bispecific antibody and the FAP-targeted LTBR agonistic antibody are administered intravenously or subcutaneously.

34. The T-cell engaging anti-CD3 bispecific antibody and/or the FAP-targeted LTBR agonistic antibody for use, the use, the method or the kit of any one of claims 1 to 33, wherein the FAP-targeted LTBR agonistic antibody is administered subcutaneously.

35. The T-cell engaging anti-CD3 bispecific antibody and/or the FAP-targeted LTBR agonistic antibody for use, the use, the method or the kit of any one of claims 1 to 34, wherein the T-cell engaging anti-CD3 bispecific antibody is administered concurrently with, prior to, or subsequently to the FAP-targeted LTBR agonistic antibody.
